(19) United States
(12) Patent Application Publication
(10) Pub. No.: US 2024/0254431 A1
(43) Pub. Date: Aug. 1, 2024

(54) SYSTEMS AND METHODS FOR HIGH-THROUGHPUT CELL LINE DEVELOPMENT

(71) Applicant: ONECYTE BIOTECHNOLOGIES, INC., Cambridge, MA (US)

(72) Inventors: Konstantinos Tsioris, Swampscott, MA (US); Ayca Yalcin Ozkumur, Newton Highlands, MA (US)

(73) Assignee: ONECYTE BIOTECHNOLOGIES, INC., Cambridge, MA (US)

(21) Appl. No.: 18/034,488

(22) PCT Filed: Oct. 29, 2021
(86) PCT No.: PCT/US2021/057453

§ 371 Date: Apr. 28, 2023

Related U.S. Application Data
(60) Provisional application No. 63/192,305, filed on May 24, 2021.
(60) Provisional application No. 63/107,967, filed on Oct. 30, 2020.

(51) Int. Cl. C12M 1/00; C12M 1/32; C12M 1/36; C12M 3/06; G06T 7/00

(52) U.S. Cl. CPC C12M 47/04; C12M 23/12; C12M 23/16; C12M 41/48; G06T 7/0012; G06T 2207/10056; G06T 2207/20081

(57) ABSTRACT

Provided herein are methods and systems for high-throughput cell line development.

### CROSS-REFERENCE

This application claims the benefit of U.S. Provisional Application No. 63/107,967 filed Oct. 30, 2020, U.S. Provisional Patent Application No. 63/192,305, filed May 24, 2021, each of which is entirely incorporated herein by reference.

### STATEMENT REGARDING FEDERALLY SPONSORED RESEARCH OR DEVELOPMENT

This invention was made with government support under Grant No. 2032448 awarded by the National Science Foundation. The government has certain rights in the invention.

### BACKGROUND

In cellular biology and related fields, single-cell analysis is a useful tool to study genomics, transcriptomics, proteomics, metabolomics and cell-cell interactions at the single cell level. Since both eukaryotic and prokaryotic cell populations are heterogeneity, analyzing a single cell among a population of cells can allow the precise selection of desirable single cells for various applications from monoclonal antibody production to cell line development.

### SUMMARY

Provided herein are systems and methods for high-throughput cell line development, providing for rapid identification and characterization of compositions produced by cells. Additionally, the systems and methods disclosed herein provide for rapid identification and characterization of cells as well.

One aspect of the present disclosure provides a method for selecting a target cell, comprising: a) placing a plurality of cells into a plurality of chambers, wherein each individual chamber of a subset of the plurality of chambers contains one or no more than 2, 3, 5, 10, 15 or 20 individual cells of the plurality of cells; b) exposing at least the subset of the plurality of chambers from a) to a condition, wherein the condition is exposing the individual chamber with one or more regents, or treating the individual chamber with a plurality of secondary cells, or applying a membrane to the individual chamber to form an individual membrane-modified chamber, or contacting the individual chamber with a capture substrate, or contacting the individual chamber with a secondary cell-immobilized capture substrate, or a combination thereof; c) detecting a signal or a change thereof from a particular chamber of the subset of the plurality of chambers during or after the exposing in b), wherein the signal or the change thereof is indicative of (i) the presence of a target cell in the particular chamber, or (ii) the presence of a product produced by the target cell in the particular chamber; and d) selecting the target cell in the particular chamber from the plurality of cells at least based on a pre-determined value of the signal or the change thereof in c).

In some embodiments, the method further comprises: e) transferring the target cell selected in d) to a cultivation vessel, and expanding the target cell into a colony or colonies in the cultivation vessel. In some embodiments, the selecting in d) comprises predicting an expected outcome of the colony or colonies in e) based on the signal or the change thereof in c). In some embodiments, the method is further characterized in that: A) the plurality of cells in a) are from about 100 to about 1,000,000 heterogenous cells; and/or B) a solution volume of the individual chamber is from 100 picoliter to 900 nanoliter; and/or C) completing step a) is done in no more than 10, 9, 8, 7, 6, 5, 4, 3, 2, or 1 minute(s); and/or D) completing steps a) to d) is done in no more than 48, 36, 24, 12, 10, 9, 8, 7, 6, 5, 4, 3, 2, or 1 hour, or 30, 20, 10, 5 minutes and/or E) the detecting in c) is cell morphology imaging, near-infrared imaging, fluorescence imaging, luminescence imaging, UV-vis imaging, brightfield imaging, hyperspectral imaging, surface plasmon resonance (SPR) imaging, imaging with optical fibers, label-free imaging, mass spectrometry, or a combination thereof, and/or F) the selecting in d) comprises analyzing (i) the signal or the change thereof, and/or (ii) an additional signal or a change thereof obtained from the colony or colonies in e), wherein the analyzing in F) is machine learning-based, or artificial intelligence (AI)-based, or deep learning-based, or neural networks-based, or a combination thereof, and/or G) the expected outcome of an outgrowth population of the colony or colonies correlates with an observed outcome of the outgrowth population of the colony or colonies in e). In some embodiments, the colony or colonies in e) displays higher monoclonality assurance when compared with a comparative colony or colonies obtained by (i) limiting dilution selection, (ii) fluorescence-activated cell sorting (FACS), (iii) isolating individual cells with cloning cylinders, or (iv) flow cytometry. In some embodiments, the colony or colonies in e) displays higher viability when compared with a comparative colony or colonies obtained by (i) limiting dilution selection, (ii) fluorescence-activated cell sorting (FACS), (iii) isolating individual cells with cloning cylinders, or (iv) flow cytometry. In some embodiments, the analyzing in F) comprises further analyzing intracellular staining for the product, and/or surface markers, and/or the cell morphology imaging against an optimized machine learning model built on correlating cell intracellular staining features, and/or surface markers, and/or cell morphological features of selected single cells with the corresponding product attribute parameters of the outgrowth populations derived from the selected single cells. In some embodiments, the completing steps a) to d) in D) is from 48 to 36 hours, from 36 to 24 hours, from 24 to 12 hours, from 12 to 10 hours, from 10 to 9 hours, from 9 to 8 hours, from 8 to 7 hours, from 7 to 6 hours, from 6 to 5 hours, from 5 to 4 hours, from 4 to 3 hours, from 3 to 2 hours, from 2 to 1 hour(s), from 60 to 30 minutes, and from 30 to 1 minute(s). In some embodiments, the completing steps a) to d) in D) is faster than when a comparative colony or colonies is obtained by (i) limiting dilution selection, or (ii) fluorescence-activated cell sorting (FACS), or (iii) isolating individual cells with cloning cylinders, or (iv) flow cytometry. In some embodiments, completing steps b) to d) is done from 30 to 5 minutes, from 20 to 5 minutes, from 15 to 5 minutes, from 10 to 5 minutes. In some embodiments, completing step d) is done from 10 to 9 minutes, from 9 to 8 minutes, from 8 to 7 minutes, from 7 to 6 minutes, from 6 to 5 minutes, from 5 to 4 minutes, from 4 to 3 minutes, from 3 to 2 minutes, from 2 to 1 minute(s), from 60 to 30 seconds, and from 30 to 1 second(s). In some embodiments, steps b) and c) are performed while the plurality of cells receive reduced perturbations when compared with corresponding perturbations received by a comparative plurality of cells in a cell line development process of (i) limiting dilution selection, (ii) fluorescence-activated cell sorting (FACS), (iii) isolating individual cells with cloning cylinders, or (iv) flow cytometry. In some embodiments, the perturbations are chemical, biological, or mechanical perturbations with regard to the plurality cells or the solution/environment of the plurality of cells.

In some embodiments, the target cell is not removed from the particular chamber before step d) is completed. In some embodiments, the outcome comprise titer, cell growth metric, viable cell density, characteristics, expression of surface glycoproteins, glycosylation, phosphorylation, deamidation, methylation, acetylation aggregation, monoclonality, expression of cell markers, biological activities, or impurities. In some embodiments, the analyzing in F) improves the correlation of the expected outcome of the outgrowth population of the colony or colonies with the observed outcome of an outgrowth population of the colony or colonies in e). In some embodiments, the product is an antibody, a monoclonal antibody, a biosimilar, a virus, a protein, a nucleotide, a bispecific, an antibody-drug conjugate, an exosome, a biomarker, or a metabolite.

Another aspect of the present disclosure relates to a method for facilitating colony or colonies selection of a cell line, from among a plurality of candidate single cells, comprising: a) generating, by an imaging unit, a first plurality of images of each of the plurality of candidate single cells individually, wherein each of the plurality of candidate single cells resides in an individual chamber of a plurality of chambers; b) detecting, by one or more processors analyzing the first plurality of images for each of the plurality of candidate single cells, one or more cell features of each of the plurality of candidate single cells depicted in the first plurality of images; and c) based on the one or more cell features, determining, by the one or more processors and according to a finalized single cell-to-colony machine learning model, one or more predicted attributes for a colony expanded from each of the plurality of candidate single cells; d) ranking the plurality of candidate single cells according to the one or more predicted attributes for each of the plurality of candidate single cells, wherein the finalized single cell-to-colony model predicts attributes of a hypothetical colony based on at least the one or more cell features of a single cell.

In some embodiments, the one or more cell features are morphological cell features of shape, size, color, pattern, texture, nucleus size, or organelles, or intracellular staining for a product produced by the single cell, or one or more surface markers, or a combination thereof. In some embodiments, the one or more predicted attributes are titer, cell growth metric, viable cell density, characteristics, expression of surface glycoproteins, glycosylation, phosphorylation, deamidation, methylation, acetylation aggregation, monoclonality, expression of cell markers, biological activities, or impurities. In some embodiments, the finalized single cell-to-colony model is optimized by using a training data set comprising (i) the one or more morphological cell features from a second plurality of images for a plurality of training single cells, and (ii) measured quality attributes of each colony expanded from each of the plurality of training single cells. In some embodiments, the finalized single cell-to-colony model is further optimized by (a) using a validation data set comprising (i) the one or more morphological cell features from a third plurality of images for a plurality of validation single cells, and (ii) measured quality attributes of each colony expanded from each of the plurality of validation single cells, and (b) comparing one or more predicted attributes of each of the plurality of validation single cells with the measured attributes of each of the colony expanded from each of the plurality of validation single cells.

Described herein are certain aspects of a method for high-throughput cell line development, comprising: providing a plurality of target cells, an array of nano-wells, one or more reagents and instructions; loading the plurality of target cells into the array of nano-wells such that an individual well of the array of nano-wells contains an individual target cell; exposing the plurality of target cells to one or more reagents; obtaining quantitative measurements of individual target cells and quantitative measurements of individual articles associated with the individual target cells; selecting a target cell from the individual target cells to be recovered based on predetermined values of the quantitative measurements; wherein a time to reach a decision for selecting the target cell for recovery does not exceed 3 hours from the initialization of the method; and wherein the method yields clones with a mean productivity of at least 5 grams per liter. In certain aspects, the method yields clones with a mean productivity of 2 grams. In certain aspects, the method yields clones with a mean productivity of 4 grams. In certain aspects, a capture substrate is provided, further wherein one or more binding molecules for the article is immobilized to the capture substrate. In certain aspects, the capture substrate is placed in proximity of the array of nano-wells before, during or after exposure of the one or more reagents to the target cells. In certain aspects, measurements of the articles are obtained on a surface of the capture substrate. In some embodiments, the measurements of the articles obtained on the surface of the capture substrate comprise optical analytics. In some embodiments, the article is a biomolecule. In some embodiments, the biomolecule is synthetically derived. In some embodiments, the biomolecule is naturally derived. In some embodiments, the biomolecule is a biomolecule comprising an Fc domain. In some embodiments, the biomolecule comprising an Fc domain is an antibody. In some embodiments, the article is secreted by the target cell. In some embodiments, the article is a bioparticle. In some embodiments, the article is presented on the surface of the target cell. In some embodiments, the article is internal to the target cell. In some embodiments, the biomolecule is encoded by a heterologous gene. In some embodiments, the target cell is a T cell, an antibody secreting cell, a B cell, a plasma cell, a hybridoma, an immune cell, or an engineered cell. In some embodiments, wherein the engineered cell is a CHO cell, or HEK cell. In some embodiments, the biomolecule binds to one or more antigens that are markers for infection. In some embodiments, the infection is a viral infection, a parasitic infection, a bacterial infection, or a bioweapon-based infection. In some embodiments, the viral infection is COVID-19. In some embodiments, the infection is known to cause epidemic or pandemic levels of infection. In some embodiments, the one or more reagents comprise one or more secondary cell, reporter cell, perturbing cell, one or more cellular factors, media, antigen, secondary binding molecule, labeling molecule, or a combination thereof. In some embodiments, the one or more cellular factors are capable of modifying a cell in terms of parameters comprising growth, gene and protein expression, up-regulation, down-regulation, function, specificity, developmental timing, niche occupation, differentiation, de-differentiation, methylation, productivity, stability, glycosylation, aggregation, recombinant modification, genetic modification, transcriptional modification, modifications and interactions with proteins, methylation, ubiquitination, phosphorylation, or other perturbations. In some embodiments, the number of target cells per array of nano-wells is less than or equal to 16,000. In some embodiments, the number of target cells per array of nano-wells is less than or equal to 27,000. In some embodiments, the number of target cells per array of nano-wells is less than or equal to 300,000. In some embodiments, the number of target cells per array of nano-wells is less than or equal to 5,000. In some embodiments, the volume of the target cells in a sample does not exceed 0.2 milliliters. In some embodiments, the number of the target cells in a sample does not exceed 200,000 per milliliter. In some embodiments, the number of the target cells in a sample does not exceed 20,000 per milliliter. In some embodiments, the number of the target cells in a sample does not exceed 10,000 per milliliter. In some embodiments, the number of the target cells in a sample does not exceed 2,000 per milliliter. In some embodiments, the single-cell loading efficiency of cells is 33%. In some embodiments, the single-cell loading efficiency of cells is 20%. In some embodiments, the time for loading the individual target cells into the array of nano-wells and the secretion assay of the individual cells does not exceed 11 minutes. In some embodiments, the time for loading the individual target cells into the array of nano-wells and secretion assay of the individual target cells does not exceed 6 minutes. In some embodiments, the time for capturing biomolecules on the capture substrate after sealing the array of nano-wells does not exceed 29 minutes. In some embodiments, the time for capturing biomolecules on the capture substrate surface after sealing the array of nano-wells does not exceed 11 minutes. In some embodiments, the time for capturing biomolecules on the capture substrate after sealing the array of nano-wells does not exceed 4 minutes. In some embodiments, the target cell does not contact detection reagents. In some embodiments, the time to reach a decision for selecting the target cell does not exceed 2 hours from initialization of the method. In some embodiments, the time to reach a decision for selecting the target cell does not exceed 4 hours from initialization of the method. In some embodiments, the time to reach a decision for selecting the target cell does not exceed 5 hours from initialization of the method. In some embodiments, the time to reach a decision for selecting the target cell does not exceed 1 hour from initialization of the method. In some embodiments, the time to reach the decision for selecting the target cell does not exceed 5 doubling times. In some embodiments, the time to reach the decision for selecting the target cell does not exceed 1 doubling time. In some embodiments, the method yields clones with a mean productivity within a range of a 5 to 12 grams per liter. In some embodiments, the method yields clones with a mean productivity within a range of 1 to 5 grams per liter. In some embodiments, the method yields clones with a mean productivity within a range of 0.1 to 1 gram per liter. In some embodiments, a collection of proof images is acquired at each step during the method. In some embodiments, the capture substrate is comprised of a hard material. In some embodiments, the capture substrate is comprised of a soft material. In some embodiments, the array of nano-wells is comprised of a hard material. In some embodiments, the array of nano-wells is comprised of a soft material. In some embodiments, the hard material comprises a transparent plastic or a transparent glass material. In some embodiments, the substrate comprises a reflective material. In some embodiments, the soft material comprises a transparent elastomeric material. In some embodiments, the article is captured on the capture substrate. In some embodiments, the article is captured on one or a plurality of beads inside of the well. In some embodiments, the article is captured on an interior surface of the well. In some embodiments, the article is captured within a matrix contained within the well. In some embodiments, the measurements of individual target cells comprise characterizations of cellular objects, through segmentation or without segmentation, such as morphology, size, texture of nucleolus, endoplasmic reticulum, nucleoli, cytoplasmic RNA, actin, cytoskeleton, golgi, plasma membrane, mitochondria and other organelles or cell components or a combination thereof. In some embodiments, data from the measurements of individual target cells is used to create a training data set to predict cellular function. In some embodiments, the transgene is selected from the group consisting of amino acid (aa) pattern recognition receptor, killer activated receptor, killer inhibitor receptor, complement receptor, Fc receptor, major histocompatibility complex (MHC) molecule, human leukocyte antigen complex (HLA), cluster of differentiation (CD) markers, B cell receptor, T cell receptor, and a chimeric antigen receptor. In some embodiments, the direct measurements comprise bright field microscopy. In some embodiments, the direct measurements comprise fluorescence microscopy.

Described herein, in some embodiments, is a method for isolated co-culture utilizing a secondary cell suspension, comprising: providing a plurality of target cells, an array of nano-wells, one or more reagents and instructions; loading individual target cells of the plurality of target cells into the array of nano-wells; applying a membrane to the array of nano-wells to form a membrane-modified array of nano-wells; providing a suspension of a plurality of secondary cells, one or more reagents, or a combination thereof, near or in contact with the membrane-modified array of nano-wells; obtaining measurements of individual target cells and measurements of individual articles associated with the individual target cells; selecting a target cell from the individual target cells to be recovered based on predetermined values of the measurements; and wherein a time to reach a decision for selecting the target cell for recovery does not exceed 3 hours from the initialization of the method. In some embodiments, the plurality of secondary cells reside in a chamber that is fluidically connected to a flow cell containing the membrane-modified array of nano-wells. In some embodiments, the flow rate of the secondary cell suspension, the one or more reagents, or a combination thereof is equal to or greater than about 0 milliliters per minute.

Described herein, in certain circumstances, is a method for isolated co-culture utilizing a secondary cell immobilized-capture substrate, comprising: providing a plurality of target cells, an array of nano-wells, one or more reagents, instructions; and a plurality of secondary cells immobilized to a capture substrate; loading individual target cells of the plurality of target cells into the array of nano-wells; applying a membrane to the array of nano-wells to form a membrane-modified array of nano-wells; simultaneously contacting the membrane-modified array of nano-wells and the secondary cell-immobilized capture substrate with one or more reagents; obtaining measurements of individual target cells and measurements of individual articles associated with the individual target cells; selecting a target cell from the individual target cells to be recovered based on predetermined values of the measurements; wherein a time to reach a decision for selecting the target cell for recovery does not exceed 3 hours from the initialization of the method; and wherein the method yields clones with a mean productivity of 5 grams per liter.

Described herein, in certain circumstances, is a system for high-throughput cell line development, comprising: an array of nano-wells comprising individual nano-wells, wherein the individual nano-wells contain zero or more target cells; an apparatus for reversibly sealing a capture substrate with the array of nano-wells; a reagent module configured for supplying one or more reagents to the array of nano-wells; a detection module configured for performing measurements of biomolecules secreted by the target cell onto the capture substrate at discrete positions indexed to the individual wells; a cell recovery apparatus configured for recovery of the individual cells, wherein values extracted from the measurements of biomolecules and cells are compared to predetermined criteria and used for the selection of the individual cells to be recovered; wherein the system is configured to reach a decision for selecting the target cell for recovery within 3 hours from initialization; and wherein the system is configured to yield clones with a mean productivity of 5 grams per liter. In certain aspects, the system comprises an apparatus configured for sealing a capture substrate to the array of nano-wells, whereupon sealing a substantially aligned and substantially fluid tight seal between the one or more capture substrates and the one or more array of nano-wells is made. In certain aspects, the direct measurements comprise bright field microscopy measurements. In certain aspects, the direct measurements comprise microscopy measurements utilizing a laser source and a photomultiplier tube for detection. In certain aspects, the system comprises a controller configured for actuating the system and analyzing data. In certain aspects, a well of the array of nano-wells has a diameter of 5 to 150 microns. In certain aspects, the well has a volume of picoliters to 15 nanoliters. In some embodiments, the well has a volume of 250 picoliters. In some embodiments, the well comprises shapes of circle, oval, square, triangle, diamond, or rectangle or combination thereof. In certain aspects, a well of the array of nano-wells has a depth of 25 microns. In some embodiments, a well of the array of nano-wells has a depth of 100 microns. In some embodiments, a well of the array of nano-wells has a depth of 250 microns. In some embodiments, a well of the array of nano-wells has a diameter to depth ratio of 1/10 to 4. In certain aspects, the number of wells per array is about 1 million to about 10 million. In some embodiments, the number of wells per array is about 100,000 to about 1 million. In certain aspects, the number of wells per array is about 10,000 to about 100,000. In some embodiments, the number of cells per a well of the array of nano-wells from zero to about 10. In some embodiments, a plate comprises a plurality of the array of nano-wells. In some embodiments, the plate comprises a plurality of recesses. In some embodiments, a recess of the plurality of recesses comprises an array of nano-wells. In some embodiments, the capture substrate comprises a sensing surface. In some embodiments, the array of nano-wells comprises the sensing surface. In some embodiments, the sensing surface comprises a layered semiconductor. In some embodiments, the sensing surface is configured for reflection mode imaging for real-time endpoint detection of binding on the sensing surface. In some embodiments, the sensing surface is configured for surface plasmon resonance detection of the articles. In some embodiments, the sensing surface is configured for interferometric detection of the articles. In some embodiments, the sensing surface is configured for whispering gallery mode detection of the articles.

Described herein are certain aspects for a mechanism sealing a capture substrate to an array of nano-wells in a sterile fashion, comprising: a top piece configured to immobilize capture substrate; a base configured to immobilize an array of nano-wells; wherein the base comprises one or more alignment rods to align the top piece to the base such that the capture substrate and the array of nano-wells are fixed in coplanar and rotationally aligned orientation; and wherein the distance between the capture substrate and the array of nano-wells can be controllably varied along an axis perpendicular to the coplanar planes of the capture substrate and the array of nano-wells, thus placing the capture substrate and the array of nano-wells in alignment and forming a sterile, fluid tight seal. In certain aspects, the distance is minimized to form a seal between the capture substrate and the array of nano-wells that is substantially aligned and substantially fluid tight. In certain aspects, the capture substrate is aligned with the array of nano-well in a coplanar orientation and in proximity simultaneously with a plurality of capture substrates and a plurality of array of nano-wells. In certain aspects, one or more of the array of nano-wells are contained within a plate. In certain aspects, the plate comprises one or more recesses, wherein each recess contains one or more of the arrays of nano-wells. In certain aspects, the plate comprises one or more recesses, wherein an array of nano-wells can be placed and removed from a recess of the one or more recesses. In some embodiments, a specific force is applied equally across a region of the capture substrate, the array of nano-wells, or a combination of both wherein a predetermined pressure applied across the region is substantially uniform. In some embodiments, the recess comprises one or more channels configured to accept fluid displaced between the capture substrate and the array of nano-wells. In some embodiments, the recess comprises one or more ridges to contain and align the capture substrate relative to the array of nano-wells. In some embodiments, the recess further comprises an alignment recess configured to align the capture substrate relative to the array of nano-wells. In some embodiments, the recess contains channels configured to form a pedestal and wherein the capture substrate contains a capture substrate-recess configured to accept the pedestal, allowing for alignment between the capture substrate and the pedestal. In some embodiments, the plate is in fluidic connection with one or more reservoirs wherein the one or more reservoirs contain the one or more reagents.

Described herein are certain aspects, for a method for cell line development utilizing a terminal assay for cell selection, comprising: providing a plurality of target cells, an array of nano-wells, one or more reagents and instructions; loading individual target cells of the plurality of target cells into individual nano-wells of the array of nano-wells and exposing the individual target cells to one or more reagents; contacting a capture substrate to the array of nano-wells, thereby sealing the individual target cells into the individual nano-wells; growing a colony of cells from the individual target cell, such that one or more colony cells of the colony of cells are transferred to the capture substrate onto positions registered to corresponding individual wells from which the one or more colony cells originated; separating the capture substrate from the array of nano-wells and performing an assay that may result in the death of the colony cells that transferred to the indexed positions; and selecting colony cells for recovery from the corresponding individual wells based on predetermined values of the measurements. In certain aspects, a capture substrate is sealed onto the array of nano-wells, wherein each well is sealed by the capture substrate, wherein some cells of the single-well colony are attached to the capture substrate at locations on the capture substrate in which the locations are registered to the well position in the array of nano-wells. In certain aspects, the capture substrate is separated from the array of nano-wells and measurements are performed on the some cells that are attached to the capture substrate at the locations. In certain aspects, the measurements are performed on the individual cells in the individual wells, prior to colony growth. In certain aspects, the measurements are performed on the single-well colony of target cells. In certain aspects, the measurements comprise image cytometry or a secretion assay. In some embodiments, the measurements are used to determine identity of the individual cells. In some embodiments, living cells within the single-well colony of target cells or clones are recovered based from the array of nano-wells or the capture substrate or a combination thereof.

As used herein, the term “colony” refers to colonies of single cells, as the term is commonly understood in the art of cell culture, monolayers of cells growing in a culture vessel or cultivation vessel, and other such cell layers or aggregates resulting from growth of single cells in culture.

Described herein are certain aspects for a method for high-throughput identification of a B cell or antibody secreting cells (ASCs), comprising: obtaining a plurality of B cells or ASCs from a subject; loading the individual B cells or ASCs into individual wells of an array of nano-wells; detecting a secreted product of the individual B-cell or ASCs; selecting the individual B-cell or ASCs; wherein a time to reach a decision for selecting the target cell does not exceed 5.5 hours from the initialization of the method. In certain aspects, the subject has been immunized naturally through infection with pathogenic agent. In certain aspects, the pathogenic agent is a virus selected from the group consisting of: SARS-CoV-2, Herpes simplex virus (HSV), varicella zoster virus, cytomegalovirus (CMV), Epstein-Barr virus (EBV), Eastern equine encephalitis (EEE), western equine encephalitis (WEE), rubella virus, poliovirus, coxsackievirus, an enterovirus, St. Louis encephalitis (SLE), Japanese encephalitis, rubeola (measles) virus, mumps virus, California encephalitis, LaCrosse virus, human immunodeficiency virus (HIV), rabies virus, WNV, dengue, AAV and Influenza A virus. In some embodiments, the subject has been immunized with a target antigen. In some embodiments, the subject comprises a human. In some embodiments, the subject comprises a non-human. In some embodiments, the antigen comprises a viral antigen, self-antigen or tumor antigen. In some embodiments, the property comprise an article produced by an individual B-cell or ASC. In some embodiments, the article comprises an antibody. In some embodiments, the article comprises a secreted molecule. In some embodiments, the secreted molecule comprises a cytokine. In some embodiments, the property comprises an interaction between the individual B cell and a second cell or second biomolecule.

Described herein are certain aspect for a method of cell line development utilizing identification of glycosylation patterns on a biomolecule, comprising: providing a plurality of target cells, an array of nano-wells, one or more reagents and instructions; loading the plurality of target cells into an array of nano-wells such that an individual well of the array of nano-wells contains an individual target cell; exposing the plurality of target cells to one or more reagents, wherein each individual target cell can produce a biomolecule; capturing the biomolecule produced by each individual target cell on a capture substrate; wherein the capture substrate is configured to keep the biomolecule produced by each individual target cell in the array of nano-wells spatially distinct and registered to the individual nano-well of origin; wherein the capture substrate comprises a glycan binding reagent; comparing the captured biomolecule produced by each individual target cell in the array of nano-wells on the capture substrate to a reference; ascertaining a glycosylation profile for each individual biomolecule; and selecting a target cell from the plurality of target cells to be recovered based on the glycosylation profile of the biomolecule produced by the individual target cell. In certain aspects, a time to reach a decision for selecting the individual target cell for recovery does not exceed 3 hours from the initialization of the method. In certain aspects, the method yields clones with differentiated glycan profiles. In certain aspects, capture substrates are used to create sequential prints. In certain aspects, the glycan binding reagent is a lectin, an antibody, or an antibody mimetic. In certain aspects, one or more receptors for the biomolecule is immobilized to the capture substrate. In some embodiments, the capture substrate is placed in proximity of the array of nano-wells before, during or after exposure of one or more reagents to the target cells. In some embodiments, measurements of the biomolecules are obtained on a surface of the capture substrate. In some embodiments, the measurements of the biomolecules obtained on the surface of the capture substrate comprise bright field microscopy, fluorescence microscopy, microscopy utilizing a laser source and a photomultiplier tube detector, or a combination thereof. In some embodiments, the biomolecule is a secreted bio-molecule. In some embodiments, the biomolecule comprising an Fc domain. In some embodiments, the biomolecule comprising an Fc domain is an antibody. In some embodiments, the biomolecule is secreted by the target cell. In some embodiments, the biomolecule is a bioparticle. In some embodiments, the biomolecule is presented on the surface of the target cell. In some embodiments, the reference comprises a reference glycan profile. In some embodiments, the reference is a glycan profile reference. In some embodiments, the biomolecule is internal to the target cell.

Described herein are certain aspects for a method for cell line development utilizing mass spectrometry for identification of a biomolecule, comprising: providing a plurality of target cells, an array of nano-wells, one or more reagents and instructions; loading the plurality of target cells into array of nano-wells such that an individual well of the array of nano-wells contains an individual target cell; exposing the plurality of target cells to one or more reagents, wherein each individual target cell can produce a biomolecule; capturing each biomolecule produced by each individual target cell on a capture substrate; wherein the capture substrate is configured to keep each biomolecule produced by each individual target cell in the array of nano-wells spatially distinct and registered to the individual well from which the individual target cell originated; analyzing the biomolecule with mass spectrometry (MS); comparing and the captured biomolecule on the capture substrate to a mass spectrometry-based reference; identifying the captured biomolecule; and selecting a target cell from the individual target cells to be recovered based on the identify of biomolecule produced by the individual target cell. In certain aspects, a time to reach a decision for selecting the individual target cell for recovery does not exceed 3 hours from the initialization of the method. In certain aspects, the method yields clones with differentiated Glycan profile. In certain aspects, the capture substrate is used to create sequential prints. In some embodiments, the capture substrate comprises a MS compatible capture slide. In some embodiments, the mass-spectrometry is MALDI-TOF mass spectrometry. In some embodiments, the mass-spectrometry is MALDI-MSI. In some embodiments, the reference is a reference MS profile. In some embodiments, array of nano-wells comprises individual nano-wells 50 microns in diameter, 100 microns deep. In some embodiments, the individual nano-wells are packed in arrangements comprising hexagonal and square. In some embodiments, the center to center spacing for nano-wells in the array of nano-wells is 100 microns.

Described herein are certain aspects of a method of selecting a target cell based on an aggregation property of a secreted biomolecule, comprising: providing a plurality of cells, an array of nano-wells, and one or more reagents, wherein the plurality of cells comprises a target cell; loading the plurality of cells into the array of nano-wells such that an individual well of the array of nano-wells contains an individual cell; exposing the plurality of cells to one or more reagents, wherein each of the plurality of cells can produce a biomolecule; wherein the target cell can produce a target biomolecule; capturing the biomolecule produced by each individual target cell on a capture substrate; wherein the capture substrate is configured to keep the biomolecule produced by each individual cell in the array of nano-wells spatially distinct and registered to the individual nano-well from which the biomolecule originated; exposing the biomolecule or cell to a reagent or other perturbant that induces aggregation of the biomolecule; determining an aggregation property of the biomolecule to identify the target biomolecule; and selecting the target cell from the plurality of cells to be recovered based on the aggregation property of the target biomolecule. In certain aspects, a time to reach a decision for selecting the individual target cell for recovery does not exceed 3 hours from the initialization of the method. In some embodiments, the method yields clones with less than 7 percent aggregation. In some embodiments, the biomolecule comprises an Fc domain. In some embodiments, the biomolecule comprising an Fc domain is an antibody. In some embodiments, the biomolecule is secreted by the target cell.

Described herein are certain aspects of a method for selecting a target cell; comprising: a) placing a plurality of cells into a plurality of nano-wells, wherein each individual nano-well of a subset of the plurality of nano-wells contains only one individual cell of the plurality of cells; b) exposing at least the subset of the plurality of nano-wells to a condition, wherein the condition is treating with one or more regents, treating with a plurality of secondary cells, applying a membrane to the plurality of nano-wells to form a plurality of membrane-modified nano-wells, providing a suspension of a plurality of secondary cells, contacting a secondary cell-immobilized capture substrate, or a combination thereof, c) detecting a signal from a particular nano-well of the subset of the plurality of nano-wells during or after the exposing, wherein the signal is indicative of the presence of a target cell or a biomolecule produced by the target cell in the particular nano-well of the subset of the plurality of nano-wells; and d) selecting the target cell in the particular nano-well from the plurality of cells based on a pre-determined value of the signal. In some embodiments, the placing, the exposing, the detecting and the selecting is performed on cells under conditions the same as or close to the natural cell culture conditions, thereby leading to more accurate selection of high performing cell for clones when expanded and/or scaled up. In some embodiments, the exposing, the detecting and/or the selecting is performed with little or no perturbation of the cells growing within separate volumes such that the cells remain the same as or close to their shake flask state.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a process of single cell isolation and analysis.

FIG. 2 shows the process of clonal selection.

FIG. 3A shows a system for co-culturing target cells with secondary cells.

FIG. 3B shows a system for co-culturing target cells with secondary cells.

FIG. 3C shows a system for co-culturing target cells with adherent secondary cells.

FIG. 4 shows a block diagram where some of the elements of the integrated system for high-throughput cell line development are shown.

FIG. 5 shows a method of analysis and device for high-throughput cell line development.

FIG. 6 depicts a plate with multiple large wells for sterile sealing of a capture substrate to the bottom of the large wells of the plate.

FIGS. 7A-7E show a system used to contact the nano-well plate with the capture substrate. FIG. 7A shows a configuration of a well on the nano-well plate. FIG. 7B shows another configuration of a well on the nano-well plate. FIG. 7C shows still another configuration of a well on the nano-well plate. FIG. 7D shows one configuration of a well on the nano-well plate. FIG. 7E shows another configuration of a well on the nano-well plate.

FIG. 8 shows a cross section of a large well of a plate (801) made with a hard plastic and a capture substrate (802) made of a soft material.

FIGS. 9A-9D show methods of capture of biomolecules secreted by the target cell.

FIG. 9A shows the capture by a capture substrate. FIG. 9B shows the capture by a beads functionalized with a capture agent. FIG. 9C shows the capture by a capture agent attached to the interior surface of the chamber/well in which the single cell resides. FIG. 9D shows the capture by a capture agent on a hydrogel placed into the chamber/well in which the single cell resides.

FIG. 10A shows one view of an integrated flow cell-SBS format plate for processing multiple samples and arrays of nano-wells. FIG. 10B shows another view of an integrated flow cell-SBS format plate for processing multiple samples and arrays of nano-wells. FIG. 10C shows still another view of an integrated flow cell-SBS format plate for processing multiple samples and arrays of nano-wells.

FIG. 11A shows a label free readout scheme for cell secretion. FIG. 11B shows another label free readout scheme for cell secretion. FIG. 11C shows still another label free readout scheme for cell secretion. FIG. 11D shows another label free readout scheme for cell secretion.

FIG. 11E shows still another label free readout scheme for cell secretion.

FIG. 12 shows a method of automatically analyzing different cells based on their morphologies.

FIG. 13 shows a method for replicating individual cells in the nano-well.

FIG. 14 depicts a method of antibody development and discovery.

FIG. 15 shows a schematic for a live single-cell metabolic assay.

FIG. 16 shows a biosimilar development and clonal selection based on analysis by a lectin affinity binding assay and optical spectroscopy.

FIG. 17 shows a biosimilar development and clonal selection based on key product attribute of glycosylation where analysis is performed by mass spectrometry.

FIG. 18A shows quantification of detected single-cell titers according to reference areas located on a reference device. FIG. 18B shows quantification of detected single-cell titers according to reference areas loaded in each chamber/well. FIG. 18C shows another view of quantification of detected single-cell titers according to reference areas loaded in each chamber/well.

FIGS. 19A-19C show an assay for determining binding interactions at different time intervals.

FIGS. 20A-20B show accurate prediction of single-cell performances. FIG. 20A shows a distribution of predicted single-cell performances. FIG. 20B shows a correlation between single-cells performance and the scale-up performance of these clones on the bioreactor level. CSS stands for Clone Selection Score, which is an arbitrary dimensionless unit.

FIGS. 21A-21B show cell populations experiencing changes and undergoing shifts over time. FIG. 21A shows a cartoon illustration of changes of a cell population at three time points. FIG. 21B shows cell performance diversifies on a cell-by-cell basis.

FIGS. 22A-22C show image confirmation for a single CHO cell before and after picking from a nanowell and the clone that was derived from the single cell on Day 7 and Day 14. FIG. 22A shows a single CHO cell before and after picking from a nanowell. FIG. 22B shows the growth of the single cell into a clone on Day 7. FIG. 22C shows the growth of the single cell into a clone on Day 14.

FIGS. 23A-23C show monoclonality of single-cell recovery. FIG. 23A shows a mixed population of HEK293 cell lines that expressed GFP (6%) and RFP (94%). FIG. 23B shows the growth of a GFP expressing colony on Day 7 and Day 14. FIG. 23C shows the growth of an RFP expressing colony on Day 14.

FIG. 24 shows a workflow on a machine-learning based predictive model for single-cell productivity.

### DETAILED DESCRIPTION

### I. Introduction

The methods and system described herein allow for high-throughput cell line development, providing for rapid identification and characterization of compositions produced by cells. Additionally, the systems and methods disclosed herein provide for rapid identification and characterization of cells as well. Examples of compositions produced by cells include antibodies or cytokines. In addition to providing rapid analysis and characterization, the systems and methods disclosed herein allow for the study and recovery of cells of interest at the single cell level while providing a sterile and gentle environment. In addition to single cells, the platform allows for the growth, study and recovery of small isolated colonies of cells.

In view of the importance of single-cell analysis and selection to genomics, transcriptomics, proteomics, metabolomics, it is desirable to predict with confidence and accuracy that one particular single cell among tens of thousands of candidate cells can produce a high performing clone when the selected single cell is expanded. It is also desirable to complete the single cell to high performing clone selection process within a short time, such as, for example, no more than 48, 24, 18, 12, 6, 5, 4, 3, 2, 1 hour(s).

Previous ways of selection of high performing clone can take days, weeks or months to complete the single cell to high performing clone selection process because the evaluation of performance of clones are done in cultures grown from single cells. For example, a typical clone screening process, such as the traditional microtiter plate-based method of clone generation and growth, may take two to three months. At the first stage, hundreds of pooled, heterogeneous cells are sorted into single-cell cultures through processes such as fluorescence-activated cell sorting (FACS) or limiting dilution. Then these cells are allowed to recover to healthy and stable populations, after which the cells are analyzed, and selected cell populations are transferred to small containers, such as spin tubes, 24-well plates, or 96-deep well plates. These selected and transferred cells are cultured in a cell culture such as a 10-day or 14-day or 10 to 14-day fed batch process. In this small-scale cell culture process, large amount of nutrients are added periodically, and different measurements of cell growth and viability parameters are taken. Hundreds or thousands of these small-scale cell cultures are run in parallel. At the end of the culture (e.g., on the tenth day), the cells are harvested for assays and analysis. Only then the researcher knows which single cell is the “champion” to produce a high performing “champion” clone. During this process, many underperforming cells are allowed to complete the small cell culture step, hence, a wasting of time and resources. It is desirable to complete the cell line selection process at the single cell step without going through the small cell culture step.

Another problem in single cell analysis and cell development is monoclonality. The term “monoclonality” as used herein generally refers to a cell line that originates from a single progenitor or parent cell (single cell)—and is therefore monoclonal. Cell line development and assurance of monoclonality are critical steps in the process of generating biopharmaceutical molecules, such as monoclonal antibodies.

In some cases, the development, scale-up and eventual manufacture of monoclonal antibodies requires optimized, stable, productive cell lines to maximize regulatory compliance, safety, patient benefit and economic viability. Cell lines used for monoclonal antibody production—and any other biologic production—are required by regulatory agencies to have demonstrated evidence of monoclonality. Monoclonality, or lack thereof, can significantly impact product quality, hence, evidencing clonality is a necessary stage in securing regulatory approval.

Described herein are new single cell selection process that can probe candidate single cells on a single cell basis within a short period time to complete the single cell to high performing clone selection process. Features of the new single cell selection process include, but are not limited to, small solution volume for each candidate single cell, fast loading time to deposit candidate single cells from a parent cell culture to the chamber (e.g., a chamber, a reaction chamber, a cell, a container/chamber with an aperture/opening, or a nano-well on a chip), minimal disturbance to the candidate single cells in their respective chambers, keeping the candidate single cells in their respective chambers in an environment similar to that of their parent cell culture, using a substantially planar surface to capture secreted biomolecules from the candidate single cells, perform single cell analysis on the substantially planar surface, completing the selection process without removing the candidate single cells from their respective chambers, completing the selection process without a small-scale culture stage (e.g., 10-day fed batch process), and processing single cell data and predicting scale-up performance of the expanded cell with accuracy.

Described herein are new integrated analytical process that extends these approaches to efficiently and comprehensively evaluate cells from a heterogenous population. The process combines image-based cytometry, microfluidics, and automated micromanipulation to yield multidimensional data on the immunophenotypes of cells, the distribution of isotypes of their secreted biomolecules and the relative affinities of these biomolecules for specific antigens, for thousands of cells in parallel. The approach can be applied to characterize many cell types, including immune cells, or other eukaryotic cells. As an example, suspensions of single cells taken from cancerous tissue of tissue type, e.g., heart, brain, liver, prostate, breast, skin, bone, or colon cancer are compatible with the systems and methods disclosed herein antibody-secreting cells and activated memory B cells from the same individual. This approach can also be applied to characterize and select cells for use in cell therapy. The flexibility and compatibility of the technique with small samples makes this approach a useful complement to existing methods for evaluating humoral responses in humans and should provide a rapid and cost-effective technology for developing new cell lines for therapeutic uses.

Components of the integrated system for high-throughput cell line development are described herein. In some embodiments, an array of nano-wells comprises individual nano-wells for separation of cells from a heterogeneous population. Studying both cell and secreted product is achieved by sealing individual cells within each nano-well with a substrate, where the surface of the substrate facing into the nano-well is functionalized with a capture agent. In the state of the art, processes including (1) the loading of cells, (2) sealing and separating of the substrate to the array of nano-wells, (3) analysis and (4) cell recovery are mainly performed manually. These manual processes are prone to contamination and are cumbersome and time-consuming. Described herein are systems and methods to automate the above processes.

Applications enabled by the integrated systems and methods for high-throughput cell line development described herein include (1) single cell selection based on cell morphology of live cells; (2) a terminal assay using a reference live cell array; (3) antibody discovery and development; (4) a live single-cell metabolic assay; (5) biosimilar development and clonal selection based on key product attributes, such as glycosylation and aggregation. Further described herein are two methods based on glycosylation including a method using a lectin panel assay and a method using mass-spectrometry to identify secreted product.

Characterizing the nature and breadth of the antibody responses generated in humans is important for understanding how vaccines elicit prophylactic protection and for developing new insights to designing effective vaccines against diseases such as HIV, hepatitis C, tuberculosis, and malaria. Despite strong correlates of protection associating humoral responses with common vaccines, it is still unclear how to elicit such responses by rational design. Strategies for reverse engineering of immunogens for vaccines depend on efficient means for identifying and characterizing functional antibodies from infected patients. Furthermore, the enumeration of novel antibodies with useful properties (e.g., broad and potent neutralizing activity) directly from humans may also provide new candidates for therapies and diagnostics.

While darkfield imaging collects scattered light from a defect, brightfield imaging collects reflected light. At a given pixel size, a very small (sub-pixel) imaging system averages everything seen in the pixel, including defect plus background. Brightfield imaging uses a small enough pixel to resolve the edges of the defect and thereby detect a contrast. Darkfield imaging averages everything contained in a pixel, but the background is always black, and even small defects have a tendency to scatter large amounts of light. A flat, opaque defect may scatter very little light in darkfield, but may provide obvious contrast in brightfield. Small, transparent defects may scatter efficiently in darkfield illumination, but may be very difficult, if not impossible, to detect in brightfield. Darkfield imaging is generally useful in detecting defects having specific height, depending upon interaction between illumination with the geometry and effects due to transparent layers on the specimen.

In various embodiments, light sources emitting radiation in the ultraviolet spectrum (wavelengths from about 10 nm to about 400 nm), visible spectrum (wavelengths from about 400 nm to about 700 nm), and/or near-infrared spectrum (wavelengths from about 700 nm to about 3000 nm) are used in the imaging systems and methods provided herein,

As used herein, there term “fluorescence” can refer to forms of luminescence, in particular also phosphorescence. It is a type of non-invasive imaging technique that can help visualize biological processes taking place in a living organism. Images can be produced from a variety of methods including: microscopy, imaging probes, and spectroscopy. Fluorescence itself, is a form of luminescence that results from matter emitting light of a certain wavelength after absorbing electromagnetic radiation. Molecules that re-emit light upon absorption of light are called fluorophores.

The fluorescence excitation radiation can be continuously modulated, that the first image sensor is a solid-state detector which can be driven in a phase-sensitive manner, and that the data supplied by the first image sensor contain pixel-wise phase information of the fluorescence radiation, an endoscopically applicable fluorescence imaging apparatus is inventively provided inexpensive and easy to handle. Continuous modulation of fluorescence excitation radiation can be generated with relatively simple electronic means, and phase-sensitive solid state sensors are simple in construction, easy to handle, and inexpensive to use. With a corresponding modulation frequency, time delays, which are frequent fluorescent substances in the range of lifetimes, can be easily and reliably detected. The pixel-resolved detection and evaluation of the phase information makes it possible to generate an image which represents spatially resolved fluorescence lifetime information. This allows FLIM, for example, to be made available for many diagnostic applications in clinical practice.

Near-infrared (NIR) light can use the NIR wavelengths to detect signals. Hence, NIR light may provide a non-invasive, non-contact and relatively stain insensitive detection method. Broadband light may provide further advantages because carious regions may demonstrate spectral signatures from water absorption and the wavelength dependence of porosity in the scattering of light.

Hyperspectral imaging typically relates to the acquisition of a plurality of images, where each image represents a narrow spectral band collected over a continuous spectral range. For example, a hyperspectral imaging system may acquire 15 images, where each image represents light within a different spectral band. Acquiring these images typically entails taking a sequence of photographs of the desired object, and subsequently processing the multiple images to generate the desired hyperspectral image. In order for the images to be useful, however, they must be substantially similar in composition and orientation. For example, the subject of the images must be positioned substantially identically in each frame in order for the images to be combinable into a useful hyperspectral image. Because images are captured sequentially (e.g., one after another), it can be very difficult to ensure that all of the images are properly aligned. This can be especially difficult in the medical context, where a clinician is capturing images of a patient who may move, or who may be positioned in a way that makes imaging the subject area difficult or cumbersome.

The methods and systems described herein have multiple advantages over previous methods such as FACs. The methods are well suited for analyzing single cells from a small population of total cells (10⁵ cells or less). Second, the methods described herein have better sensitivity for rare cells and allow for characterization of antibodies or other biomolecules produced by the cells described herein, compared to flow cytometry. Third, the methods described herein allow for physical separation of the antibody or other biomolecule produced from the cells to allow multiplexed analysis of the antibodies without damage or minimum perturbation to the source cells. Further, the methods and systems described herein can be used to identify and select other useful cell types.

### II. Methods

### A. An Integrated Method for High-Throughput Cell Line Development

### Method Overview

In certain aspects, disclosed herein is a method for high-throughput cell line development, comprising: a) providing a plurality of target cells, an array of nanoliter volume, nano-wells, one or more reagents and instructions; b) loading the plurality of target cells into an array of nano-wells; c) exposing the plurality of target cells to one or more reagents; d) obtaining measurements of individual target cells and measurements of individual articles associated with the individual target cells; e) selecting a target cell from the individual target cells to be recovered based on predetermined values of the measurements; wherein a time to reach a decision for selecting the target cell for recovery does not exceed 3 hours from the initialization of the method; and wherein the method yields clones with a mean productivity of 5 grams per liter. In certain embodiments, the target cell that is recovered is live. Also, in certain embodiments, disclosed herein is a method for recovering cells utilizing the selection process described herein.

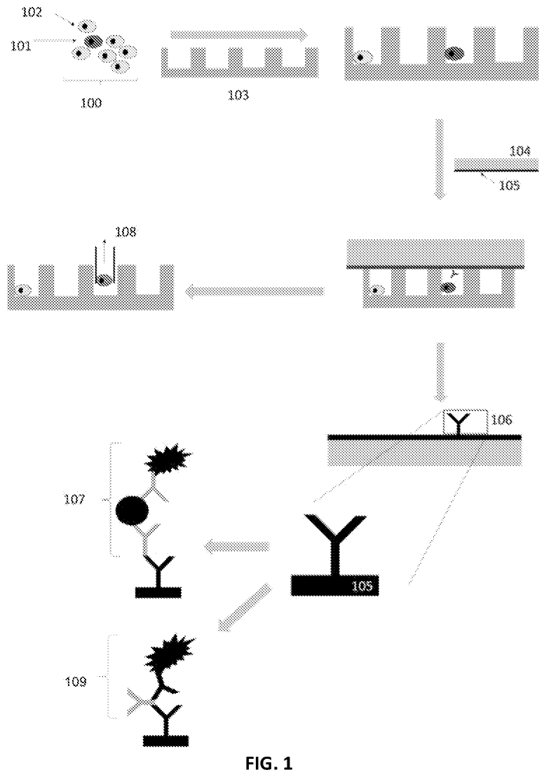

In certain embodiments, disclosed herein is a process of single cell isolation and analysis. As seen in FIG. 1, a heterogenous cell population (100) contains cells of interest (101) as well as other cells (102). The heterogenous cell population is loaded into the nano-well chip (103). Single cells are isolated in an individual nano-wells. In some embodiments, a capture substrate (104) is used in secretion profiling, imaging, and subsequent analytics of the single cells. A capture layer (105) captures the secreted biomolecule (106). Assay reagents (107, 109) are added to the system. After analysis, in some embodiments, an identified single cell is recovered (108) from the nano-well chip (103).

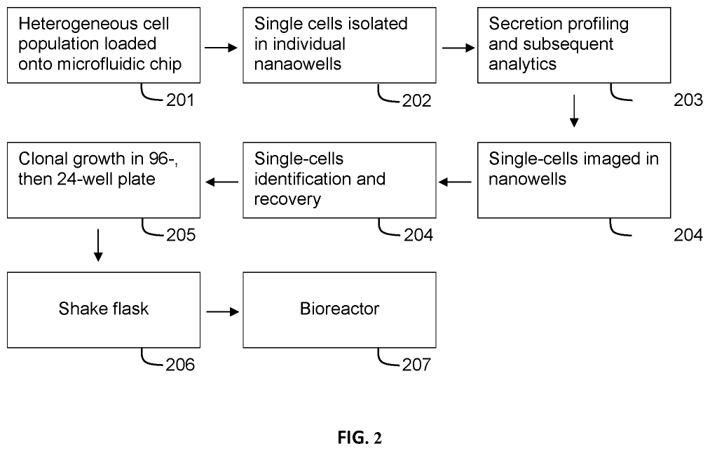

In some embodiments, as seen in FIG. 2, described herein is method of using the single cell selection method comprising at least one step of Steps 201-208. In Step 201 a transfected pool of heterogeneous cells are loaded onto a microfluidic nano-well chip. In Step 202, single cells are isolated in individual nanowells of the microfluidic ship. In Step 203, secreted molecules from the single cells in individual nanowells are profiled; and the secretion profiling and subsequent analysis is performed. In Step 204, images of single cells in nanowells are taken and analyzed. In Step 205 selected single cells are identified and recovered. After the single cell selection process is completed, in Step 206, the selected single cells are grown as individual clones 6, 12, 24, 96 and 384 well plates, followed by expansion of the cell lines in cell culture either in a shake flask (Step 207) or in a bioreactor (Step 208) for confirmation and production.

### Target Cells

In certain embodiments, the target cell is a T cell, a B cell, a plasma cell, antibody secreting cells (ASCs), an antigen presenting cell, a hybridoma, an immune cell, a stem cell, an induced pluripotent stem cell (IPSC), or an engineered cell. In certain embodiments, the engineered cell is a CHO cell, a HEK 293 cell, a murine NSO cell, CAP cell, AGE cell, SP2/0, BHK21, HKB-11, HuH-7, C127, TKT, HT-1080 cell, a HELA cell, engineered B cell, engineered NK cell, engineered T cell such as CAR T cell, engineered dendritic cell, an engineered antigen presenting cell, or differentiated IPSC. In certain embodiments, the cell is a lymphocyte, leukocytes tumor cell, stromal cell, neuronal cell, stem cell, gametes such as sperm cell and ova cell, or an embryo. In certain embodiments, the cell is a primary cell, a cell line, an eukaryotic cell, prokaryotic cell, a yeast cell, a bacterial cell, an E. coli cell or a P. pastoris cell.

In some embodiments, the transgene encodes a cellular receptor. In some embodiments, the cellular receptor is a receptor found on the surface of an immune cell. In some embodiments, the cellular receptor is engineered to target or bind to a specific antigen. In some embodiments, the antigen is a marker for an infection, autoimmune disease or cancer described herein. In some embodiments, the antigen comprises a viral antigen, self-antigen or tumor antigen. In some embodiments, the cellular receptor is a pattern recognition receptor, such as a Toll-like receptor, a C-type lectin receptor, a NOD-like receptor, or a RIG-I-like receptor. In some embodiments, the transgene encodes a killer activated receptor or a killer inhibitor receptor. In some embodiments, the transgene encodes a complement receptor. In some embodiments, the transgene encodes an Fc receptor. In some embodiments, the transgene encodes a B cell receptor. The B-cell receptor may comprise an immunoglobulin selected from the group consisting of IgD, IgM, IgA, IgG, and IgE. In some embodiments, the transgene encodes a T cell receptor.

In some embodiments, the transgene encodes a chimeric antigen receptor. The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. In some embodiments, CARs are employed to impart the specificity of a monoclonal antibody onto a T cell, thereby allowing a large number of specific T cells to be generated, for example, for use in adoptive cell therapy. In some embodiments, CARs direct specificity of the cell to a tumor associated antigen. In some embodiments, CARs comprise an intracellular activation domain, a transmembrane domain, and an extracellular domain comprising a tumor associated antigen binding region. In some embodiments, CARs comprise fusions of single-chain variable fragments (scFv) derived from monoclonal antibodies, fused to a transmembrane domain and endodomain. In some embodiments, the specificity of other CAR designs is derived from ligands of receptors (e.g., peptides) or from Dectins. In some embodiments, CARs comprise domains for additional co-stimulatory signaling, such as CD3, FcR, CD27, CD28, CD137, DAP10, and/or OX40.

In some embodiments, the transgene encodes a major histocompatibility complex (MHC). In some embodiments, the MHC molecule is a MHC class I molecule or a MHC class II molecule. In some embodiments, MHC molecule is a human leukocyte antigen (HLA). In some embodiments, the MHC molecule is selected from the group consisting of HLA-A, HLA-B, HLA-C, HLA-DP, HLA-DM, HLA-DOA, HLA-DOB, HLA-DQ, and HLA-DR.

In some embodiments, the transgene encodes an antibody.

Article Associated with Target Cells

In certain embodiments, the target cell produces an individual article. In some embodiments, the article is a biomolecule. In certain embodiments, the biomolecule is a binding molecule. In certain embodiments, the biomolecule is a biomolecule comprising an Fc domain. In certain embodiments, the biomolecule comprising an Fc domain is an antibody, an antivenom, or an antitoxin. In certain embodiments, the article is an antibody mimetic. In certain embodiments, the antibody mimetic is an affibody, an adnectin, an affilin, an affimer, an affatin, an alphabody, an anticalin, an aptamer, an atrimer, an avimer, a fynomer, a DARPin, an armadillo repeat protein, a Kunit domain inhibitor molecule, a knottin molecule, a designated ankyrin repeat molecule, a monobody or a nanofitin. In certain embodiments, the article is a C-type lectin. In certain embodiments, the article is a bioparticle. In some embodiments, the bioparticle comprise cell secreted vesicles further comprising microparticles, ectosomes, shedding vesicles, micro-vesicles, extracellular vesicles, or exosomes. In certain aspects the bioparticle is a virus.

In certain embodiments, the article is secreted by the target cell. In certain embodiments, the article is presented on the surface of the target cell. In certain embodiments, the article is internal to the target cell.

In certain embodiments, the biomolecule is encoded by a heterologous gene.

In certain embodiments, the biomolecule binds to one or more antigens that are markers for infection. In certain embodiments, the infection is a viral infection, a parasitic infection, a bacterial infection, or a bioweapon-based infection.

In certain embodiments, the biomolecule binds to one or more antigens that are markers for autoimmune disease. Autoimmune disorders include diabetes mellitus (diabetes melitus), transplant rejection, multiple sclerosis, premature ovarian dysfunction, scleroderm, Sjogren's disease, lupus, vilelego, alopecia (baldness), Multi-glandular dysfunction, Graves' disease, hypothyroidism, polymyosititis, pemphigus, Crohn's disease, colitis, autoimmune hepatitis, hypopituitarism, myocarditis, Addison's disease, autoimmune skin disease, uveititis, pernicious anemia, hypoparathyroidism, and/or rheumatoid arthritis.

In certain embodiments, the biomolecule binds to one or more antigens that are markers for cancer. Some examples of such cancers include, but are not limited to: Adrenocortical cancer; Bladder cancer; Breast cancer; Breast cancer, Breast duct; Breast cancer, Invasive duct; Breast-ovarian cancer; Burkitt lymphoma; cervical cancer; colon adenoma; colon cancer; colon cancer, hereditary non-polyposis, type 1; colon cancer, hereditary non-polyposis, type 2; colon cancer, hereditary non-polyposis, type 3; colon cancer, Hereditary nonpolyposis, type 6; colon cancer, hereditary nonpolyposis, type 7; elevated dermal fibrosarcoma; endometrial cancer; esophageal cancer; gastric cancer, fibrosarcoma, glioblastoma multiforme; glomus tumor, multiple types Hepatoblastoma; hepatocellular carcinoma; primary hepatocellular carcinoma; leukemia, acute lymphoblastic; leukemia, acute myeloid; leukemia, acute myeloid, with eosinophilia; leukemia, acute nonlymphoid; leukemia Chronic myelogenous; Li Fraumeni syndrome; Liposarcoma, lung cancer; lung cancer, small cell; lymphoma, non-Hodgkin; Lynch cancer familial syndrome II; male germ cell tumor; mast cell leukemia; thyroid medullary carcinoma; medulloblastoma; melanoma; meningioma; Tumor; Myeloid malignancy, predisposition to it; Myxosarcoma, neuroblastoma; Osteosarcoma; Ovarian cancer; Ovarian cancer, Serous; Ovarian malignant tumor; Ovarian chordoma; Pancreatic cancer; Pancreatic endocrine tumor; Tumor, familial non-chromophilic; hair matrix; pituitary tumor, invasive; prostate adenocarcinoma; prostate cancer; renal cell carcinoma, papillary, familial and sporadic; retinoblastoma; rhabdoid predisposition syndrome, family Rhabdoid tumor; rhabdomyosarcoma; small cell lung cancer; soft tissue sarcoma, squamous cell carcinoma, head and neck; T-cell acute lymphoblastic leukemia; Turcot syndrome with glioblastoma; thickening/esophageal cancer; Cancer; colorectal cancer; lung cancer; prostate cancer; skin cancer; bone Solid tumor/malignant tumor; mucinous and round cell carcinoma; locally advanced tumor; human soft tissue cancer; cancer metastasis; squamous cell carcinoma; squamous cell carcinoma of the esophagus; oral cancer; cutaneous T cell lymphoma; Hodgkin lymphoma; Adrenal cortex cancer; ACTH-producing tumor; Non-small cell carcinoma; Gastrointestinal cancer; Urogenital cancer; Female genital malignant tumor; Male genital malignant tumor; Kidney cancer; Brain tumor; Bone cancer; Skin cancer; Thyroid cancer; Peritoneal exudate; malignant pleural effusion; mesothelioma; Wilms tumor; gallbladder cancer; chorionic tumor; angiodermocytoma; Kaposi's sarcoma and liver cancer. In some embodiments, the one or more antigens are markers for neurological disorders. In some embodiments such neurological disorders comprise Alzheimer's Disease, Amyloid Neuropathy, Amyotrophic Lateral Sclerosis (ALS), Ataxia, Bell's Palsy, Brain Tumors, Cerebral Aneurysm, Epilepsy, or Seizures. In some embodiments, the one or more antigens are markers for metabolic disorders comprising: Familial hypercholesterolemia, Gaucher disease, Hunter syndrome, Krabbe disease, Metachromatic leukodystrophy, Mitochondrial encephalopathy, lactic acidosis, stroke-like episodes (MELAS); Niemann-Pick, Phenylketonuria (PKU), Porphyria, Tay-Sachs disease or Wilson's disease. In some embodiments, the one or more antigens are markers for cardiovascular diseases comprising stroke, vascular disease, arrhythmias, aorta disease, Marfan syndrome, congenital heart disease, coronary artery disease, deep vein thrombosis and pulmonary embolism, heart attack, heart failure, heart muscle disease, heart valve disease peripheral vascular disease and rheumatic heart disease.

In certain embodiments, the bacterial infection is characterized by extracellular bacteria. In certain embodiments, the bacterial infection is characterized by intracellular bacteria. In some embodiments, the bacterial infection is characterized by gram negative bacteria. In some embodiments, the bacterial infection is characterized by gram positive bacteria. In some embodiments, the bacterial infection is characterized by bacteria belonging to one or more of the following bacterial genera comprising: Klebsiella, Clostridium, Naegleria, Acinetobacter, Bacteroides, Borrelia, Brucella, Ehrlichia, Escherichia, Haemophilus, Fusobacterium, Leptospira, Listeria, Mycobacterium, Mycoplasma, Neisseria, Nocardia, Prevotella, Rickettsia, Staphylococcus, Streptococcus, and Treponema. In certain embodiments, the bacterial infection is characterized by bacteria including: Klebsiella pneumoniae, Clostridium difficile, Naegleria fowleri, Acinetobacter baumannii, Borrelia burgdorferi, Escheririchia coli, Haemophilus influenza, Listeria monocytogenes, Mycobacterium tuberculosis, Neisseria meningitides, Nocardia asteroids, Staphylococcus aureus, Streptococcus agalactiae, Streptococcus intermedius, Streptococcus pneumoniae, and Treponema pallidum.

In certain embodiments, the infection is a viral infection. In certain embodiments, the virus is a DNA virus or an RNA virus. In certain embodiments, the pathogenic infection is characterized by a virus belonging to one of the following virial families including: Bunyaviridae, Flaviviridae, Herpesviridae, Orthomyxoviridae, Papovaviridae, Paramyxoviridae, Picornaviridae, Togaviridae, Retroviridae, and Rhabdoviridae. In certain embodiments, the pathogenic infection is characterized by a virus including: Herpes simplex virus (HSV), varicella zoster virus, cytomegalovirus (CMV), Epstein-Barr virus (EBV), Eastern equine encephalitis (EEE), western equine encephalitis (WEE), rubella virus, poliovirus, coxsackievirus, an enterovirus, St. Louis encephalitis (SLE), Japanese encephalitis, rubeola (measles) virus, mumps virus, California encephalitis, LaCrosse virus, human immunodeficiency virus (HIV), rabies virus, and Influenza A virus. In certain embodiments, the viral infection is a corona virus, such as SARS-CoV-2. In certain embodiments, the infection is known to cause pandemic levels of infection.

In certain embodiments, the infection is characterized by a parasite. In certain embodiments, the parasite is a helminth or a protozoan. In certain embodiments, the pathogenic infection is characterized by a parasite belonging to one of the following parasite genera comprising: Angiostrongylus, Cysticercus, Echinococcus, Entamoeba, Gnathostoma, Paragnoimus, Plasmodium, Taenia, Toxoplasma, Trypanosoma, and Schistosoma. In certain embodiments, the pathogenic infection is characterized by a parasite including: Angiostrongylus cantonesis, Entamoeba histolytica, Gnathostoma spinigerum, Taenia solium, Toxoplasma gondii, and Trypanosoma cruzi.

In certain embodiments, the infection is a fungal infection. In certain embodiments, the infection is characterized by a fungus belonging to one of the following fungal genera comprising: Aspergillus, Bipolaris, Blastomyces, Candida, Cryptococcus, Coccidioides, Curvularia, Exophiala, Histoplasma, Mucorales, Ochroconis, Pseudallescheria, Ramichloridium, Sporothrix, and Zygomyctes. In certain embodiments, the infection is characterized by a fungus including Blastomyces dermatitidis, Candida albicans, Coccidioides immitis, Cryptococcus gattii, Cryptococcus neoformans, Curvalaria pallescens, Exophiala dermatitidis, Histoplasma capsulatum, Onchroconis gallopava, Psudallescheria boydii, Ramichloridium mackenziei, and Sporothrix schenckii.

In certain embodiments, one or more reagents comprise one or more secondary cell, reporter cell, perturbing cell, one or more cellular factors, media, antigen, secondary binding molecule, labeling molecule, or a combination thereof. In some embodiments, one or more cellular factors are capable of modifying a cell in terms of parameters comprising growth or perturbations.

The methods and systems described herein use an array of nano-wells to isolate individual cells to a few cells in individual wells.

In certain aspects, disclosed herein is a method comprising loading a plurality of target cells into an array of nano-wells. In certain embodiments, the number of cells per a well of the array of nano-wells is about 0 to about 50. In certain embodiments, the number of cells per a well of the array of nano-wells is about 0 to about 1, about 0 to about 5, about 0 to about 10, about 0 to about 50, about 1 to about 5, about 1 to about 10, about 1 to about 50, about 5 to about 10, about 5 to about 50, or about 10 to about 50. In certain embodiments, the number of cells per a well of the array of nano-wells is about 0, about 1, about 5, about 10, or about 50. In certain embodiments, the number of cells per a well of the array of nano-wells is at least about 0, about 1, about 5, or about 10. In certain embodiments, the number of cells per a well of the array of nano-wells is at most about 1, about 5, about 10, or about 50.

In certain embodiments, the number of cells present on an array of nano-wells is 100 to 10,000,000. In certain embodiments, the number of cells present on an array of nano-wells is 100 to 1,000, 100 to 10,000, 100 to 100,000, 100 to 10,000,000, 1,000 to 10,000, 1,000 to 100,000, 1,000 to 10,000,000, 10,000 to 100,000, 10,000 to 10,000,000, or 100,000 to 10,000,000. In certain embodiments, the number of cells present on an array of nano-wells is 100, 1,000, 10,000, 100,000, or 10,000,000. In certain embodiments, the number of cells present on an array of nano-wells is at least 100, 1,000, 10,000, or 100,000. In certain embodiments, the number of cells present on an array of nano-wells is at most 1,000, 10,000, 100,000, or 10,000,000.

In certain embodiments, the volume of target cells in a sample does not exceed 0.1 milliters to 0.5 milliters. In certain embodiments, the volume of target cells in a sample does not exceed 0.1 milliters to 0.2 milliters, 0.1 milliters to 0.5 milliters, or 0.2 milliters to 0.5 milliters. In certain embodiments, the volume of target cells in a sample does not exceed 0.1 milliters, 0.2 milliters, or 0.5 milliters. In certain embodiments, the volume of target cells in a sample does not exceed at least 0.1 milliters, or 0.2 milliters. In certain embodiments, the volume of target cells in a sample does not exceed at most 0.2 milliters, or 0.5 milliters.

In certain embodiments, the number of target cells in a sample does not exceed 1,000 per milliliter to 200,000 per milliliter. In certain embodiments, the number of target cells in a sample does not exceed 1,000 per milliliter to 2,000 per milliliter, 1,000 per milliliter to 10,000 per milliliter, 1,000 per milliliter to 20,000 per milliliter, 1,000 per milliliter to 200,000 per milliliter, 2,000 per milliliter to 10,000 per milliliter, 2,000 per milliliter to 20,000 per milliliter, 2,000 per milliliter to 200,000 per milliliter, 10,000 per milliliter to 20,000 per milliliter, 10,000 per milliliter to 200,000 per milliliter, or 20,000 per milliliter to 200,000 per milliliter. In certain embodiments, the number of target cells in a sample does not exceed 1,000 per milliliter, 2,000 per milliliter, 10,000 per milliliter, 20,000 per milliliter, or 200,000 per milliliter. In certain embodiments, the number of target cells in a sample does not exceed at least 1,000 per milliliter, 2,000 per milliliter, 10,000 per milliliter, or 20,000 per milliliter. In certain embodiments, the number of target cells in a sample does not exceed at most 2,000 per milliliter, 10,000 per milliliter, 20,000 per milliliter, or 200,000 per milliliter.

In certain embodiments, described herein is a method for predicting from about 100 to about 1,000,000 heterogenous cells which cell or cells can produce high performing clones when the cell or cells are expanded. In certain embodiments, described herein is a method for predicting from about 100 to 10,000, from about 1,000 to 10,000, from about 1,000 to 100,000, from about 10,000 to 100,000, from about 10,000 to 1,000,000, from about 100,000 to 1,000,000 heterogenous cells which cell or cells can produce high performing clones when the cell or cells are expanded. In certain embodiments, selecting the cell or cells from the about 10,000 to about 1,000,000 heterogenous cells can be completed in no more than 10, 9, 8, 7, 6, 5, 4, 3, 2, or 1 hour. In certain embodiments, selecting the cell or cells from about 10,000 to about 1,000,000 heterogenous cells is completed from 10 to 9 hours, from 9 to 8 hours, from 8 to 7 hours, from 7 to 6 hours, from 6 to 5 hours, from 5 to 4 hours, from 4 to 3 hours, from 3 to 2 hours, from 2 to 1 hour(s), from 60 to 30 minutes, and from 30 to 1 minute(s).

In certain embodiments, the analysis to make the prediction and selection is performed by probing the cell populations on a cell-by-cell basis faster than the traditional cell line development methods. In certain embodiments, going from heterogenous cells in culture to measuring signals from probing individual cells to making the prediction and selection of cells for high performing clones is completed from 4 days to 5 minutes, from 3 days to 5 minutes, from 2 days to 5 minutes, from 1 day to 5 minutes, from 6 hours to 5 minutes, from 5 hours to 5 minutes, from 4 hours to 5 minutes, from 3 hours to 5 minutes, from 2 hours to 5 minutes, from 1 hour to 5 minutes, from 30 to 5 minutes, from 20 to 5 minutes, from 15 to 5 minutes, or from 10 to 5 minutes.

In certain embodiments, the analysis to make the prediction and selection of cells for high performing clones is performed within separate volumes from pico liter to nano liter scales such that the concentration of secreted molecules from cells within such separate volumes reaches the detectable level faster than the traditional and other cell line development methods. In certain embodiments, the differences of the concentrations of secreted molecules within such separate volumes (comprising high performing clones or low performing clones) reach the detectable level faster than the traditional and other cell line development methods. In certain embodiments, loading cells from the about 100 to about 1,000,000 heterogenous cells into a plurality of wells on a chip is completed in no more than 30, 29, 28, 27, 26, 25, 24, 23, 21, 20, 19, 18, 17, 16, 15, 14, 12, 11, 10, 9, 8, 7, 6, 5, 4, 3, 2, or 1 minute(s). In certain embodiments, selecting the cell or cells from the about 100 to about 1,000,000 heterogenous cells can be completed from 30 to 10 minutes, from 10 to 9 minutes, from 9 to 8 minutes, from 8 to 7 minutes, from 7 to 6 minutes, from 6 to 5 minutes, from 5 to 4 minutes, from 4 to 3 minutes, from 3 to 2 minutes, from 2 to 1 minute(s), from 60 to 30 seconds, and from 30 to 1 second(s). In certain embodiments, analysis to make the prediction and selection of cells in wells for high performing clones is performed while the cells in the wells receive reduced or little perturbation when the signals are taken. In certain embodiments, the perturbation is less when compared with the traditional or other cell line development methods. In certain embodiments, the perturbation comprises biological, chemical or mechanical perturbations with regard to the cells or the solution/environment of the cells. In certain embodiments, the perturbation comprises removing cells during recovery or during the analysis of cells in different separate volumes. In certain embodiments, the time counting from removing heterogenous cells from the original culture flask till the selection of cells for high performing clones is shorter than the time in traditional or other cell line development methods. In some embodiments, during the analysis or until the end of the analysis/selection the cells in wells remain the same as or similar to cells in their shake flask state when the heterogenous cells are removed from the shake flask (or other containers).

In some embodiments, the secreted molecules from cells in wells are captured on a planar surface for analysis. In some embodiments, the planar surface on which the secreted molecules are captured coupled with the small volume of the solution sealed in each well by the planar surface leads to (1) higher precision for measurements of the captured secreted molecules and (2) low detection limit of the detection system when compared with traditional or other cell line development methods. In some embodiments, the combination of the above factors, including, for example, selection from a larger pool of heterogeneous cells, smaller volume in wells, single cell in a well, shorter time to make the selection starting from the shake flask state, less perturbation of the cells in each well, planar surface to capture the secreted molecules from the cells in a well, higher precision for measurement of the captured secreted molecules and low detection limit, leads to more reliable and faster prediction of (1) properties of the expanded clonal populations based on the analysis of the single-cell chemical/biological behavior of the corresponding cells in a well, and/or (2) how each cell performs in the corresponding expanded clonal populations, including, for example, the production of antibody, biosimilar, virus, other proteins, nucleotides, and metabolites. In some embodiments, a high predictive correlation between single-cell behavior and scale up bioreactor behavior is found. In some embodiments, the high predictive correlation is found in CHO cells producing a biosimilar. In some embodiments, the predictive correlation between single-cell behavior and scale-up bioreactor behavior with regard to production performance is better than that of a traditional or other cell line development methods. In some embodiments, fewer clones are scaled up to confirm the finding of a high performing clone when compared with a traditional cell line development method, which expanded individual cells into clones for evaluation of desired properties to make selection of the better clone. Thus, the traditional or other cell line development methods are more costly, more time-consuming, less accurate, and/or more unpredictable than the disclosed single-cell methods of the present disclosure. In some embodiments, the disclosed analysis/selection is performed on cells under conditions the same as or close to the natural cell culture conditions, thereby leading to more accurate selection of high performing cell for clones when expanded and/or scaled up.

### B. Co-Culture Methods

In certain embodiments, described herein is a method for high-throughput cell line development comprising exposing a plurality of target cells to one or more reagent. In certain embodiments, the one or more reagents comprise one or more secondary cells, one or more factors, media, or a combination thereof. In certain embodiments, the one or more factors are capable of modifying a cell in terms of growth, perturbations, secretion or motility.

In certain aspects, disclosed herein is a method for isolated co-culture utilizing a secondary cell suspension, comprising; a) providing a plurality of target cells; b) providing an array of nano-wells; c) loading individual cells of the plurality of target cells into the array of nano-wells; d) applying a membrane to the array of nano-wells to form a membrane-modified array of nano-wells; and e) providing a suspension of a plurality of secondary cells, one or more reagents, or a combination thereof, near or in contact with the membrane-modified array of nano-wells. In certain aspects, disclosed herein is a method for isolated co-culture utilizing a secondary cell immobilized-substrate, comprising; a) providing a plurality of target cells; b) providing an array of nano-wells; c) providing a plurality of secondary cells immobilized to a substrate; d) loading individual cells of the plurality of target cells into the array of nano-wells; e) applying a membrane to the array of nano-wells to form a membrane-modified array of nano-wells; and f) simultaneously contacting the membrane-modified array of nano-wells and the secondary cell-immobilized substrate with one or more reagents. In certain embodiments, the plurality of secondary cells reside in a chamber that is fluidically connected to a flow cell containing the membrane-modified array of nano-wells. In certain embodiments, the flow rate of the secondary cell suspension, the one or more reagents, or a combination thereof is equal to or greater than about 0 milliliters per minute.

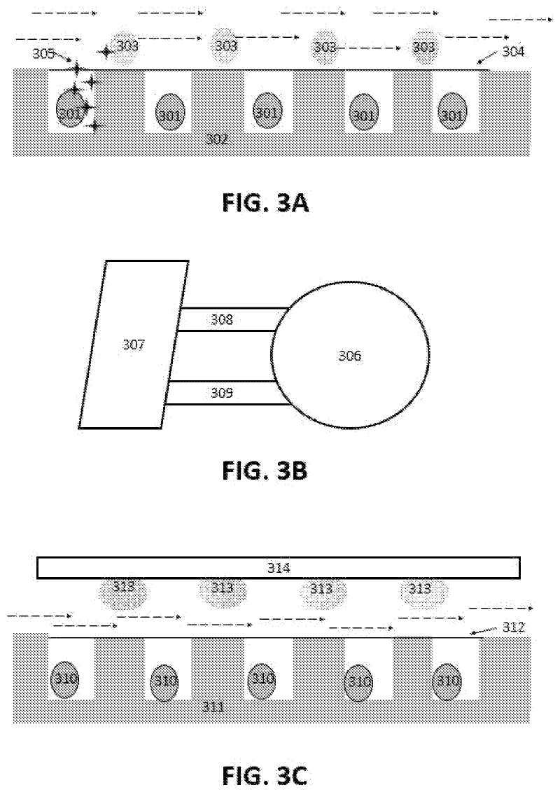

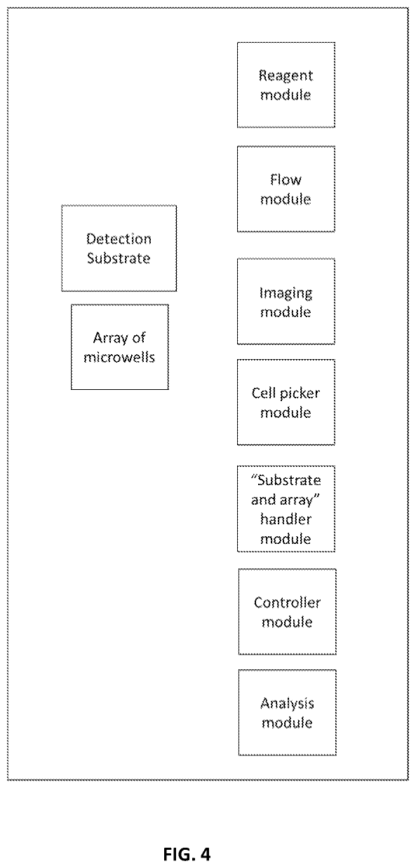

In some embodiments as described herein and shown in FIG. 3A, is a system for co-culturing target cells with secondary cells. In some embodiments, target cells (301) are located in nano-wells (302) covered by a semi-permeable membrane (304) to prevent cell mixing. In certain aspects, secondary cells (303) are flowed across the plate. In some embodiments, factors (305) are transferred from the secondary cell to the target cell and/or vice versa.

In some embodiments, target cells and secondary cells are located in nano-wells. In certain aspects, a secondary cell is located in the same nano-well as a target cell. In some embodiments, a secondary cell is located in a nano-well adjacent to a nano-well containing a target cell. In some embodiments, a secondary cell comprises a feature to distinguish it from the target cell. In certain aspects, the feature is a cell stain or a fluorescent marker.

In some embodiments, as shown in FIG. 3B is a system for culturing target cells with secondary cells. In these instances, cells and media flow from a culture dish with secondary cells (306) into a nano-well chip (307) through an inlet (308) and an outlet (309) channel.

In some embodiments, as shown in FIG. 3C is a system for co-culturing target cells with adherent secondary cells. In these embodiments, target cells (310) are located in nano-wells (311) covered by a semi-permeable membrane (312) to prevent cell mixing. In some embodiments, adherent secondary cells (313) are attached to a substrate (314). In some embodiments, adherent secondary cells are attached to a capture substrate.

In certain aspects, disclosed herein is a method for co-culture utilizing a not-isolated secondary cell suspension. In some embodiments, secondary cells are cultured on the nano-well chip. In some embodiments, secondary cells are distinguished from target cells through a stain. In some embodiments, secondary cells are distinguished by imaging. In some embodiments, zero or more secondary cells are in the same nano-well as the target cell or are in a separate nano-well on the same chip. In some embodiments, secondary cells are feeder cells. In some embodiments, feeder cells are reporter cell lines, or other cells lines to utilize, or probe, cell-cell interactions between the feeder cells and the target cells. In some embodiments, cell-cell and cell-secreted biomolecule-cell interactions are detected and quantified.

### C. Label-Free Detection

In certain embodiments disclosed herein, the capture substrate comprises a sensing surface. In some embodiments, the array of nano-wells comprises the sensing surface. In some embodiments, the sensing surface comprises a substrate. In some embodiments, the substrate comprises a glass substrate. In other embodiments, the substrate comprises a plastic substrate.

In some embodiments the substrate comprises a semiconductor. In some embodiments, the substrate comprises a layered semiconductor. In some embodiments, the substrate comprises a soft material. In some instances, the sensing surface is configured for reflection mode imaging for real-time or endpoint detection of binding interactions on the sensing surface. In certain instances, the sensing surface is configured for surface plasmon resonance detection of the articles. In other embodiments, the sensing surface is configured for interferometric detection of the articles. In certain instances, the sensing surface is configured for whispering gallery mode detection of the articles.

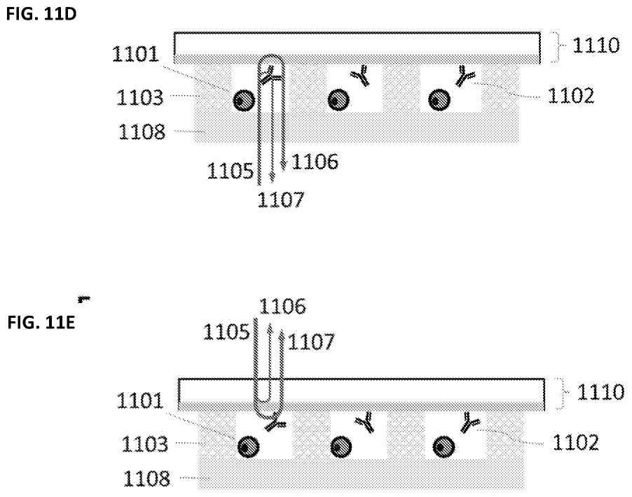

In some embodiments, as seen in FIG. 11A, readout of cell secretion is label free. In certain aspects, secreted analytes (1102) of live single cells (1101) are captured on a layered capture substrate well array bottom (1104). In some embodiments, white light (1105) is illuminated onto the bottom of the layered well array bottom (1104). In some embodiments, the incident light (1105) is reflected off of a reference layer (1106) and the biolayer composed of secretions (1107) is readout using interferometric imaging, due to the interference of the light reflected from the reference layer and the accumulated or accumulating biolayer. In some embodiments, data is collected continuously throughout the experiment. In some embodiments, data is collected at the end of the experiment as an end-point readout. In some embodiments, the wells are not sealed. In some embodiments, as shown in FIG. 11B, readout is performed from the top, where light (1105) is reflected off of the bottom of the wells (1108). In some embodiments, as shown in FIG. 11C, secreted analytes are captured on fiber or lens array dipped inside the well array. In some embodiments, readout is through the fiber or lens array with an internal reference layer (1109) via interferometric readout. Illumination light (white light, LED, laser) reflected from the reference layer in the fiber or lens elements and from the accumulated biolayer interference layer. In these embodiments, readout is continuous or performed at the end of the experiment. In some embodiments, the nano-wells are unsealed by a substrate. In some embodiments, the nano-wells are sealed by a substrate, as shown in FIG. 11D. In some embodiments, secreted analytes are captured on a layered substrate lid (1110).

In some embodiments, readout is through the bottom of the array of wells (1108) via interferometric imaging. In some embodiments, illumination light is reflected from a reference layer in the substrate (1110) and from the accumulated biolayer interference layer (1007). In some embodiments, the illumination source is a white light source, a light emitting diode (LED) source or a laser source. In these embodiments, readout is continuous or end-point readout. In some embodiments, readout is the same as in FIG. 11D, except readout is from the top and through the substrate (1110) as seen in FIG. 11E.

### D. Single-Cell Selection Based on Cell Morphology of Live Cells

In certain aspect, the methods disclosed herein comprise obtaining a measurement of individual live target cells. In certain embodiments, the methods disclosed herein comprise obtaining a measurement associated with an individual article associated with the individual target cells.

In certain embodiments, the measurements of individual target cells comprise characterizations of cellular objects, through segmentation or without segmentation, such as morphology, size, texture of nucleolus, endoplasmic reticulum, nucleoli, cytoplasmic RNA, actin, cytoskeleton, golgi, plasma membrane, mitochondria and other organelles or cell components or a combination thereof. In certain embodiments, the direct measurements comprise bright field microscopy. In certain embodiments, the direct measurements comprise fluorescence microscopy. In certain embodiments, direct measurements comprise microscopy measurements utilizing a laser source and a photomultiplier tube for detection. In certain embodiments, the measurements of individual target cells is done using a stain. In certain embodiments, the measurements of individual target cells is done without a stain. In certain embodiments, the measurements are performed on live individual target cells. In certain embodiments, the measurements are performed on fixed individual target cells. In certain embodiments, data from the measurements of individual target cells is used to create a training set to predict cellular function. In certain embodiments, the target cell is a live cell.

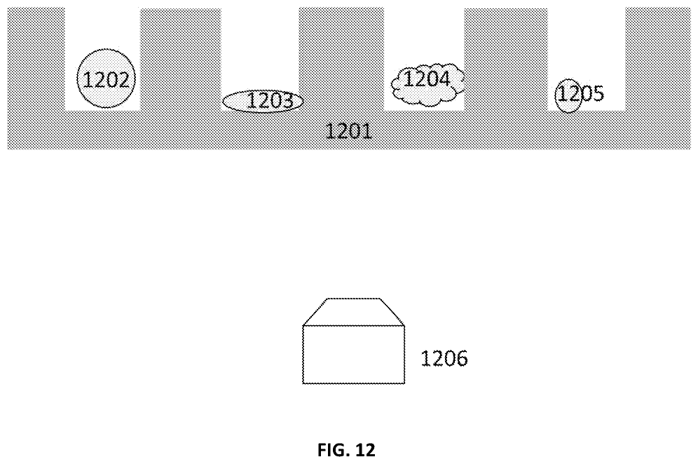

Described herein, as shown in FIG. 12 are methods of automatically analyzing different cells. In some embodiments, an array of nano-wells (1201) comprises a plurality of nanoliter or sub-nanoliter volume wells, each containing an individual cell or few cells (1202-1205). In certain circumstances, each cell is a different type of cell and/or have varying morphologies. In some embodiments, cells are imaged (1206) automatically to detect different parameters including density, mass, refractive index, morphology, size and texture of cell components in order to identify individual cells and their respective morphologies at the known locations within the nano-well array grid. Thus, allowing identified cells of interest to be recovered. In some embodiments, the method of automatically analyzing different cells utilizes staining the surface of the cell. In other embodiments, the method of automatically analyzing different cells utilizes internally staining the cell.

### E. Terminal Assay Using Reference Live Cell Array

In certain aspects, disclosed herein is a method for performing a terminal assay on a live cell array. In certain embodiments, the method comprises a) providing a population of target cells and an array of nano-wells; b) loading individual target cells of the target cells into individual wells of the array of nano-wells; c) growing a single-well colony of target cells from a target cell of a well; and d) performing a terminal assay on the individual cells.

In certain embodiments, a capture substrate is sealed onto the array of nano-wells, wherein each well is sealed by the capture substrate, wherein some cells of the single-well colony are attached to the capture substrate at locations on the capture substrate in which the locations are registered to the well position in the array of nano-wells. In certain embodiments, the capture substrate is separated from the array of nano-wells and measurements are performed on the some cells that are attached to the capture substrate at the locations. In certain embodiments, the measurements are performed on the individual cells in the individual wells, prior to colony growth. In certain embodiments, the measurements are performed on the single-well colony of target cells.

In certain embodiments, the measurements comprise image cytometry, secretion assay, or other measurements disclosed herein. In certain embodiments, the measurements are used to determine identity of the individual cells.

In certain embodiments, living cells within the single-well colony of target cells or clones are recovered based from the array of nano-wells or the capture substrate or a combination thereof.

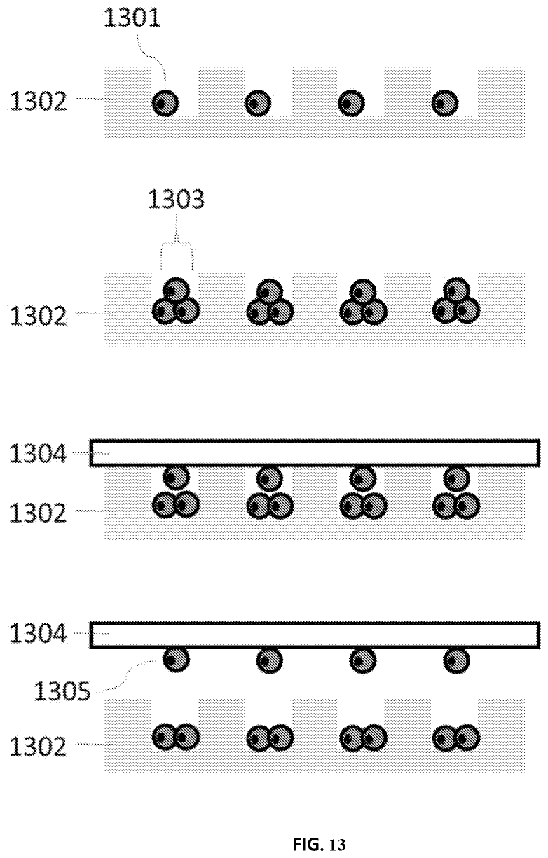

FIG. 13 shows a method for replicating individual cells in the array of nano-wells. Individual cells (1301) are loaded into individual wells of the plate (1302). Imaging and analysis are performed. The cells grow into a small-size colony of cells (1303). The nano-well plate is sealed substrate (1304). Some cells (1305) attach to the capture substrate. The capture substrate is separated from the nano-well plate. In some embodiments, an assay is performed on the substrate or on the cells in the well array. In other embodiments, a terminal assay as described herein is performed on the substrate. Cells are identified. Target cells are recovered from the nano-well plate or substrate for cloning. In some aspects, the target is a live cell.

### F. Antibody Discovery and Development

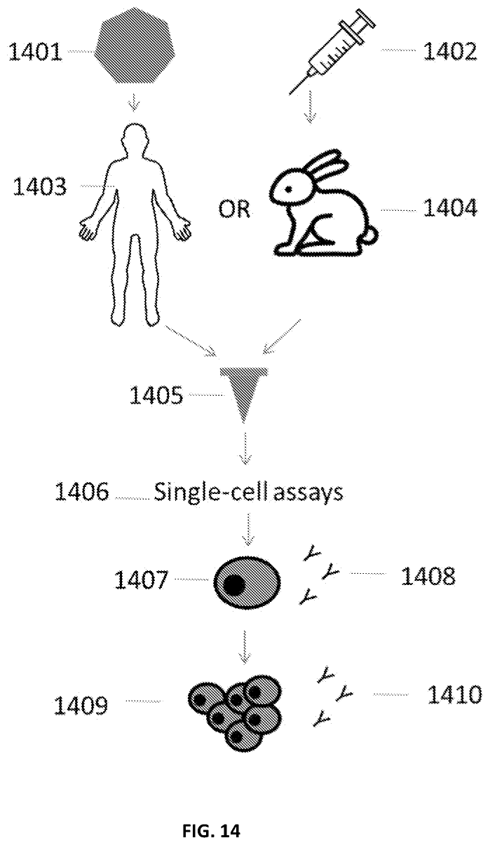

In certain embodiments, as described herein, is a method of antibody development and discovery, as seen in FIG. 14. A subject (1403 or 1404) is exposed to a pathogenic agent (1401) or a target antigen (1402). B cells and or other antibody producing cells (1405) are collected from the subject and analyzed using single-cell arrays (1406). Single cells of interest (1407) are isolated. The single cells may secrete biomolecules of interest (1408) such as antibodies. The single cells are amplified or the genetic sequence of the biomolecules of interest (1408) are recombinant expressed in a cell line (1409) and used to produce molecules of interest (1410)

In certain aspects, described herein is a method for high throughput identification of a B cell or other antibody secreting cells. In certain aspects, the method comprises obtaining a plurality of B cells or ACSs from a subject; loading the individual B cells or ASCs into individual wells of an array of nano-wells; growing the individual B cells or ASCs; detecting a property of the individual B cell or ASCs; and selecting the individual B cell or ASCs. In certain embodiments, the subject is a human. In certain embodiments, the subject is a non-human animal.

In certain embodiments, the subject has been immunized naturally through infection with a pathogenic agent. In certain embodiments, the pathogenic agent is a virus described herein. In certain embodiments, the subject has antibodies against cancer. In certain embodiments, the subject has antibodies against an autoimmune disease. In certain embodiments, the subject has antibodies against neurological disease. In certain embodiments, the subject has antibodies against metabolic, cardiovascular, endocrine disease. In certain embodiments, the subject has antibodies immune privileged tissues and cells in e.g. central nervous system, brain, eye, testes, gametes.

In certain embodiments, the subject has been immunized with a target antigen. The target antigen may be delivered in a variety of methods, such as injection and inhalation.

In certain embodiments, the subject has been immunized with adjuvant in combination or independent of the target antigen.

In some embodiments, B cells or ASCs are recovered from the subject. In certain embodiments, B cells or ASCs are recovered from blood samples. In certain embodiments, B cells or ASCs are recovered from the spleen, bone marrow, lymphatic system, or a combination thereof. The isolated B cells or ASCs are placed in proximity with an array of nano-wells described herein.

In certain embodiments, a property of the B cell or ASC is detected. The property may be detected using the methods described herein. In certain embodiments, the parameter comprises the immunophenotype of the cell of interest; the isotype immunoglobulin (Ig) subtype of the cell of interest or a secreted biomolecule; the affinity of the cell of interest or a secreted biomolecule; or the antigen specificity of the cell of interest or a secreted biomolecule. The immune phenotype may comprise CD19, CD20, CD38, or CD138. The Ig subtype may be IgG, IgM, IgE, IgD or IgA.

In certain embodiments, the B cell or ASCs may secrete a biomolecule of interest. In some embodiments, the biomolecule of interest is an antibody. In some embodiments, the antibody is screened for affinity and antigen specificity. In certain aspects, the antibody is an anti-cancer antibody. In some embodiments the antibody is an anti-autoimmune antibody, or a neurological disease antibody.

In certain embodiments, the B cell or ASC is be collected at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after immunization. In some embodiments, the B cell or ASC is collected at least about 1, 2, 3, 4, 5, 6, 7, 8, 9, 10, 11, or 12 months after immunization. In certain embodiments, the B cell or ASC is collected no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after immunization. In some embodiments, the B cell or ASC is collected no more than about 1, 2, 3, 4, 5, 6, 7, 8, 9, 10, 11, or 12 months after immunization.

In certain embodiments, the B cell or ASC is an antigen presenting cell. In certain embodiments, the antigen presenting cell is involved in the CD40 pathway. In certain embodiments, the B cell or ASC is involved in immune regulation and presentation. In certain embodiments, the B cell expresses ligands. In certain embodiments, the ligands interact with T cells or dendritic cell. In certain embodiments, the ligands comprise CD28, CD80, or CD86. In certain embodiments, the cells express cytokines. In certain embodiments, the cytokines regulate T cells, TH1/TH17/myeloid cells, neutrophils, macrophages, dendritic cells, natural Killer Cells, T regulatory cells, or CD4 T cells. In certain embodiments, the cytokines are selected from the list consisting of IL12, TH1, IL6, TH17, IL15, CD8, TL3, IL10, TGFβ, GM-CSF, and combinations thereof.

In certain embodiments, the B cell or ASC is for use in a method of cell therapy. In certain embodiments, the B cell or ASC is for use in production of a biomolecule of interest. In certain embodiments, the biomolecule of interest is an antibody. In certain embodiments, the B cell or ASC is amplified before recovery. In certain embodiments, the B cell or ASC is amplified after recovery. In certain embodiments, the time to reach a decision for selecting the target cell for recovery does not exceed 3 hours from the initialization of the method. In certain embodiments, the method yields clones with a mean productivity of 5 grams per liter.

In certain embodiments, the B cell or ASC is analyzed after recovery. In certain embodiments, the analysis comprises RT-PCR. In certain embodiments, the analysis identifies the sequence of a naturally paired antibody heavy chain gene, an antibody light chain gene, or both. In certain embodiments, the antibody is optimized. In certain embodiments, the optimization comprises altering a characteristic of the antibody to create a more suitable therapeutic. In certain embodiments, the optimized variable heavy chain and variable light chain of the antibody is expressed.

### G. Live Single-Cell Metabolic Assay

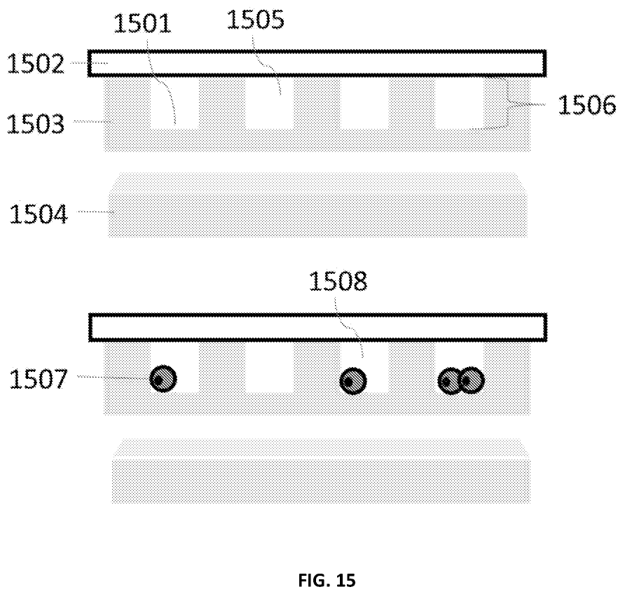

In certain instances, as described herein, and as seen in FIG. 15, a spectroscopic reference is acquired of amino acids in culture media (1505) in present in the nano-wells (1501) by a UV-Vis spectroscopic, optical absorption, optical transmission, or any combination thereof (1504). In some embodiments, where the amino acid media (1505) is sealed in sub-nanoliter wells (1501) of a sub-nanoliter well array (1503) by a capture substrate (1502). The pathlength (1506) is defined as the depth of the wells. Following acquisition of the reference spectrum, seed cells (1507) are loaded into the sub-nanoliter wells (1501) and the wells are re-sealed by the capture substrate (1502). Spectroscopic data is acquired of the live cells after every minute for a total of about 0 minutes to about 200 minutes. Spectroscopic data is acquired of the live cells after every minute for a total of about 0 minutes to about 1 minute, about 0 minutes to about 10 minutes, about 0 minutes to about 50 minutes, about 0 minutes to about 120 minutes, about 0 minutes to about 200 minutes, about 1 minute to about 10 minutes, about 1 minute to about 50 minutes, about 1 minute to about 120 minutes, about 1 minute to about 200 minutes, about 10 minutes to about 50 minutes, about 10 minutes to about 120 minutes, about 10 minutes to about 200 minutes, about 50 minutes to about 120 minutes, about 50 minutes to about 200 minutes, or about 120 minutes to about 200 minutes. Spectroscopic data is acquired of the live cells after every minute for a total of about 0 minutes, about 1 minute, about 10 minutes, about 50 minutes, about 120 minutes, or about 200 minutes. Spectroscopic data is acquired of the live cells after every minute for a total of at least about 0 minutes, about 1 minute, about 10 minutes, about 50 minutes, or about 120 minutes. Spectroscopic data is acquired of the live cells after every minute for a total of at most about 1 minute, about 10 minutes, about 50 minutes, about 120 minutes, or about 200 minutes. In some embodiments, individual cells showing modified function (1508) are recovered. In some embodiments, the effect of the amino acids in the media on the cells is then correlated to cell function.

### H. Cell Loading

In certain aspects, disclosed herein is a method comprising loading a plurality of target cells into an array of nano-wells.

In certain embodiments, the number of target cells per array of nano-wells is less than or equal to 10 to 1,000,000. the number of target cells per array of nano-wells is less than or equal to 5,000. In certain embodiments, the number of target cells per array of nano-wells is less than or equal to 10 to 30, 10 to 100, 10 to 300, 10 to 1,000, 10 to 10,000, 10 to 30,000, 10 to 100,000, 10 to 300,000, 10 to 1,000,000, 30 to 100, 30 to 300, 30 to 1,000, 30 to 10,000, 30 to 30,000, 30 to 100,000, 30 to 300,000, 30 to 1,000,000, 100 to 300, 100 to 1,000, 100 to 10,000, 100 to 30,000, 100 to 100,000, 100 to 300,000, 100 to 1,000,000, 300 to 1,000, 300 to 10,000, 300 to 30,000, 300 to 100,000, 300 to 300,000, 300 to 1,000,000, 1,000 to 10,000, 1,000 to 30,000, 1,000 to 100,000, 1,000 to 300,000, 1,000 to 1,000,000, 10,000 to 30,000, 10,000 to 100,000, 10,000 to 300,000, 10,000 to 1,000,000, 30,000 to 100,000, 30,000 to 300,000, 30,000 to 1,000,000, 100,000 to 300,000, 100,000 to 1,000,000, or 300,000 to 1,000,000. In certain embodiments, the number of target cells per array of nano-wells is less than or equal to 10, 30, 100, 300, 1,000, 10,000, 30,000, 100,000, 300,000, or 1,000,000. In certain embodiments, the number of target cells per array of nano-wells is less than or equal to at least 10, 30, 100, 300, 1,000, 10,000, 30,000, 100,000, or 300,000. In certain embodiments, the number of target cells per array of nano-wells is less than or equal to at most 30, 100, 300, 1,000, 10,000, 30,000, 100,000, 300,000, or 1,000,000.

In certain embodiments, the concentration of target cells on a per milliliter basis, of a sample, does not exceed 500 to 200,000. In certain embodiments, the concentration of target cells on a per milliliter basis, of a sample, does not exceed 500 to 1,000, 500 to 20,000, 500 to 100,000, 500 to 200,000, 1,000 to 20,000, 1,000 to 100,000, 1,000 to 200,000, 20,000 to 100,000, 20,000 to 200,000, or 100,000 to 200,000. In certain embodiments, the concentration of target cells on a per milliliter basis, of a sample, does not exceed 500, 1,000, 20,000, 100,000, or 200,000. In certain embodiments, the concentration of target cells on a per milliliter basis, of a sample, does not exceed at least 500, 1,000, 20,000, or 100,000. In certain embodiments, the concentration of target cells on a per milliliter basis, of a sample, does not exceed at most 1,000, 20,000, 100,000, or 200,000.

In certain embodiments, the sample volume containing target cells does not exceed 0.1 milliliters to 1.1 milliliters. In certain embodiments, the sample volume containing target cells does not exceed 0.1 milliliters to 0.3 milliliters, 0.1 milliliters to 0.5 milliliters, 0.1 milliliters to 0.7 milliliters, 0.1 milliliters to 0.9 milliliters, 0.1 milliliters to 1.1 milliliters, 0.3 milliliters to 0.5 milliliters, 0.3 milliliters to 0.7 milliliters, 0.3 milliliters to 0.9 milliliters, 0.3 milliliters to 1.1 milliliters, 0.5 milliliters to 0.7 milliliters, 0.5 milliliters to 0.9 milliliters, 0.5 milliliters to 1.1 milliliters, 0.7 milliliters to 0.9 milliliters, 0.7 milliliters to 1.1 milliliters, or 0.9 milliliters to 1.1 milliliters. In certain embodiments, the sample volume containing target cells does not exceed 0.1 milliliters, 0.3 milliliters, 0.5 milliliters, 0.7 milliliters, 0.9 milliliters, or 1.1 milliliters. In certain embodiments, the sample volume containing target cells does not exceed at least 0.1 milliliters, 0.3 milliliters, 0.5 milliliters, 0.7 milliliters, or 0.9 milliliters. In certain embodiments, the sample volume containing target cells does not exceed at most 0.3 milliliters, 0.5 milliliters, 0.7 milliliters, 0.9 milliliters, or 1.1 milliliters.

In certain embodiments, the single-cell loading efficiency of cells is based on parameters comprising concentration of cells, volume and time of cells placed in proximity of the array of nano-wells. In certain embodiments, the time for loading the individual target cells into the array of nano-wells and the secretion assay of the individual cells does not exceed 11 minutes. In certain embodiments, the time for loading the individual target cells into the array of nano-wells and secretion assay of the individual target cells does not exceed 6 minutes. In certain embodiments, the time for loading the individual target cells into the array of nano-wells and secretion assay of the individual target cells does not exceed 2 minutes. In certain embodiments, the time for loading the individual target cells into the array of nano-wells and secretion assay of the individual target cells does not exceed 1 minute.

In some embodiments, the single-cell loading efficiency is equal to the number of nano-wells occupied by cells after loading versus the total number of nano-wells of the array of nano-wells. In some embodiments, the single-cell loading efficiency of cells is 0 percent to 100 percent. In some embodiments, the single-cell loading efficiency of cells is 0 percent to 1 percent, 0 percent to 15 percent, 0 percent to 33 percent, 0 percent to 55 percent, 0 percent to 100 percent, 1 percent to 15 percent, 1 percent to 33 percent, 1 percent to 55 percent, 1 percent to 100 percent, 15 percent to 33 percent, 15 percent to 55 percent, 15 percent to 100 percent, 33 percent to 55 percent, 33 percent to 100 percent, or 55 percent to 100 percent. In some embodiments, the single-cell loading efficiency of cells is 0 percent, 1 percent, 15 percent, 33 percent, 55 percent, or 100 percent. In some embodiments, the single-cell loading efficiency of cells is at least 0 percent, 1 percent, 15 percent, 33 percent, or 55 percent. In some embodiments, the single-cell loading efficiency of cells is at most 1 percent, 15 percent, 33 percent, 55 percent, or 100 percent.

In some embodiments, an array of nano-wells is loaded 0 times to 20 times. In some embodiments, an array of nano-wells is loaded 0 times to 1 time, 0 times to 2 times, 0 times to 3 times, 0 times to 5 times, 0 times to 10 times, 0 times to 20 times, 1 time to 2 times, 1 time to 3 times, 1 time to 5 times, 1 time to 10 times, 1 time to 20 times, 2 times to 3 times, 2 times to 5 times, 2 times to 10 times, 2 times to 20 times, 3 times to 5 times, 3 times to 10 times, 3 times to 20 times, 5 times to 10 times, 5 times to 20 times, or 10 times to 20 times. In some embodiments, an array of nano-wells is loaded 0 times, 1 time, 2 times, 3 times, 5 times, 10 times, or 20 times. In some embodiments, an array of nano-wells is loaded at least 0 times, 1 time, 2 times, 3 times, 5 times, or 10 times. In some embodiments, an array of nano-wells is loaded at most 1 time, 2 times, 3 times, 5 times, 10 times, or 20 times.

### I. Selection

In some embodiments, the time to reach a decision for selecting a target cell, from initialization of the method, does not exceed 0.5 hours to 6 hours. In some embodiments, the time to reach a decision for selecting a target cell, from initialization of the method, does not exceed 0.5 hours to 1 hour, 0.5 hours to 2 hours, 0.5 hours to 2 hours, 0.5 hours to 4 hours, 0.5 hours to 5 hours, 0.5 hours to 6 hours, 1 hour to 2 hours, 1 hour to 3 hours, 1 hour to 4 hours, 1 hour to 5 hours, 1 hour to 6 hours, 2 hours to 4 hours, 2 hours to 5 hours, 2 hours to 6 hours, 4 hours to 5 hours, 4 hours to 6 hours, or 5 hours to 6 hours. In some embodiments, the time to reach a decision for selecting a target cell, from initialization of the method, does not exceed 0.5 hours, 1 hour, 2 hours, 3 hours, 4 hours, 5 hours, or 6 hours. In some embodiments, the time to reach a decision for selecting a target cell, from initialization of the method, does not exceed at least 0.5 hours, 1 hour, 2 hours, 3 hours, 4 hours, or 5 hours. In some embodiments, the time to reach a decision for selecting a target cell, from initialization of the method, does not exceed at most 1 hour, 2 hours, 3 hours, 4 hours, 5 hours, or 6 hours.

In certain embodiments, the time to reach a decision for selecting a target cell does not exceed 1 cell doubling time to 10 cell doubling times. In certain embodiments, the time to reach a decision for selecting a target cell does not exceed 1 cell doubling time to 3 cell doubling times, 1 cell doubling time to 5 cell doubling times, 1 cell doubling time to 10 cell doubling times, 3 cell doubling times to 5 cell doubling times, 3 cell doubling times to 10 cell doubling times, or 5 cell doubling times to 10 cell doubling times. In certain embodiments, the time to reach a decision for selecting a target cell does not exceed 1 cell doubling time, 3 cell doubling times, 5 cell doubling times, or 10 cell doubling times. In certain embodiments, the time to reach a decision for selecting a target cell does not exceed at least 1 cell doubling time, 3 cell doubling times, or 5 cell doubling times. In certain embodiments, the time to reach a decision for selecting a target cell does not exceed at most 3 cell doubling times, 5 cell doubling times, or 10 cell doubling times.

In certain embodiments, the method yields clones with a mean productivity within a range of a 5 to 12 grams per liter. In certain embodiments, the method yields clones with a mean productivity within a range of 1 to 5 grams per liter. In certain instances, the method yields clones with a productivity within a range of 0.1 to 1 gram per liter. In certain embodiments, the method yields clones with a mean productivity of 1 gram per liter to 14 grams per liter. In certain embodiments, the method yields clones with a mean productivity of 1 gram per liter to 5 grams per liter, 1 gram per liter to 10 grams per liter, 1 gram per liter to 12 grams per liter, 1 gram per liter to 14 grams per liter, 5 grams per liter to 10 grams per liter, 5 grams per liter to 12 grams per liter, 5 grams per liter to 14 grams per liter, 10 grams per liter to 12 grams per liter, 10 grams per liter to 14 grams per liter, or 12 grams per liter to 14 grams per liter. In certain embodiments, the method yields clones with a mean productivity of 1 gram per liter, 5 grams per liter, 10 grams per liter, 12 grams per liter, or 14 grams per liter. In certain embodiments, the method yields clones with a mean productivity of at least 1 gram per liter, 5 grams per liter, 10 grams per liter, or 12 grams per liter. In certain embodiments, the method yields clones with a mean productivity of at most 5 grams per liter, 10 grams per liter, 12 grams per liter, or 14 grams per liter.

In certain embodiments, a collection of proof images is acquired of individual nano-wells of the array of nano-wells during events comprising loading of the target cell into the array of nano-wells, the secretion titration measurement, or recovery of the single target cell, or a combination thereof. In some embodiments, a collection of proof images is acquired at each step during the method. In certain embodiments, data from the measurements of individual target cells is used to create a training set to predict cellular function.

### J. Recovery

In certain embodiments, once cells of interest have been selected, they are recovered. Micromanipulator pipetting methods are used in these instances. In certain embodiments, the cells that are recovered are live cells.

### K. Capture Methods

In certain embodiments, a substrate is provided, further wherein one or more capture reagents for the article is immobilized to the capture substrate. In certain embodiments, the capture substrate is placed in proximity of the array of nano-wells before, during or after exposure of the one or more reagents to the target cells. In certain embodiments, measurements of the articles are obtained on a surface of the substrate. In certain embodiments, the measurements of the articles obtained on the surface of the substrate comprise bright field microscopy, fluorescence microscopy, microscopy utilizing a laser source and photomultiplier tube detector, or a combination thereof.

In certain embodiments, the article is captured on the capture substrate. In certain embodiments, the capture substrate is comprised of a hard material. In certain embodiments, the capture substrate is comprised of a soft material. In certain embodiments, the hard material comprises a transparent plastic or a transparent glass material. In certain embodiments, the soft material comprises a transparent elastomeric material. In some embodiments, a reflective material is coated on the capture substrate.

In certain embodiments, the article is captured on a plurality of beads inside of the well. In certain embodiments, the article is captured on an interior surface of the well. In certain embodiments, the article is captured within a matrix contained within the well.

In certain embodiments, the time for capturing biomolecules on the capture substrate after sealing the array of nano-wells does not exceed 29 minutes. In certain embodiments, the time for capturing biomolecules on the capture substrate surface after sealing the array of nano-wells does not exceed 11 minutes. In certain embodiments, the time for capturing biomolecules on the capture substrate after sealing the array of nano-wells does not exceed 4 minutes. In certain embodiments the time for capturing biomolecules on the capture substrate after sealing the array of nano-wells does not exceed 2 hours.

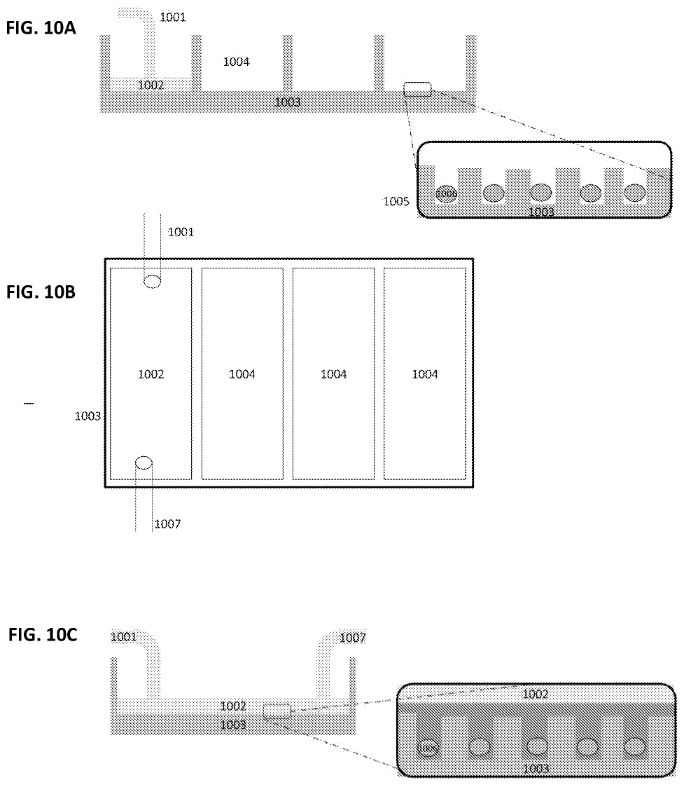

Described herein are methods of capturing biomolecules secreted by the target cell as shown in FIG. 9. In some embodiments as seen in FIG. 9A, the target cell (901) is located in the nano-well (902). In some embodiments, the secreted biomolecule (903) is captured by the capture substrate (904). In some embodiments, as shown in FIG. 9B, the target cell (901) is located within the nano-well (902), wherein beads (905) functionalized with a capture agent, capture the secretion product within the well. In some embodiments, as shown in FIG. 9C, the target cell (901) is located within the nano-well (902), wherein capture agents are attached to the interior surface of the well to capture secreted product there. In some embodiments, as shown in FIG. 9D, the nano-well is filled with a hydrogel or other semi solid medium (906). In these embodiments, the capture agent is located within the hydrogel or part of the hydrogel.

### L. Biosimilar Development and Clonal Selection Based on Key Product Attributes of Glycosylation and Aggregation

Described herein are certain embodiments of methods for biosimilar development and clonal selection based on key product attributes of glycosylation and aggregation. In some embodiments, the purpose of methods is to identify desired or undesired changes in glycosylation patters on antibodies or more generally, biomolecules, at the single-cell stage to aid in cell selection for optimal target biomolecule product quality attributes.

Biosimilar development and clonal selection based on key product attributes as follows: —comprise glycosylation, aggregation, modifications of amino acids, N terminal heterogeneity, C-terminal heterogeneity or disulfide bonds. In some embodiments, glycosylation comprises sialylation, fucosylation, galactosylation or branching. In some embodiments, aggregation assays are utilized to identify changes in secreted product and or select cells. In some embodiments modifications of amino acids comprising Deamidation, Isomerization, Glycation, or Oxidation are analyzed for cell selection. In some embodiments, N terminal heterogeneity comprising formation of Pyroglutamate is analyzed for cell selection. In some embodiments C terminal heterogeneity comprising lysine variants or amidation is analyzed for cell selection. In some embodiments, disulfide bonds, free thiols, or thioethers are analyzed for cell selection. In some embodiments, disulfide shuffling is analyzed for cell selection. In some embodiments, fragmentation of cleavage in the hinge region of Asp-Pro is analyzed for cell selection.

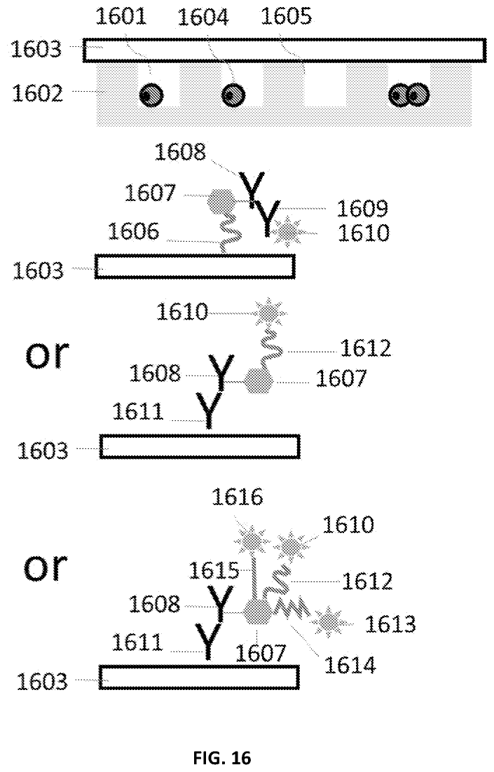

In some embodiments, methods for biosimilar development and clonal selection based on key product attributes of glycosylation utilize analysis by lectin affinity binding, as shown in FIG. 16. Live single-cell separation is shown in FIG. 16A where cells (1604) are shown in individual nano-wells (1605) of an array of nano-wells (1602). In some embodiments, the cells (1604) are sealed in the nano-wells (1605) along with reagents (1601) by a substrate lid (1603). In some embodiments, analytics comprising image cytometry or a secretion assay, are performed on the cells. In some embodiments, analytics are performed on the cells (1604) in the individual nano-wells (1605). In some embodiments, the cells are live. In some embodiments, analytics are performed on products secreted onto the substrate lid (1603). In some embodiments, the substrate lid (1605) is also referred to as the capture substrate. In some embodiments a single glycan assay is performed. In some embodiments, a multiplexed glycan assay is performed by either multi detection per substrate and or multiple rounds of secretion assays with multiple substrates. In some embodiments, capture of the secreted antibody or biomolecule on one or multiple (sequential prints) capture substrate utilizes a panel of binding molecules comprising lectins or other glycan binding molecules. In some embodiments, 1 to about 20 binding molecules comprise the panel. In some embodiments, anti IG and a secondary detection anti glycan lectin panel are utilized. In some embodiments, binding is compared to a reference glycan binding profile.

In some embodiments, one or more antiglycan lectin or other capture reagents (1606) are immobilized to the substrate lid (1603) as shown in FIG. 16B. In some embodiments, capture reagent is an antibody or an antibody mimetic as described herein, wherein the antibody or antibody mimetic binds to specific glycans. In some embodiments, the lectin electively binds core-fucose, sialic acids, N-acetyl-D-lactosamine (Galβ1-4GlcNAc), mannose, N-acetylglucosamine (GlcNAc), or other specific molecules. In some embodiments, glycan (1607) is captured by the capture reagent (1606). In some embodiments, detection reagents comprising an antibody or other biomolecule (1608), an anti-biomolecule antibody (1609)labeled with a particular fluorophore (1610) as shown in FIG. 16B are used for detection of glycan. In some embodiments, as shown in FIG. 16C, an anti-biomolecule is the capture reagent (1611) which captures an antibody or biomolecule (1608), in turn capturing glycan (1607). In some embodiments, detection reagents comprising anti-glycan lectin or other moieties (1612) labeled with a particular fluorophore (1610), are used to detect the presence of glycan (1607). In some embodiments, as shown in FIG. 16D, an anti-biomolecule is the capture reagent (1611) which captures an antibody or biomolecule (1608), in turn capturing glycan (1607). In some embodiments, the glycan (1607) captures multiple detection reagents. In some embodiments a detection reagent of the multiple detection reagents is anti-glycan lectin or other detection reagent (1615) labeled with a first fluorophore (1616). In some embodiments a detection reagent of the multiple detection reagents is anti-glycan lectin or other moieties (1612) labeled with a second fluorophore (1610), wherein the second fluorophore (1610) is detectable distinct from the first fluorophore (1616). In some embodiments a detection reagent of the multiple detection reagents is anti-glycan lectin or other moieties (1614) labeled with a third fluorophore (1613), wherein the third fluorophore (1613) is detectable distinct from both the first fluorophore (1616) and the second fluorophore (1610).

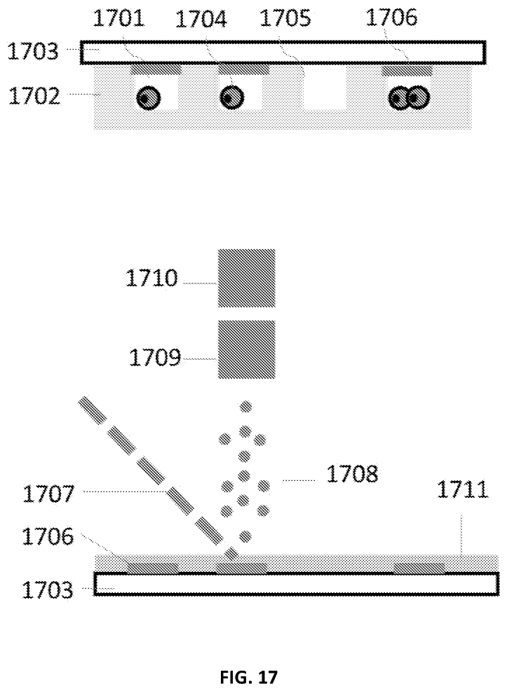

Described herein are methods for biosimilar development and clonal selection based on key product attributes of glycosylation utilizing mass spectrometry. Live single-cell separation is shown FIG. 17A where cells (1704) are shown in individual nano-wells (1705) of an array of nano-wells (1702). In some embodiments, the cells (1704) are sealed in the nano-wells (1705) along with reagents (1701) by a substrate lid (1703). In some embodiments, analytics comprising image cytometry or a secretion assay, are performed on the cells. In some embodiments, analytics are performed on the cells (1704) in the individual nano-wells (1705). In some embodiments, analytics are performed on products (1706) secreted onto the substrate lid (1703). In some embodiments, the substrate lid (1705) is also referred to as the capture substrate. After capture of cell secreted products (1706), the substrate lid (1703) is removed and coated with a matrix (1711) for mass spectrometry, as shown in FIG. 17B. In some embodiments, mass spectrometry comprises matrix assisted laser desorption and ionization time of flight (MALDI-TOF) mass spectrometry. In some embodiments, mass spectrometry comprises mass spectrometry imaging (MSI).

In some embodiments, an energy beam is directed to the surface, causing the secreted products (1706) to be desorbed and ionized. The desorbed and ionized secreted products (1708) are then detected (1710) and analyzed (1709) to identify the secreted product utilizing a mass spectrometry reference database. In some embodiments, multiple substrate lids (1703) or capture substrates are used on a single array of nano-wells (1702) loaded with cells (1704) to analyze secretion profiles over time utilizing mass spectrometry analysis. Described herein are methods that for biosimilar development and clonal selection based on key product attribute of glycosylation that in some embodiments, utilize aggregation assays. In some embodiments, the purpose of this analysis is to identify undesired antibody glycosylation or aggregation or more generally, biomolecule glycosylation or aggregation at the single-cell stage.

In some embodiments, single cells are isolated in individual wells of an array of nano-wells, wherein the single cells produce antibodies or other biomolecules with or without Fc domains. In some embodiments, the produced or secreted biomolecules are captured on the capture substrate. Next, in some embodiments, the secreted biomolecules are interrogated with a stain that indicates aggregation. In some embodiments, aggregation data is used to inform which single-cell to clone for production of antibodies or other biomolecules with or without an Fc domain.

In some embodiments, clones are selected with differentiated Glycan profile. We are selecting clones based on glycan profiles. Antibodies with undesired glycans have undesired pharmacological functions and are therefore not selected.

In some embodiments, the method yields clones with less than 1 percent aggregation to 11 percent aggregation. In some embodiments, the method yields clones with less than 1 percent aggregation to 3 percent aggregation, 1 percent aggregation to 7 percent aggregation, 1 percent aggregation to 9 percent aggregation, 1 percent aggregation to 11 percent aggregation, 3 percent aggregation to 7 percent aggregation, 3 percent aggregation to 9 percent aggregation, 3 percent aggregation to 11 percent aggregation, 7 percent aggregation to 9 percent aggregation, 7 percent aggregation to 11 percent aggregation, or 9 percent aggregation to 11 percent aggregation. In some embodiments, the method yields clones with less than 1 percent aggregation, 3 percent aggregation, 7 percent aggregation, 9 percent aggregation, or 11 percent aggregation. In some embodiments, the method yields clones with less than at least 1 percent aggregation, 3 percent aggregation, 7 percent aggregation, or 9 percent aggregation. In some embodiments, the method yields clones with less than at most 3 percent aggregation, 7 percent aggregation, 9 percent aggregation, or 11 percent aggregation.

### M. Quantitative Single-Cell Secretion Measurements

Described herein are certain embodiments of quantitative measurement of signals indicating biomolecules secreted by individual cells in individual nano-wells of the array of nano-wells. In some embodiments, the quantification involves normalizing measurements of single-cell secretions of biomolecules to one or more controls. In some embodiments, the controls comprise a reference reagent, a background reagent, or a combination thereof.

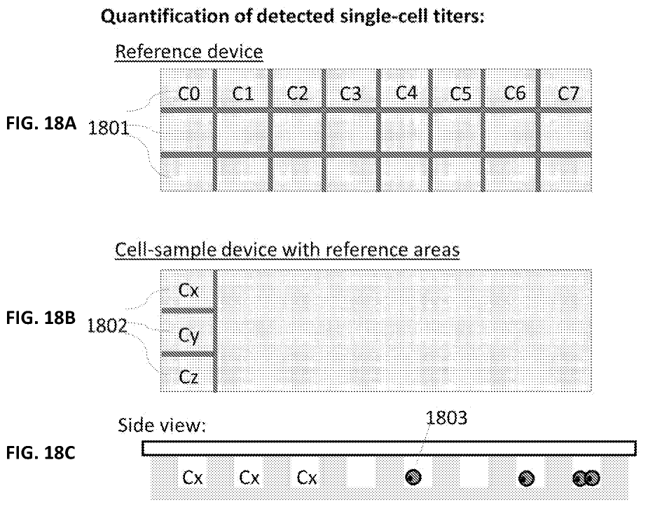

In some embodiments, a reference reagent is of known composition, concentration and of a known location on an array of nano-wells or capture substrate. In some embodiments, the known location of the reference reagent is referred to as a reference area. In some embodiments, reference areas are physically separate areas that are located on a reference device (1801) FIG. 18A. In some embodiments, reference areas (1802) are located along side cells (1803) loaded into individual wells of an array of nano-wells as seen in FIGS. 18B-18C (showing a top view and side view, respectively). In some embodiments, reference areas are also located on the corresponding capture substrate. In some embodiments, reference reagents comprise labels. In some embodiments, labels are fluorophores. In some embodiments, the label is attached to a secondary antibody that binds specifically to the reference reagents. In some embodiments, the reference reagent is bound to a capture reagent attached to a substrate, within a reference area, on the capture substrate.

Described herein are certain embodiments for the manufacture of reference areas. In some embodiments, reference areas are separated from other reference areas by hydrophobic barriers, trenches or walls. In some embodiments, hydrophobic barriers are fabricated by a hydrophobic pen. In some embodiments, the trenches surrounding reference areas are created by subtractive techniques such as laser scribing or photolithographic masking followed by dry or wetting etching. In some embodiments, the reference areas are separated by walls made by additive processes such as 3D printing or photolithography where the developed resist material is left in place to form the walls.

In some embodiments, label-free detection is used to measure the reference reagent signal. In some embodiments, fluorescence detection is used to measure the reference reagent signal. In some embodiments, the recorded data is stored along with the settings of the system. In some instances, the system may comprise a microarray scanner, a microscope, a PMT, a laser excitation source or an LED excitation source, a camera or a combination thereof.

In some embodiments, the method described herein is performed using a reference device, or reference array, and performed in parallel with each experimental device, or experimental array. In some embodiments, reference reagent signals are detected from the reference areas of a reference device, as seen in FIG. 18A, before each experiment.

In some embodiments, a dilution series of a reference reagent, comprising recombinant human Ig solution, or human Ig supernatant from cell culture in culture media, both of known initial concentrations, is prepared and applied to the reference area. In some embodiments, a reference reagent of a known dilution factor is repeatedly applied to multiple reference areas, wherein each of the areas is a replicate of the other in terms of dilution factor. In some embodiments, a series comprising reference reagent set at different dilution factors is distributed across a plurality of reference areas, as seen in FIG. 18A (1801). Reference reagent signals extracted from these reference areas comprise a reference standard curve for calibrating single cell secretion measurements to a known concentration, thus quantifying the single cell secretion measurement. The two axes of the reference standard curve are reference reagent signal versus concentration. In some embodiments, the reference reagent signal is measured directly from an array of nano-wells. In some embodiments, the reference reagent signal is measured from a capture substrate. In some embodiments, the reference reagent signal is measured from the separate reference device. In some embodiments, the reference areas of a capture substrate are microengraved, or equivalently stated, the capture substrate is sealed onto the cell containing array of nano-wells for 1 minute, 5 minutes, 10 minutes, 20 minutes, or greater than 20 minutes. In some embodiments, the concentration of molecules secreted by an individual cell of an individual nano-well of the array of nano-wells are interpolated from the reference standard curve, thus quantifying the signal. Further, in some embodiments, the number of molecules secreted per minute are then determined using numerical simulations by solving the partial differential equations relating the secretion, diffusion, and binding of analytes with a specific capture antibody

In some embodiments, each concentration on the standard curve is represented by one or more replicates.

### N. Quantitative Single-Cell Secretion Measurements, Utilizing a Background Control

Described herein are certain embodiments for quantitative measurements of single-cell secretion, utilizing a background control. In some embodiments, the purpose of the background control is to create an alignment grid correlating the single-cell secreted biomolecules, on the surface of the capture substrate, to the location of the nano-well containing the single cell that secreted the biomolecules. In some embodiments, the purpose of the background control is to normalize signal variation at the individual nano-well level. In some embodiments, the background control is used for normalizing signal variation due to uncontrolled experimental factors that affect all experimental channels. In some embodiments, a background control refers to an experimental control that comprises a background reagent that is detectably different than the single-cell secreted biomolecule and the reference reagent. In some embodiments the background reagent comprises a background reagent label that is detectably different than that the label used to detect the single-cell secreted biomolecule and the reference reagent. In some embodiments the background reagent is readout by a microscopy imaging channel that is separate from the channel used to detect the single-cell secreted biomolecule and the reference reagent. In some embodiments, the background channel is configured for label-free detection of the background reagent, where the label-free background reagent is detectably different from the single-cell secreted biomolecule within the background channel. In some embodiments, the background reagent is in each nano-well of the array of nano-wells. In these embodiments, the background reagent, background reagent label or a combination thereof is measured directly in the nano-well or the on the capture substrate after microengraving. In some embodiments, a background reagent is in a nano-well that does not contain a cell.

In some embodiments, the measurement of the single-cell secreted biomolecule, normalized by the reference reagent through the reference standard curve, is further normalized by the background control. In some embodiments, the reference standard curve is obtained from the reference areas of the reference device or from the device containing cells by co-loading a single concentration of background reagent with the reference reagent into individual wells of the array of nano-wells. In these embodiments, the single-cell secretion measurements are both quantified to a known concentration, using the reference standard curve and reduced in variation using the background control.

### O. Assay for Determining Binding Parameters

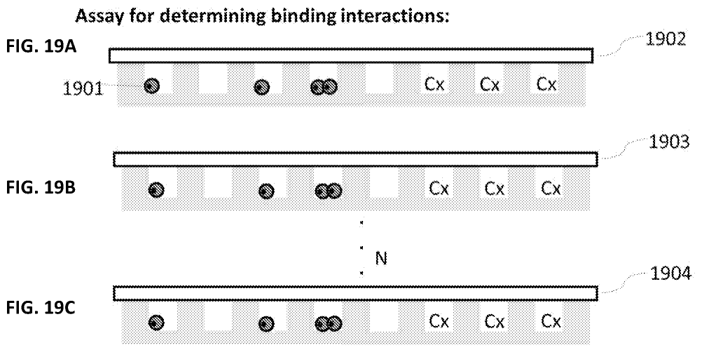

Described herein are certain assay methods for determining binding interaction parameters and enabling quantitative measurements. In some embodiments, individual cells (1901) are loaded into individual nano-wells of an array of nano-wells, and reference reagents of known concentration, within the linear range of the standard curve, are loaded in reference areas. In some embodiments of the assay method, time intervals for sealing the capture substrate to the array of nano-wells, or equivalently stated, microengraving time, is 2 min (1902), 4 min (1903), or 20 min (1904) as seen in FIGS. 19A, 19B and 19C, respectively. In some embodiments, the media in the wells is exchanged after each microengraving. In some embodiments, the capture substrate is scanned after separation from the array of nano-wells, to measure the amount of secretion at each location on the capture substrate corresponding to each nano-well. In some embodiments, data is collected continuously throughout the experiment and readout is label-free. In some embodiments, assay data obtained from individual cell secretion is compared against the background channel for scaling the signal. In some embodiments, the scaled data is quantified against the reference data to determine concentration of molecules secreted from individual cells. In some embodiments, the binding interactions and binding interaction parameters are determined for individual cell titers from a curve of signal versus microengraving time. Additionally, in some embodiments, capture substrates are washed in increasing time intervals and scanned after each wash to obtain signal. In these embodiments, a curve for de-binding (e.g. dissociation) of secreted analyte is created and used to determine parameters for de-binding of the cell-secreted biomolecules. In some embodiments, the assay for determining binding parameters can be in real-time. The methods described herein allow for quantitative measurement.

### P. A Method for Selecting a Target Cell

### Method Overview

Placing a Plurality of Cells into a Plurality of Nano-Wells

Described herein is a method of selecting a target cell, wherein the target cell may be a certain type of cell. In certain embodiments, the target cell is a T cell, a B cell, a plasma cell, antibody secreting cells (ASCs), an antigen presenting cell, a hybridoma, an immune cell, a stem cell, an induced pluripotent stem cell (IPSC), or an engineered cell. In certain embodiments, the engineered cell is a CHO cell, a HEK 293 cell, a murine NSO cell, CAP cell, AGE cell, SP2/0, BHK21, HKB-11, HuH-7, C127, TKT, HT-1080 cell, a HELA cell, engineered B cell, engineered NK cell, engineered T cell such as CAR T cell, engineered dendritic cell, an engineered antigen presenting cell, or differentiated IPSC. In certain embodiments, the cell is a lymphocyte, leukocytes tumor cell, stromal cell, neuronal cell, stem cell, gametes such as sperm cell and ova cell, or an embryo. In certain embodiments, the cell is a primary cell, a cell line, an eukaryotic cell, prokaryotic cell, a yeast cell, a bacterial cell, an E. coli cell or a P. pastoris cell.

In certain embodiments, the number of cells present on a plurality of nano-wells is 100 to 10,000,000. In certain embodiments, the number of cells present on a plurality of nano-wells is 100 to 1,000, 100 to 10,000, 100 to 100,000, 100 to 1,000,000, 100 to 10,000,000, 1,000 to 10,000, 1,000 to 100,000, 1,000 to 1,000,000, 1,000 to 10,000,000, 10,000 to 100,000, 10,000 to 1,000,000, 10,000 to 10,000,000, 100,000 to 1,000,000, 100,000 to 10,000,000, or 1,000,000 to 10,000,000. In certain embodiments, the number of cells present on a plurality of nano-wells is 100, 1,000, 10,000, 100,000, 1,000,000 or 10,000,000. In certain embodiments, the number of cells present on a plurality of nano-wells is at least 100, 1,000, 10,000, or 100,000. In certain embodiments, the number of cells present on an plurality of nano-wells is at most 1,000, 10,000, 100,000, 1,000,000 or 10,000,000.

Described herein is a method of selecting a target cell, wherein a plurality of nano-wells is utilized. In certain embodiments, the nano-wells are comprised of a hard material. In certain embodiments, the nano-wells are comprised of a soft material. In certain embodiments, the hard material comprises a transparent plastic or a transparent glass material, or a reflective material. In certain embodiments, the soft material comprises a transparent elastomeric material.

In some embodiments, the individual nano-well has a volume of 10 picoliters to 2,000 picoliters. In some embodiments, the well has a volume of 10 picoliters to 100 picoliters, 10 picoliters to 250 picoliters, 10 picoliters to 500 picoliters, 10 picoliters to 1,000 picoliters, 10 picoliters to 1,500 picoliters, 10 picoliters to 2,000 picoliters, 100 picoliters to 250 picoliters, 100 picoliters to 500 picoliters, 100 picoliters to 1,000 picoliters, 100 picoliters to 1,500 picoliters, 100 picoliters to 2,000 picoliters, 100 picoliters to 3000 picoliters, 100 picoliters to 5000 picoliters, 100 picoliters to 8000 picoliters, 100 picoliters to 10 nanoliters, 100 picoliters to 50 nanoliters, 100 picoliters to 100 nanoliters, 100 picoliters to 200 nanoliters, 100 picoliters to 300 nanoliters, 100 picoliters to 400 nanoliters, 100 picoliters to 500 nanoliters, 100 picoliters to 600 nanoliters, 100 picoliters to 700 nanoliters, 100 picoliters to 800 nanoliters, 100 picoliters to 900 nanoliters, 100 picoliters to 1000 nanoliters, 250 picoliters to 500 picoliters, 250 picoliters to 1,000 picoliters, 250 picoliters to 1,500 picoliters, 250 picoliters to 2,000 picoliters, 500 picoliters to 1,000 picoliters, 500 picoliters to 1,500 picoliters, 500 picoliters to 2,000 picoliters, 1,000 picoliters to 1,500 picoliters, 1,000 picoliters to 2,000 picoliters, or 1,500 picoliters to 2,000 picoliters. In some embodiments, the individual well has a volume of 10 picoliters, 100 picoliters, 250 picoliters, 500 picoliters, 1,000 picoliters, 1,500 picoliters, or 2,000 picoliters. In some embodiments, the well has a volume of at least 10 picoliters, 100 picoliters, 250 picoliters, 500 picoliters, 1,000 picoliters, or 1,500 picoliters. In some embodiments, the well has a volume of at most 100 picoliters, 250 picoliters, 500 picoliters, 1,000 picoliters, 1,500 picoliters, 2,000 picoliters, 10 nanoliters, 50 nanoliters, 100 nanoliters, 200 nanoliters, 300 nanoliters, 400 nanoliters, 500 nanoliters, 600 nanoliters, 700 nanoliters, 800 nanoliters, 900 nanoliters, or 1000 nanoliters.

In some embodiments, the plurality of cells and the plurality of nano-wells described herein comprise attributes as described in Section II.

The method described herein comprises placing a plurality of cells into an array of nano-wells. In certain embodiments, the number of cells per a well of the plurality of nano-wells is about 0 to about 50. In certain embodiments, the number of cells per a well of the plurality of nano-wells is about 0 to about 1, about 0 to about 5, about 0 to about 10, about 0 to about 50, about 1 to about 5, about 1 to about 10, about 1 to about 50, about 5 to about 10, about 5 to about 50, or about 10 to about 50. In certain embodiments, the number of cells per a well of the plurality of nano-wells is about 0, about 1, about 5, about 10, or about 50. In certain embodiments, the number of cells per a well of the plurality of nano-wells is at least about 0, about 1, about 5, or about 10. In certain embodiments, the number of cells per a well of the plurality of nano-wells is at most about 1, about 5, about 10, or about 50. In certain embodiments, the number of cells per a well of the plurality of nano-wells is 1.

In certain embodiments, the step of placing the plurality of cells into the plurality of nano-wells is done in no more than 20, 19, 18, 17, 16, 15, 14, 13, 12, 11 minutes. In other embodiments, the step of placing the plurality of cells into the plurality of nano-wells is done in no more than 10, 9, 8, 7, 6, 5, 4, 3, 2, or 1 minute(s). In other embodiments, the step of placing the plurality of cells into the plurality of nano-wells is done in about 20, 19, 18, 17, 16, 15, 14, 13, 12, 11 minutes. In other embodiments, the step of placing the plurality of cells into the plurality of nano-wells is done in about 10, 9, 8, 7, 6, 5, 4, 3, 2, or 1 minute(s).

Treating and/or Modifying Nano-Wells Etc.

In some embodiments, the method described herein comprises a step of exposing at least the subset of the plurality of nano-wells described herein to a condition, wherein the condition is treating the individual nano-well with one or more reagents, treating the individual nano-well with a plurality of secondary cells, applying a membrane to the individual nano-well to form an individual membrane-modified nano-well, contacting the individual nano-well with a capture substrate, or contacting the individual nano-well with a secondary cell-immobilized capture substrate, or a combination thereof. In some specific embodiments, the one or more reagents, the plurality of secondary cells, the capture substrate, the secondary cell-immobilized capture substrate described herein comprise attributes as described in Section II.

In some embodiments, the step described herein is performed while the plurality of cells receive reduced perturbations when compared with corresponding perturbations received by a comparative plurality of cells in a cell line development process of (i) limiting dilution selection, (ii) fluorescence-activated cell sorting (FACS), (iii) isolating individual cells with cloning cylinders, or (iv) flow cytometry. In some embodiments, the perturbation described herein is biological perturbation with regard to the cells or the solution/environment of the cells. In other embodiments, the perturbation described herein is chemical perturbation with regard to the cells or the solution/environment of the cells. In other embodiments, the perturbation described herein is mechanical perturbation with regard to the cells or the solution/environment of the cells.

In specific embodiments, the reduced perturbations described herein are more stable and/or more optimal pH value for the plurality of cells in the plurality of nano-wells during the described treatment or modification. In specific embodiments, the reduced perturbations described herein are more stable and/or more optimal osmolarity value for the plurality of cells in the plurality of nano-wells during the described treatment or modification. In specific embodiments, the reduced perturbations described herein are more stable and/or more optimal temperature for the plurality of cells in the plurality of nano-wells during the described treatment or modification. In specific embodiments, the reduced perturbations described herein are more stable and/or more optimal humidity for the plurality of cells in the plurality of nano-wells during the described treatment or modification. In specific embodiments, the reduced perturbations described herein are more stable and/or more optimal ingredients of cell culture media for the plurality of cells in the plurality of nano-wells during the described treatment or modification. In specific embodiments, the reduced perturbations described herein are less mechanical compression for the plurality of cells in the plurality of nano-wells during the described treatment or modification. In specific embodiments, the reduced perturbations described herein are less hydrostatic pressure for the plurality of cells in the plurality of nano-wells during the described treatment or modification. As a result, in some embodiments, the method described herein does not alter or change little the morphology of the plurality of cells.

In some embodiments, the method described herein does not alter or change little the migration of the plurality of cells. In some embodiments, the method described herein does not alter or change little the growth rate of the plurality of cells. In some embodiments, the method described herein does not alter or change little the expression of external-stress-sensing gene(s) in the plurality of cells.

### Detecting a Signal or a Change Thereof

In some embodiments, the methods described herein comprise detecting a signal or a change thereof from a particular nano-well of the subset of the plurality of nano-wells. In specific embodiments, the signal or the change thereof is indicative of (i) the presence of a target cell in the particular nano-well, or (ii) the presence of a biomolecule produced by the target cell in the particular nano-well. In certain embodiments, the biomolecule described herein is an antibody, a monoclonal antibody, a biosimilar, a virus, a protein, a nucleotide, a biomarker, or a metabolite.

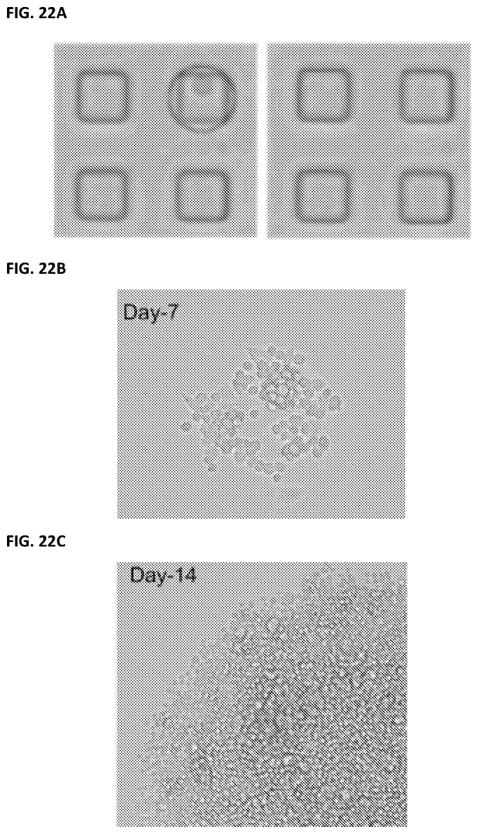

In some embodiments, the detecting described herein comprises cell morphology imaging, near-infrared imaging, fluorescence imaging, luminescence imaging, or a combination thereof. In some specific embodiments, as seen in FIG. 22A, the detection comprises an image confirmation of the presence of a single CHO cell. In some specific embodiments, as described in Example 11, the detecting step described herein comprises cell morphology imaging with parameters for high throughput.

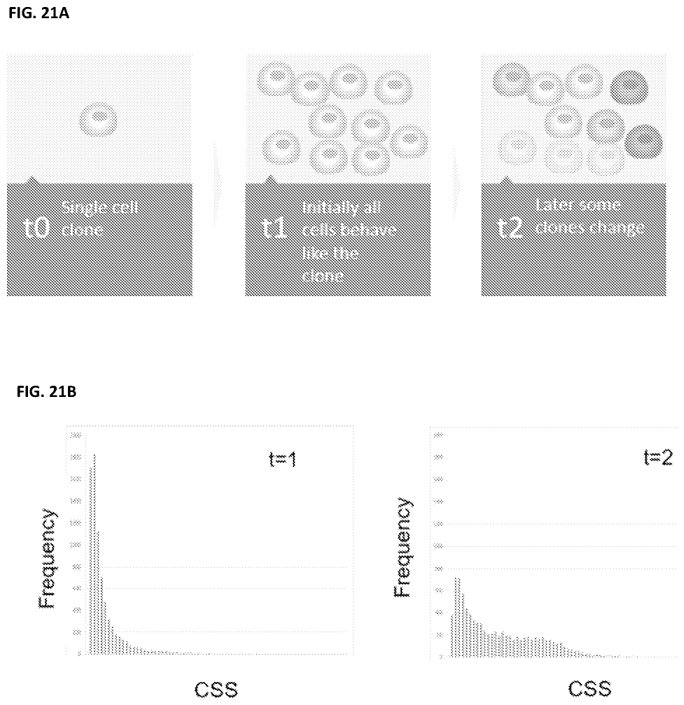

The detecting step described herein could take place at multiple different time points throughout the process described herein. In some embodiments, the detecting step described herein takes place after placing the plurality of cells into the plurality of nano-wells, but before any treatment, modifications or manipulations. In some embodiments, the detecting step described herein takes place after treating the individual nano-well with one or more regents. In some embodiments, the detecting step described herein takes place after treating the individual nano-well with the plurality of secondary cells. In some embodiments, the detecting step described herein takes place after applying a membrane to the individual nano-well to form the individual membrane-modified nano-well. In some embodiments, the detecting step described herein takes place after contacting the individual nano-well with the capture substrate. In some embodiments, the detecting step described herein takes place after contacting the individual nano-well with the secondary cell-immobilized capture substrate. In some embodiments, the detecting step described herein takes place before selecting the target cell in the particular nano-well. In some embodiments, the detecting step described herein takes place after selecting the target cell in the particular nano-well. In some embodiments, the detecting step described herein takes place at any random time points for gathering information for clonal changes throughout time, as illustrated in FIGS. 21A-21B.

In some embodiments, the step described herein is performed while the plurality of cells receive reduced perturbations when compared with corresponding perturbations received by a comparative plurality of cells in a cell line development process of (i) limiting dilution selection, (ii) fluorescence-activated cell sorting (FACS), (iii) isolating individual cells with cloning cylinders, or (iv) flow cytometry. In some embodiments, the perturbation described herein is biological perturbation with regard to the cells or the solution/environment of the cells. In other embodiments, the perturbation described herein is chemical perturbation with regard to the cells or the solution/environment of the cells. In other embodiments, the perturbation described herein is mechanical perturbation with regard to the cells or the solution/environment of the cells.

In specific embodiments, the reduced perturbations described herein are more stable and/or more optimal pH value for the plurality of cells in the plurality of nano-wells during the detecting step described herein. In specific embodiments, the reduced perturbations described herein are more stable and/or more optimal osmolarity value for the plurality of cells in the plurality of nano-wells during the detecting step described herein. In specific embodiments, the reduced perturbations described herein are more stable and/or more optimal temperature for the plurality of cells in the plurality of nano-wells during the detecting step described herein. In specific embodiments, the reduced perturbations described herein are more stable and/or more optimal humidity for the plurality of cells in the plurality of nano-wells during the detecting step described herein. In specific embodiments, the reduced perturbations described herein are more stable and/or more optimal ingredients of cell culture media for the plurality of cells in the plurality of nano-wells during the detecting step described herein. In specific embodiments, the reduced perturbations described herein are less mechanical compression for the plurality of cells in the plurality of nano-wells during the detecting step described herein. In specific embodiments, the reduced perturbations described herein are less hydrostatic pressure for the plurality of cells in the plurality of nano-wells during the detecting step described herein. As a result, in some embodiments, the detecting step described herein does not alter or change little the morphology of the plurality of cells. In some embodiments, the detecting step described herein does not alter or change little the migration of the plurality of cells. In some embodiments, the detecting step described herein does not alter or change little the growth rate of the plurality of cells. In some embodiments, the detecting step described herein does not alter or change little the expression of external-stress-sensing gene(s) in the plurality of cells.

### Selecting the Target Cell

In some embodiments, the methods described herein comprise selecting the target cell in the particular nano-well from the plurality of cells at least based on a pre-determined value of the signal or the change thereof from the detecting step described herein.

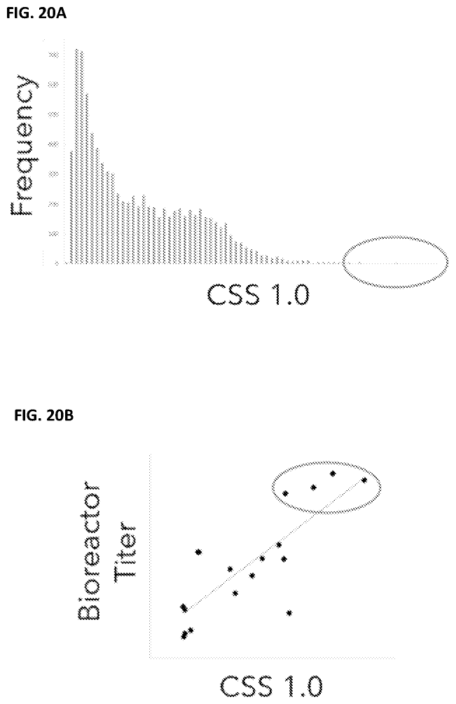

In certain embodiments, the selecting step described herein comprises predicting an expected product titer of the clone that is expanded from the target cell based on the signal or the change thereof from the detecting step described herein. In specific embodiments, as seen in FIGS. 20A-20B, predicting with high accuracy the performance of the clones enables selecting the high preforming champion factories with high efficiency, circled clones in FIGS. 20A-20B, and excluding the vast majority of underperforming cells (e.g., with low CSS values in FIG. 20A). In specific embodiments, the expected product titer of the clone correlates with an observed product titer of the clone, as exemplified in FIG. 20B.

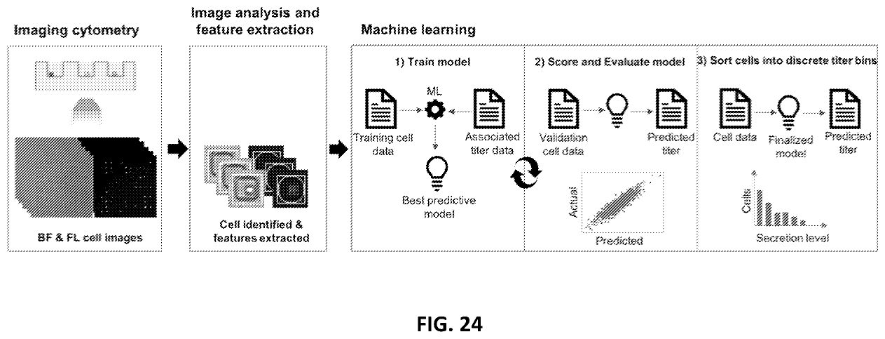

In certain embodiments, the selecting step described herein comprises performing a machine learning-based process of analyzing (i) the signal or the change thereof, and/or (ii) an additional signal or a change thereof obtained from the clone expanded from the target cell. In specific embodiments, the machine learning-based process comprises analyzing the cell morphology imaging against an optimized machine learning model built on correlating cell morphological features of selected single cells with the corresponding product quality attribute parameters of the cell cultures derived from the selected single cells, as exemplified in FIG. 24.

The duration spent from placing the plurality of cells into the plurality of nano-wells to the selecting step described herein is relatively short. In some embodiments, it is faster than when a comparative clone is obtained by (i) limiting dilution selection, (ii) fluorescence-activated cell sorting (FACS), (iii) isolating individual cells with cloning cylinders, or (iv) flow cytometry. In some embodiments, it is done in no more than 20, 19, 18, 17, 16, 15, 14, 13, 12, or 11 hour. In some embodiments, it is done in no more than 10, 9, 8, 7, 6, 5, 4, 3, 2, 1 hour, or 30 minutes. In some embodiments, it is done in no more than 20, 15, 10, 5, or 1 minute(s). In some embodiments, it is done in 10 to 9 hours, from 9 to 8 hours, from 8 to 7 hours, from 7 to 6 hours, from 6 to 5 hours, from 5 to 4 hours, from 4 to 3 hours, from 3 to 2 hours, from 2 to 1 hour(s), from 60 to 30 minutes, and from 30 to 1 minute(s). In some embodiments, it is done in about 20, 19, 18, 17, 16, 15, 14, 13, 12, or 11 hour. In some embodiments, it is done in about 10, 9, 8, 7, 6, 5, 4, 3, 2, 1 hour, or 30 minutes. In some embodiments, it is done in about 20, 15, 10, 5, or 1 minute(s).

The duration spent from exposing, treating, modifying step described herein to the selecting step described herein is relatively short. In some embodiments, it is faster than when a comparative clone is obtained by (i) limiting dilution selection, (ii) fluorescence-activated cell sorting (FACS), (iii) isolating individual cells with cloning cylinders, or (iv) flow cytometry. In some embodiments, it is done no more than 30, 20, 15, 10, or 5 minutes. In some embodiments, it is done in about 30, 20, 15, 10, or 5 minutes. In some embodiments, it is done from 30 to 5 minutes, from 20 to 5 minutes, from 15 to 5 minutes, from 10 to 5 minutes.

The duration spent in the selecting step described herein is relatively short. In some embodiments, it is faster than when a comparative clone is obtained by (i) limiting dilution selection, (ii) fluorescence-activated cell sorting (FACS), (iii) isolating individual cells with cloning cylinders, or (iv) flow cytometry. In some embodiments, it is done no more than 10, 9, 8, 7, 6, 5, 4, 3, 2, 1 minute(s), 30, or 1 second(s). In some embodiments, it is done in about 10, 9, 8, 7, 6, 5, 4, 3, 2, 1 minute(s), 30, or 1 second(s). In some embodiments, it is done from 10 to 9 minutes, from 9 to 8 minutes, from 8 to 7 minutes, from 7 to 6 minutes, from 6 to 5 minutes, from 5 to 4 minutes, from 4 to 3 minutes, from 3 to 2 minutes, from 2 to 1 minute(s), from 60 to 30 seconds, and from 30 to 1 second(s).

In some embodiments, the target cell is not removed from the particular nano-well before treating the individual nano-well with one or more reagents. In some embodiments, the target cell is not removed from the particular nano-well before treating the individual nano-well with a plurality of secondary cells. In some embodiments, the target cell is not removed from the particular nano-well before applying the membrane to the individual nano-well to form the individual membrane-modified nano-well. In some embodiments, the target cell is not removed from the particular nano-well before contacting the individual nano-well with the capture substrate. In some embodiments, the target cell is not removed from the particular nano-well before contacting the individual nano-well with the secondary cell-immobilized capture substrate. In some embodiments, the target cell is not removed from the particular nano-well before detecting the signal or a change thereof from the particular nano-well. In some embodiments, the target cell is not removed from the particular nano-well before selecting the target cell in the particular nano-well from the plurality of cells.

### Expanding the Target Cell in a Cultivation Vessel

In some embodiments, the methods described herein further comprise transferring the target cell based on selecting step described herein to a cultivation vessel, and expanding the target cell into a clone in the cultivation vessel.

Any suitable cultivation vessel or culture vessel can be adapted to culture single cells in accordance with the present disclosure. For example, vessels having a suitable for matrix attachment include tissue culture plates (including multi-well plates), pre-coated (e.g., gelatin-pre-coated) plates, T-flasks, roller bottles, gas permeable containers, and bioreactors. To increase efficiency and cell density, vessels (e.g., stirred tanks) that employ suspended particles (e.g., plastic beads or other microcarriers) that can serve as a substrate for attachment of feeder cells or an extracellular matrix can be employed. In other embodiments, undifferentiated stem cells can be cultured in suspension by providing the matrix components in soluble form. As will be appreciated, fresh medium can be introduced into any of these vessels by batch exchange (replacement of spent medium with fresh medium), fed-batch processes (i.e., fresh medium is added without removal of spent medium), or ongoing exchange in which a proportion of the medium is replaced with fresh medium on a continuous or periodic basis.

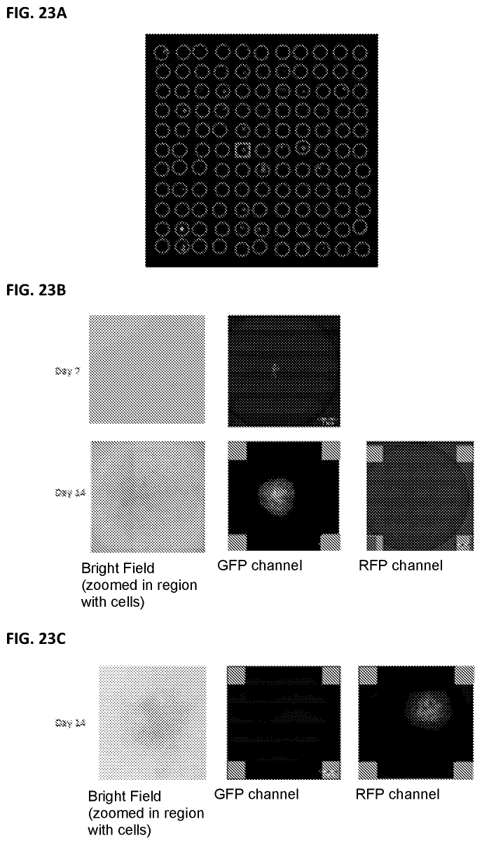

In some embodiments, the clone expanded from the target cell described herein displays higher monoclonality assurance when compared with a comparative clone obtained by (i) limiting dilution selection, (ii) fluorescence-activated cell sorting (FACS), (iii) isolating individual cells with cloning cylinders, or (iv) flow cytometry. In specific embodiments, as illustrated in FIGS. 22A-22C, tracking the target cell with high precision throughout the entire process ensures monoclonality. In specific embodiments, as exemplified by FIGS. 23A-23C, the target cell that is intended to be selected and to be expanded (e.g., GFP expressing cells in this particular example) is not mixed with other non-target cells (e.g., RFP expressing cells in this particular example). Therefore, high monoclonality is assured.

In some embodiments, the clone expanded from the target cell described herein displays higher viability when compared with a comparative clone obtained by (i) limiting dilution selection, (ii) fluorescence-activated cell sorting (FACS), (iii) isolating individual cells with cloning cylinders, or (iv) flow cytometry. In specific embodiments, as exemplified in FIGS. 22A-22C, high viability more after recovery is achieved. In certain embodiments, more than 10%, 20% or 30% viability after recovery is achieved. In other embodiments, more than 40%, 50%, 60%, or 70% viability after recovery is achieved. In other embodiments, more than 80%, 90%, 95%, or 99% viability after recovery is achieved.

In some embodiments, as seen in FIGS. 21A-21B, clones undergo phenotypic shifts over time, which present potential bio-manufacturing issues. Accordingly, in some embodiments, the methods described herein further comprise monitoring the clonal changes over time. In other embodiments, the methods described herein further comprise real-time optimization based on the information from monitoring the clonal changes over time,

### Q. A Method for Facilitating Clone Selection of a Cell Line

In some embodiments, as seen in FIG. 24, a machine-learning based approach could be utilized to facilitate clone selection of a cell line.

### Generating Images

In some embodiments, the methods for facilitating clone selection of a cell line comprises generating, by an imaging unit, a first plurality of images of each of the plurality of candidate single cells individually. In certain embodiments, each of the plurality of candidate single cells resides in an individual nano-well of a plurality of nano-wells.

Various types of images could be generated based on specific scenarios of clone selection. In some embodiments, the methods described herein comprises generating cell morphology imaging. In some embodiments, the methods described herein comprises generating near-infrared imaging. In some embodiments, the methods described herein comprises generating fluorescence imaging. In some embodiments, the methods described herein comprises generating luminescence imaging. In some embodiments, the methods described herein comprises generating a combination of all or part of the above imaging.

### Detecting Morphological Cell Features

In some embodiments, the methods for facilitating clone selection of a cell line comprises detecting, by one or more processors analyzing the first plurality of images for each of the plurality of candidate single cells, one or more morphological cell features of each of the plurality of candidate single cells depicted in the first plurality of images.

In some embodiments, before extracting morphological cell features from images, binarization is applied to pre-process the images. In some embodiments, before extracting morphological cell features from images, thresholding is applied to pre-process the images. In some embodiments, before extracting morphological cell features from images, resizing is applied to pre-process the images. In some embodiments, before extracting morphological cell features from images, normalization is applied to pre-process the images. In some embodiments, before extracting morphological cell features from images, local sampling of mini patches is applied to pre-process the images. In some embodiments, before extracting morphological cell features from images, a combination of the above is applied to pre-process the images.

In some embodiments, a morphological cell feature of interest is shape. In some embodiments, a morphological cell feature of interest is size. In some embodiments, a morphological cell feature of interest is color. In some embodiments, a morphological cell feature of interest is pattern. In some embodiments, a morphological cell feature of interest is texture. In some embodiments, a morphological cell feature of interest is nucleus size. In some embodiments, a morphological cell feature of interest is organelles. In some embodiments, a morphological cell feature of interest is a combination of all or part of the above.

In some embodiments, feature extraction is performed from the above-described morphological cell features. In specific embodiments, gray level co-occurrence Matrix is extracted. In specific embodiments, local binary pattern is extracted. In other specific embodiments, features are simultaneously optimized in deep learning.

In some embodiments, the data processing described herein is performed on cloud servers. In some embodiments, the data processing described herein is performed on in-house servers. In some embodiments, the data processing described herein is performed on both cloud and in-house servers.

### Determining Predicted Quality Attributes for an Expanded Colony

In some embodiments, the methods for facilitating clone selection of a cell line comprises based on the one or more morphological cell features, determining, by the one or more processors and according to a finalized single cell-to-colony machine learning model, one or more predicted quality attributes for a colony expanded from each of the plurality of candidate single cells. In specific embodiments, the finalized single cell-to-colony model predicts quality attributes of a hypothetical colony based on at least the one or more morphological cell features of a single cell.

In some embodiments, the predicted quality attribute for the colony is titer. In some embodiments, the predicted quality attribute for the colony is cell growth metric. In some embodiments, the predicted quality attribute for the colony is viable cell density. In some embodiments, the predicted quality attribute for the colony is characteristics. In some embodiments, the predicted quality attribute for the colony is expression of surface glycoproteins. In some embodiments, the predicted quality attribute for the colony is glycosylation. In some embodiments, the predicted quality attribute for the colony is phosphorylation. In some embodiments, the predicted quality attribute for the colony is deamidation. In some embodiments, the predicted quality attribute for the colony is methylation. In some embodiments, the predicted quality attribute for the colony is acetylation aggregation. In some embodiments, the predicted quality attribute for the colony is monoclonality. In some embodiments, the predicted quality attribute for the colony is expression of cell markers. In some embodiments, the predicted quality attribute for the colony is biological activities. In some embodiments, the predicted quality attribute for the colony is impurities. In some embodiments, the predicted quality attribute for the colony is a combination of all or part of the above.

In some embodiments, for classification type of attributes, the single-cell-to-colony machine learning model described herein is multiclass logistic regression or a derivative thereof. In some embodiments, for classification type of attributes, the single-cell-to-colony machine learning model described herein is multiclass boosted decision tree or a derivative thereof. In some embodiments, for classification type of attributes, the single-cell-to-colony machine learning model described herein is neural network algorithms or a derivative thereof.

In some embodiments, for numerical type of attributes, the single-cell-to-colony machine learning model described herein is linear regression or a derivative thereof. In some embodiments, for numerical type of attributes, the single-cell-to-colony machine learning model described herein is neural network algorithms or a derivative thereof.

In some embodiments, the finalized single cell-to-colony model is optimized by using a training data set comprising (i) the one or more morphological cell features from a second plurality of images for a plurality of training single cells, and (ii) measured quality attributes of each colony expanded from each of the plurality of training single cells. In other embodiments, the finalized single cell-to-colony model is further optimized by (a) using a validation data set comprising (i) the one or more morphological cell features from a third plurality of images for a plurality of validation single cells, and (ii) measured quality attributes of each colony expanded from each of the plurality of validation single cells, and (b) comparing one or more predicted quality attributes of each of the plurality of validation single cells with the measured quality attributes of each of the colony expanded from each of the plurality of validation single cells.

### Ranking the Candidate Single Cells Based on Predicted Quality Attributes

In some embodiments, the methods for facilitating clone selection of a cell line comprises ranking the plurality of candidate single cells according to the one or more predicted quality attributes for each of the plurality of candidate single cells.

In specific embodiments, the ranking is sorting the candidate single cells into different categories based on classification type of predicted quality attributes. In certain special embodiment, the categories are set to be low/mid/high, fast/mid/slow, or preferred/mediocre/less preferred. In other specific embodiments, the ranking is sorting the candidate single cells into different tiers based on numerical type of predicted quality attributes.

### III. Systems

### A. Integrated System

In some embodiments described herein, an integrated system for high throughput cell an array of nano-wells configured for containing individual target cells of a plurality of target cells; b) one or more fluidics modules configured for delivery of one or more reagents to the plurality of target cells; c) a detection module configured for performing secretion assay and performing direct measurements of the individual cells; d) a cell recovery apparatus configured for recovery of a target cell of the individual cells; wherein the system is configured to reach a decision for selecting the target cell for recovery within 3 hours from initialization of the system; and wherein the system is configured to yield clones with a mean productivity of greater than 5 grams per liter.

In certain embodiments of the method, the time to reach a decision for selecting the target cell does not exceed 1 hour from initialization of the method to 15 hours from initialization of the method. In certain embodiments of the method, the time to reach a decision for selecting the target cell does not exceed 1 hour from initialization of the method to 2 hours from initialization of the method, 1 hour from initialization of the method to 4 hours from initialization of the method, 1 hour from initialization of the method to 5 hours from initialization of the method, 1 hour from initialization of the method to 10 hours from initialization of the method, 1 hour from initialization of the method to 15 hours from initialization of the method, 2 hours from initialization of the method to 4 hours from initialization of the method, 2 hours from initialization of the method to 5 hours from initialization of the method, 2 hours from initialization of the method to 10 hours from initialization of the method, 2 hours from initialization of the method to 15 hours from initialization of the method, 4 hours from initialization of the method to 5 hours from initialization of the method, 4 hours from initialization of the method to 10 hours from initialization of the method, 4 hours from initialization of the method to 15 hours from initialization of the method, 5 hours from initialization of the method to 10 hours from initialization of the method, 5 hours from initialization of the method to 15 hours from initialization of the method, or 10 hours from initialization of the method to 15 hours from initialization of the method. In certain embodiments of the method, the time to reach a decision for selecting the target cell does not exceed 1 hour from initialization of the method, 2 hours from initialization of the method, 4 hours from initialization of the method, 5 hours from initialization of the method, 10 hours from initialization of the method, or 15 hours from initialization of the method. In certain embodiments of the method, the time to reach a decision for selecting the target cell does not exceed at least 1 hour from initialization of the method, 2 hours from initialization of the method, 4 hours from initialization of the method, 5 hours from initialization of the method, or 10 hours from initialization of the method. In certain embodiments of the method, the time to reach a decision for selecting the target cell does not exceed at most 2 hours from initialization of the method, 4 hours from initialization of the method, 5 hours from initialization of the method, 10 hours from initialization of the method, or 15 hours from initialization of the method. In certain embodiments, wherein the system comprises an apparatus configured for reversible sealing a capture substrate to the array of nano-wells, whereupon sealing a substantially aligned and substantially fluid tight seal between the one or more capture substrates and the one or more array of nano-wells is made

In certain embodiments, the direct measurements comprise bright field microscopy measurements. In certain embodiments, the direct measurements comprise fluorescence microscopy measurements. In certain embodiments, the system comprises a controller configured for actuating the system and analyzing data.

### B. Array of Nano-Wells

In certain embodiments, the array of nano-wells is comprised of a hard material. In certain embodiments, the array of nano-wells is comprised of a soft material. In certain embodiments, the hard material comprises a transparent plastic or a transparent glass material, or a reflective material. In certain embodiments, the soft material comprises a transparent elastomeric material.

In certain instances, a nano-well of the array of nano-wells has a diameter of about 5 microns to about 175 microns. In certain instances, a nano-well of the array of nano-wells has a diameter of about 5 microns to about 50 microns, about 5 microns to about 100 microns, about 5 microns to about 150 microns, about 5 microns to about 175 microns, about 50 microns to about 100 microns, about 50 microns to about 150 microns, about 50 microns to about 175 microns, about 100 microns to about 150 microns, about 100 microns to about 175 microns, or about 150 microns to about 175 microns. In certain instances, a nano-well of the array of nano-wells has a diameter of about 5 microns, about 50 microns, about 100 microns, about 150 microns, or about 175 microns. In certain instances, a nano-well of the array of nano-wells has a diameter of at least about 5 microns, about 50 microns, about 100 microns, or about 150 microns. In certain instances, a nano-well of the array of nano-wells has a diameter of at most about 50 microns, about 100 microns, about 150 microns, or about 175 microns.

In certain embodiments, the center to center spacing for nano-wells in the array of nano-wells is 10 microns to 200 microns. In certain embodiments, the center to center spacing for nano-wells in the array of nano-wells is 10 microns to 50 microns, 10 microns to 100 microns, 10 microns to 150 microns, 10 microns to 200 microns, 50 microns to 100 microns, 50 microns to 150 microns, 50 microns to 200 microns, 100 microns to 150 microns, 100 microns to 200 microns, or 150 microns to 200 microns. In certain embodiments, the center to center spacing for nano-wells in the array of nano-wells is 10 microns, 50 microns, 100 microns, 150 microns, or 200 microns. In certain embodiments, the center to center spacing for nano-wells in the array of nano-wells is at least 10 microns, 50 microns, 100 microns, or 150 microns. In certain embodiments, the center to center spacing for nano-wells in the array of nano-wells is at most 50 microns, 100 microns, 150 microns, or 200 microns.

In some embodiments, a nano-well of the array of nano-wells has a depth of 15 microns to 250 microns. In some embodiments, a nano-well of the array of nano-wells has a depth of 15 microns to 25 microns, 15 microns to 50 microns, 15 microns to 100 microns, 15 microns to 150 microns, 15 microns to 200 microns, 15 microns to 250 microns, 25 microns to 50 microns, 25 microns to 100 microns, 25 microns to 150 microns, 25 microns to 200 microns, 25 microns to 250 microns, 50 microns to 100 microns, 50 microns to 150 microns, 50 microns to 200 microns, 50 microns to 250 microns, 100 microns to 150 microns, 100 microns to 200 microns, 100 microns to 250 microns, 150 microns to 200 microns, 150 microns to 250 microns, or 200 microns to 250 microns. In some embodiments, a nano-well of the array of nano-wells has a depth of 15 microns, 25 microns, 50 microns, 100 microns, 150 microns, 200 microns, or 250 microns. In some embodiments, a nano-well of the array of nano-wells has a depth of at least 15 microns, 25 microns, 50 microns, 100 microns, 150 microns, or 200 microns. In some embodiments, a nano-well of the array of nano-wells has a depth of at most 25 microns, 50 microns, 100 microns, 150 microns, 200 microns, or 250 microns.

In some embodiments, a nano-well of the array of nano-wells has a diameter to depth ratio of 0.1 to 6. In some embodiments, a nano-well of the array of nano-wells has a diameter to depth ratio of 0.1 to 0.2, 0.1 to 1, 0.1 to 1, 0.1 to 2, 0.1 to 4, 0.1 to 6, 0.2 to 1, 0.2 to 1, 0.2 to 2, 0.2 to 4, 0.2 to 6, 1 to 1, 1 to 2, 1 to 4, 1 to 6, 1 to 2, 1 to 4, 1 to 6, 2 to 4, 2 to 6, or 4 to 6. In some embodiments, a nano-well of the array of nano-wells has a diameter to depth ratio of 0.1, 0.2, 1, 1, 2, 4, or 6. In some embodiments, a nano-well of the array of nano-wells has a diameter to depth ratio of at least 0.1, 0.2, 1, 1, 2, or 4. In some embodiments, a nano-well of the array of nano-wells has a diameter to depth ratio of at most 0.2, 1, 1, 2, 4, or 6.

In some embodiments, the well has a volume of 10 picoliters to 2,000 picoliters. In some embodiments, the well has a volume of 10 picoliters to 100 picoliters, 10 picoliters to 250 picoliters, 10 picoliters to 500 picoliters, 10 picoliters to 1,000 picoliters, 10 picoliters to 1,500 picoliters, 10 picoliters to 2,000 picoliters, 100 picoliters to 250 picoliters, 100 picoliters to 500 picoliters, 100 picoliters to 1,000 picoliters, 100 picoliters to 1,500 picoliters, 100 picoliters to 2,000 picoliters, 250 picoliters to 500 picoliters, 250 picoliters to 1,000 picoliters, 250 picoliters to 1,500 picoliters, 250 picoliters to 2,000 picoliters, 500 picoliters to 1,000 picoliters, 500 picoliters to 1,500 picoliters, 500 picoliters to 2,000 picoliters, 1,000 picoliters to 1,500 picoliters, 1,000 picoliters to 2,000 picoliters, or 1,500 picoliters to 2,000 picoliters.

In some embodiments, the well has a volume of 10 picoliters, 100 picoliters, 250 picoliters, 500 picoliters, 1,000 picoliters, 1,500 picoliters, or 2,000 picoliters. In some embodiments, the well has a volume of at least 10 picoliters, 100 picoliters, 250 picoliters, 500 picoliters, 1,000 picoliters, or 1,500 picoliters. In some embodiments, the well has a volume of at most 100 picoliters, 250 picoliters, 500 picoliters, 1,000 picoliters, 1,500 picoliters, or 2,000 picoliters.

In some embodiments, the well comprises shapes of circle, square, triangle, diamond or other shapes that enable fluid dynamic control.

In certain embodiments, the number of nano-wells per array is about 1,000 to about 10,000,000. In certain embodiments, the number of nano-wells per array is about 1,000 to about 100,000, about 1,000 to about 1,000,000, about 1,000 to about 10,000,000, about 100,000 to about 1,000,000, about 100,000 to about 10,000,000, or about 1,000,000 to about 10,000,000. In certain embodiments, the number of nano-wells per array is about 1,000, about 100,000, about 1,000,000, or about 10,000,000. In certain embodiments, the number of nano-wells per array is at least about 1,000, about 100,000, or about 1,000,000. In certain embodiments, the number of nano-wells per array is at most about 100,000, about 1,000,000, or about 10,000,000.

In some embodiments, the number of cells per a nano-well of an array of nano-wells is 0 to 1,000. In some embodiments, the number of cells per a nano-well of an array of nano-wells is 0 to 1, 0 to 2, 0 to 5, 0 to 10, 0 to 100, 0 to 1,000, 1 to 2, 1 to 5, 1 to 10, 1 to 100, 1 to 1,000, 2 to 5, 2 to 10, 2 to 100, 2 to 1,000, 5 to 10, 5 to 100, 5 to 1,000, 10 to 100, 10 to 1,000, or 100 to 1,000. In some embodiments, the number of cells per a nano-well of an array of nano-wells is 0, 1, 2, 5, 10, 100, or 1,000. In some embodiments, the number of cells per a nano-well of an array of nano-wells is at least 0, 1, 2, 5, 10, or 100. In some embodiments, the number of cells per a nano-well of an array of nano-wells is at most 1, 2, 5, 10, 100, or 1,000.

### C. Plate

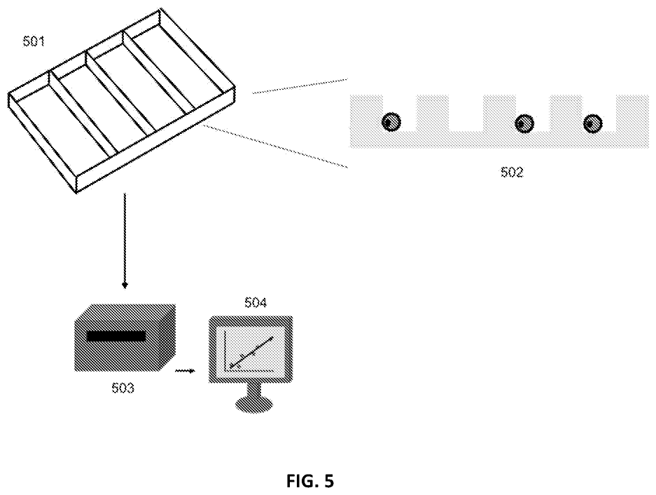

Described herein, in some embodiments, is a method of analysis using a plate (501) with four large wells as shown in FIG. 5. In some embodiments, the bottom surface of each large well (502, 503) comprises many individual nano-wells (504, 505), which contain a single or a few cells. In some embodiments, the plate (501) is inserted into a plate reader (506) for detection. In some embodiments, data is then analyzed and used for selection of individual cells for recovery and further analysis.

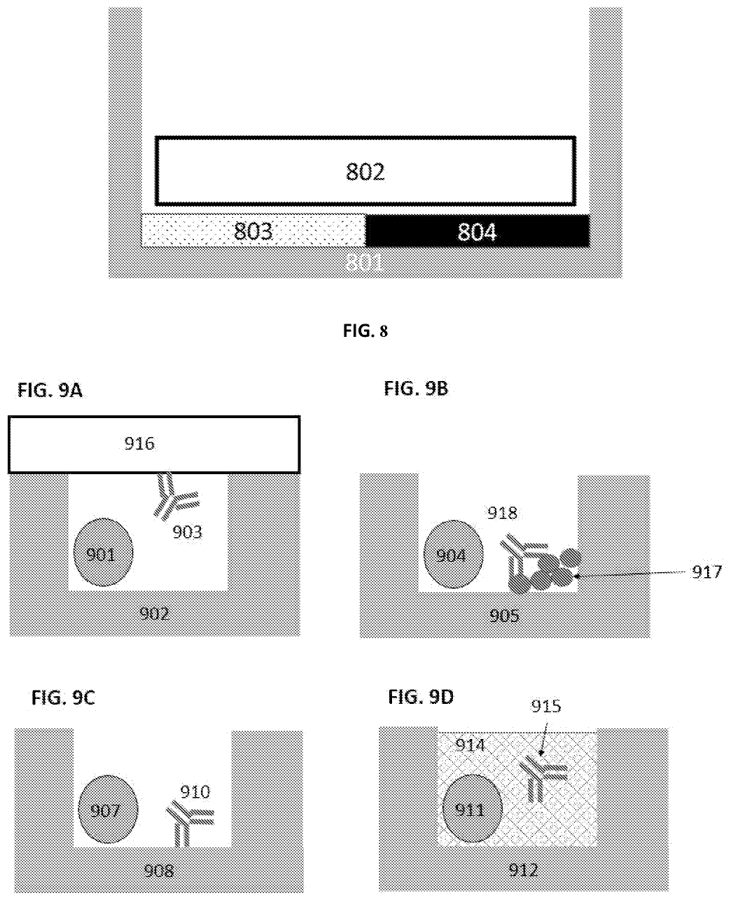

In certain embodiments, a plate comprises a plurality of the array of nano-wells. In certain embodiments, the plate comprises a plurality of recesses. In certain embodiments, a recess of the plurality of recesses comprises an array of nano-wells. In some embodiments, as seen in FIG. 8 which shows a cross section of large well of a plate (801), the plate is made with a hard plastic and a capture substrate (802) made of a soft material. In this embodiment, the hard plastic is transparent. A capture substrate is placed over the bottom of the large well to seal the nano-wells located there. In some instances, the surface of the large well (801) are treated with a non-stick agent (803) or a capture agent (804).

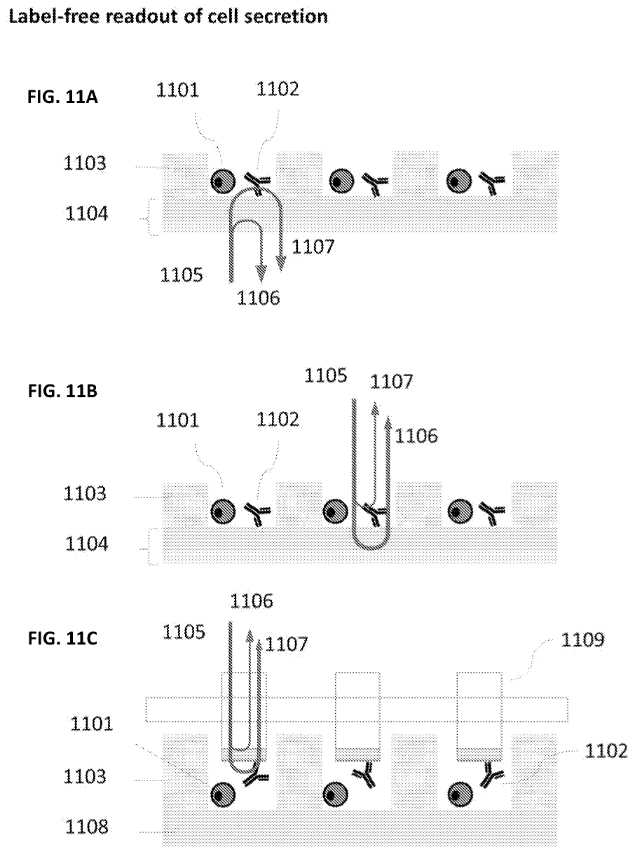

In some embodiments, the plate comprises a plurality of recesses. In other embodiments, a recess of the plurality of recesses comprises an array of nano-wells. The recesses are also known as large wells, on the order of 25 mm in width, 75 mm in length and 10 mm in depth. In some embodiments, the bottom surface of the large wells contains arrays of nano-wells fabricated into the plate. In some embodiments, as shown in cross section view in FIG. 10A, the plate (1003) comprises a flow cell where an inlet line (1001) fluidically connects a cavity created by sealing a substrate (1002) to the array of nano-wells (1005). In some embodiments, the array of nano-wells which is embedded into the bottom of a large well (1004) of the plate (1003). In some embodiments, the flow cell allows for fluidic connection to the reagents and samples via the inlet line (1001) as well as a waste receptacle or back to the reagents, via an outlet (1007) as shown in the top down view of FIG. 10B. In some embodiments, the plate comprises 2 large wells. In some embodiments, the plate comprises 4 large wells. In some embodiments, the plate comprises 6 large wells. In some embodiments, the plate comprises 8 large wells. In some embodiments, the plate comprises 12 large wells. In some embodiments, the plate comprises 24 large wells. In some embodiments, the plate comprises 48 large wells. In some embodiments, the plate comprises 96 large wells. In some embodiments, the plate comprises 4 large wells. FIG. 10C shows another cross-sectional view of the flow cell rotated 90 degrees from the viewing angle of FIG. 10A. Additionally, in some embodiments, as seen in FIG. 10C a gap or cavity is formed between the substrate (1002) and the array of nano-wells (1005) to allow for flow of reagents and wash buffers.

### D. Capture Substrate

In certain aspects, FIG. 1 shows a capture substrate (104) for use in secretion profiling, imaging, and subsequent analytics of the single cells. In some embodiments, a capture layer (105) captures the secreted biomolecule (106). In certain aspects, assay reagents (107, 109) are added to the system. In some embodiments, after analysis, an identified single cell is recovered (108) from the nano-well chip (103).

In certain circumstances the capture substrate are 25×75×1 mm glass slide that is coated with a capture agent, or capture layer, designed to capture secreted biomolecules from cells within the wells of the array of nano-wells. In some embodiments, the capture substrate is reversibly sealed against the array of nano-wells to create a fluid tight seal that isolates the cell from other cells.

In certain circumstances the capture substrate captures secreted biomolecules from the cell after sealing. In these circumstances, when the capture substrate is removed from the array of nano-wells an imprint, or a collection of one or more biomolecules secreted by the cell are captured on the surface of the capture substrate at locations that correspond to a well of the array of nano-wells. The indexed relationship between the location of isolated secreted biomolecules on the surface of the capture substrate and the isolated cell or cells in the nano-well of the array of nano-wells allows for one to correlate secretion characteristics to the cell or cells in the corresponding well. This, in turn allows for the recovery of the cell or cells from the well. In some embodiments, once recovered, the cells are studied for their unique secretion aspects.

In some embodiments the capture substrate is made of a transparent hard plastic material. In other embodiments, the capture substrate is made of a soft transparent elastomeric material, such as polydimethylsiloxane, or PDMS.

In certain instances, placing the capture agent on the capture substrate allows for the cell to not be disturbed by detection reagents, allowing for a gentler process.

In certain embodiments, the capture substrate comprises a sensing surface. In certain embodiments, the array of nano-wells comprises the sensing surface. In certain embodiments, the sensing surface comprises a layered semiconductor. In certain embodiments, the sensing surface is configured for reflection mode imaging for real-time endpoint detection of binding on the sensing surface. In certain embodiments, the sensing surface is configured for surface plasmon resonance detection of the articles. In certain embodiments, the sensing surface is configured for interferometric detection of the articles. In certain embodiments, the sensing surface is configured for whispering gallery mode detection of the articles.

### E. Capture Substrate and Array Handler

In certain aspects, disclosed herein is a mechanism for placing a capture substrate in proximity to an array of nano-wells, comprising: a top piece configured to immobilize capture substrate; a base configured to immobilize an array of nano-wells; wherein the base comprises one or more alignment rods to align the top piece to the base such that the capture substrate and the array of nano-wells are fixed in a coplanar orientation; and wherein a distance between the capture substrate and the array of nano-wells are controllably varied along an axis perpendicular to the coplanar planes of the capture substrate and the array of nano-wells to place the capture substrate and the array of nano-wells in proximity to each other.

In certain embodiments, the distance is minimized to form a seal between the capture substrate and the array of nano-wells that is substantially aligned and substantially fluid tight.

In certain embodiments, the capture substrate is aligned with the array of nano-wells, where both positioned in coplanar orientation and in close proximity, where multiple pair capture substrates are placed on one plate of multiple large wells simultaneously.

In some embodiments, one or more of the array of nano-wells are contained within a plate. In certain embodiments, wherein the plate comprises one or more recesses, each recess contains one or more of the arrays of nano-wells. In some embodiments, the plate comprises one or more recesses, wherein an array of nano-wells is placed and removed from a recess of the one or more recesses. In some embodiments, a force is applied equally across a region of the capture substrate, the array of nano-wells, or a combination of both wherein the pressure applied across the region is substantially uniform. In some embodiments, a specific force is applied equally across a region of the capture substrate, the array of nano-wells, or a combination of both wherein a predetermined pressure applied across the region is substantially uniform. In some embodiments, the recess comprises one or more channels configured to accept fluid displaced between the capture substrate and the array of nano-wells. In some embodiments, the recess comprises one or more ridges to contain and align the capture substrate relative to the array of nano-wells. In some embodiments, the recess further comprises an alignment recess configured to align the capture substrate relative to the array of nano-wells. In some embodiments, the recess or large well, contains channels configured to form a pedestal and the capture substrate also contains a recess configured to accept the pedestal and allow alignment and mating the capture substrate and the array of nano-wells. In some embodiments, the plate is in fluidic connection with one or more reservoirs wherein the one or more reservoirs contain the one or more reagents.

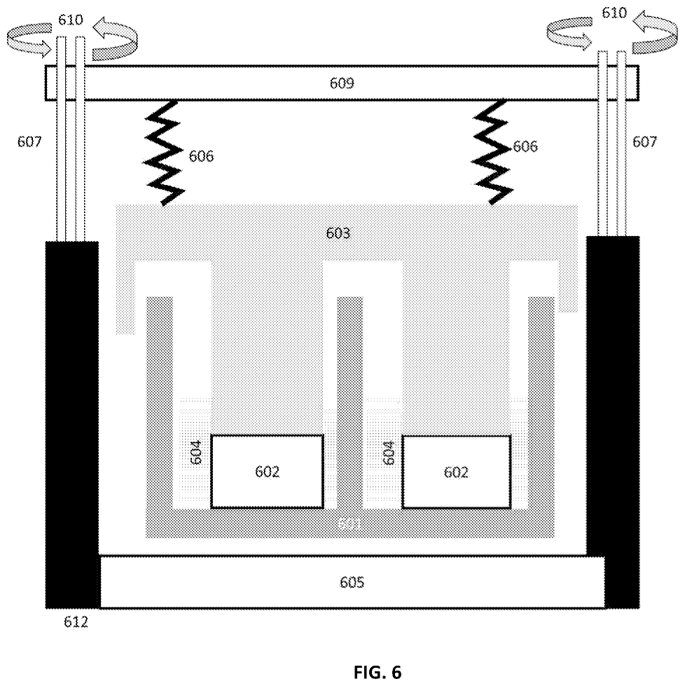

In some embodiments, as seen in FIG. 6. each large well of a plate (601) further contains arrays of nano-wells, wherein each large well is filled with media (604). In some embodiments, a capture substrate (602) is placed over each array of nano-wells at the bottom of each large well of the plate. In some embodiments, a compression member (603) is placed over the well plate and contacts the back of the capture substrates. In some embodiments, the plate (601), is placed above a transparent window (605) within the base (612) of the mechanism. In certain instances, the array of nano-wells is imaged through the window (605) and the plate (601) which is made of a transparent plastic. In these instances, two springs (606) connected to a top piece (609) are also connected to the compression member. The top piece is connected to guide rods (607) which align the top piece and compression member, which is holding the capture substrate (602), to where the capture substrate is to be placed on the plate (601). Once initiated, the assembly of the top piece (609), the compression member (603) and the capture substrates (602) are lowered and placed in contact with the plate (601) in a rotationally aligned and coplanar fashion. When pressed into place, a fluid-tight seal is formed.

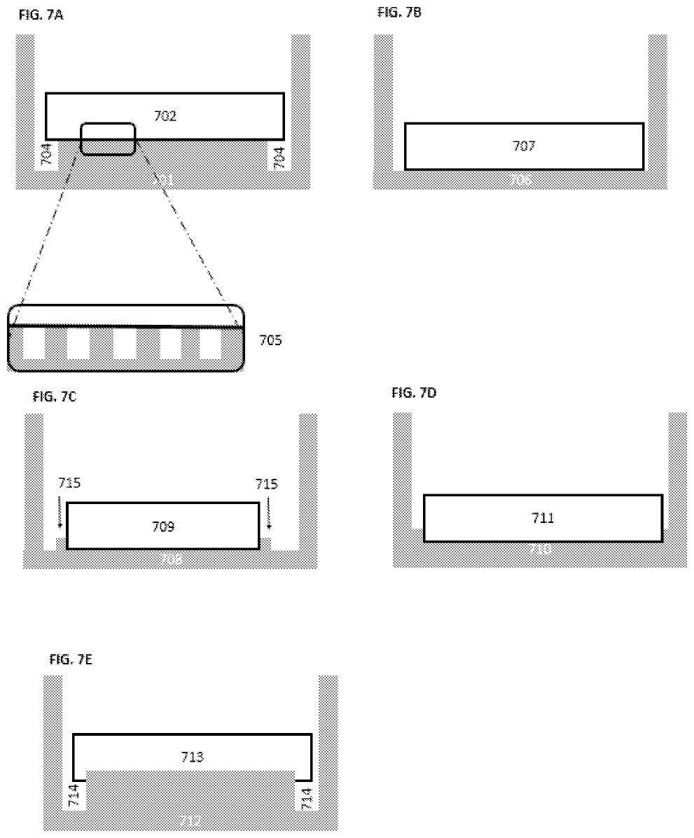

In some embodiments, as shown in FIGS. 7A-7E, a system is used to contact the nano-well plate with the capture substrate. In one embodiment, seen in FIG. 7A, a large well of the nano-well plate (701) has channels (704) to prevent hydroplaning when the capture substrate (702) is placed on the nano-well plate. The inset (705) shows how the bottom of each large well of the plate (701) comprises a plurality of wells. In another embodiment, seen in FIG. 7B, the plate (706) and the capture substrate (707) are flat against each other. In another embodiment, seen in FIG. 7C the plate (708) has two ridges (715) for alignment. In another embodiment, seen in FIG. 7D, there is a recess in the plate (710) for the capture substrate (711) to align with the plate. In another embodiment, seen in FIG. 7E, there are two channels (714) in the plate (712) to prevent hydroplaning. However, unlike the embodiment of FIG. 7A, in this embodiment, the capture substrate (713) has a recess to align with the plate.

### F. Reagent Module

In certain circumstances, the integrated system for high-throughput cell line development contains a reagent module to house the reagents described herein. The regent module is fluidically connected to the flow module.

### G. Fluidics

In certain circumstances described herein an integrated system contains fluidics to fluidically connect the array of nano-wells with the reagent module. In other instances, the fluidics connect to both the array of nano-wells and the capture substrate. FIG. 10. shows a system for identifying and analyzing cells comprising a capture substrate further comprising fluidics to form a flow cell when mated with the array of nano-wells of the plate. The fluidic capture substrate (1002) is placed in one large well of the plate (1001). In some embodiments, a second fluidic capture substrate (1003) is placed in another tray of the plate. An inset (1004) of one large well of the plate shows that each large well comprises a plurality of nano-wells (1006) each containing a single cell (1013-1017). A top view shows a plate (1007) comprising 4 trays (1008-1011). Fluid, media, factors, and cells, to enters through the inlet (1018) and exits through the outlet (1019). A side view shows the plate (1020) covered with the fluidic capture substrate (1021). Fluid enters through the inlet (1022) and exits through the outlet (1023). An inset (1032) shows that the large well comprises a plurality of nano-wells (1026) covered by the fluidic capture substrate (1021), each containing a single cell (1027-1031). In some embodiments, the multi-large-well plate (1001) is made of consumable plastics and contains nano-wells at the bottom of each large well. In certain instances, the large-multi-well plate uses the SBS format. The four large rectangular wells are present that fit standard microscope slide sized capture substrates, are about or greater than 25 mm wide, about or greater than 75 mm and about or greater than 10 mm deep.

As used herein, the term “nano-well” or “well” is a chamber with an opening/aperture for the introduction or removal of materials/solutions/reagents/buffers into or out of the chamber. The dimension of a nano-well can be within or close to the nanometer range, can exceed the nanometer range, and can be below the nanometer range. The nano-well or well can be on a chip or solid substrate. A chip or solid substrate can have a plurality of nano-wells or wells.

### H. Imaging Module

In certain aspects described herein, the integrated system contains an imaging module. In some embodiments, the imaging module is configured for imaging methods such as but not limited to bright field and fluorescence microscopy, interferometry for both single point and large field imaging detection, surface plasmon resonance. In certain aspects, the imaging module is also configured for end point and continuous data acquisition. In certain circumstances, the imaging module provide white light or laser excitation sources. In some embodiments, optical analytics comprises, bright field microscopy, fluorescence microscopy, laser excitation and detection with a photomultiplier tube, or a combination thereof.

### I. Cell Picker Module

In certain embodiments, selected individual target cells are recovered from the individual nano-wells by the cell picker module. In these instances, the cell picker module comprises a micromanipulated pipette system that is configured to removed selected individual cells from the wells in which they reside. In certain embodiments, the recovered cells are live cells.

### J. Controller Module

In certain aspects described herein, the integrated system for high throughput cell line development comprises a controller module for controlling at least all the modules and methods described herein. One aspect is to take instructions from the user and process them as routine methods for high-throughput cell line development as described herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “antibody” refers proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment f(including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass. In some embodiments, the antibody is an antibody mimetic. In certain embodiments, the antibody mimetic comprises an affibody, an adnectin, an affilin, an affimer, an affatin, an alphabody, an anticalin, an aptamer, an atrimer, an avimer, a fynomer, a DARPin, an armadillo repeat protein, a Kunit domain inhibitor molecule, a knottin molecule, a designated ankyrin repeat molecule, a monobody, or a nanofitin.

As used herein, the term “bispecific” refers to bispecific antibody or bispecific T-cell receptor (TCR). This term refers in some aspects to an antibody or TCR that shows specificities to two different types of antigens. The terms as used herein specifically include, without limitation, antibodies and TCRs which show binding specificity for a target antigen and to another target that facilitates delivery to a particular tissue. Similarly, multi-specific antibodies and TCRs have two or more binding specificities.

As used herein, the term “marker” or “biomarker” refers to a biological molecule, such as, for example, a nucleic acid, peptide, protein, hormone, and the like, whose presence or concentration can be detected and correlated with a known condition, such as a disease state.

The section headings used herein are for organizational purposes only and are not to be construed as limiting the subject matter described.

### EXAMPLES

The following examples are included for illustrative purposes only and are not intended to limit the scope of the invention.

### Example 1: Sterile and Automated Sealing of Capture Substrate to an Array of Nano-Wells

In this example a mechanism for sterile operation of sealing a capture substrate to an array of nano-wells is described and can be seen in FIG. 6. Each large well of a plate (601) that further contains arrays of nano-wells in each large well, is filled with media (604). A capture substrate (602) is placed over each array of nano-wells at the bottom of each large well of the plate. A compression member (603) is placed over the well plate and contacts the back of the capture substrates. The plate (601), is placed above a transparent window (605) within the base (612) of the mechanism. The array of nano-wells is imaged through the window (605) and the plate (601) which is made of a transparent plastic. Two springs (606) connected to a top piece (609) are also connected to the compression member. The top piece is connected to guide rods (607) which align the top piece and compression member, which is holding the capture substrate (602), to where the capture substrate is to be placed on the plate (601). Once initiated, the assembly of the top piece (609), the compression member (603) and the capture substrates (602) are lowered and placed in contact with the plate (601) in a rotationally aligned and coplanar fashion. When pressed into place, a fluid-tight seal is formed. FIGS. 7A-7E show a system used to contact the nano-well plate with the capture substrate. The nano-well plate (701) has channels (704) to prevent hydroplaning when the capture substrate (702) is placed on the nano-well plate. The inset (705) shows how each nano-well plate comprises a plurality of wells.

### Example 2: Label-Free Detection

In this example, as seen in FIG. 11A, label free readout of cell secretion is described. Secreted biomolecules (1102) of live single cells (1101) are captured on a layered substrate (1104) which comprises the bottom of the nano-wells. White light is illuminated onto the bottom of the onto the layered substrate (1104). The incident light is reflected off a reference layer (1106) and the biolayer composed of secretions (1107). Interferometric imaging is used for readout and relies on the interference of the light reflected from both the reference layer and the biolayer. Data is collected continuously throughout the experiment. In this example, the wells are not sealed.

### Example 3: Terminal Assay Using Reference Live Cell Array

In this example, as seen in FIG. 13, live single cells (1301) are each separated into individual nanoliter wells of a nanoliter well array (1302). The separated live single cells (1301) are grown into separate small-size colonies (1303) in each nanoliter well. Throughout the colony growth process image cytometry is performed, from the initial presence of the individual live single cells (1301) to the growth of the small-size colonies (1303). The nanoliter well array (1302) is then sealed with a capture substrate (1304), wherein some cells of the small-size colonies attach to the capture substrate (1304). The capture substrate (1304) is then separated from the nanoliter well array (1302). A terminal assay is then performed on the cells transferred (1305) to the capture substrate (1304). The results of the terminal assay of a particular cell that was transferred to the lid (1305) can be correlated to the well from which it came by matching up the positions of the transferred cells (1305) to the wells of the array of wells (1302). Therefore, the results of the terminal assay ran on an individual transferred cell (1305) can be correlated to the live, small-size colony (1303) still present in an individual well of the array of nanoliter wells (1302).

### Example 4: Antibody Discovery and Development

A method of antibody discovery and development is shown in FIG. 14. A subject (1403) is exposed to a pathogenic agent (1401). The subject is a human and the pathogenic agent is a virus as described herein. The target antigen is an anti-cancer PD-1 antigen. B cells (1405) are collected from the subject using known methods. The B cells are analyzed using single-cell arrays of nano-wells described herein (1406). Single cells of interest (1407) are isolated. The single cells secrete antibodies of interest (1408). The single cells are screened to identify specific parameters. This includes immunophenotyping (e.g. analysis of CD19, CD20, CD38, CD138), isotype Ig subtyping (e.g. IgG, IgM, IgA), affinity analysis, and antigen specificity. The parameters are related to secreted biomolecules of interest. The single cells are amplified (1409) and used to produce molecules of interest (1410) or the cells may be useful in a therapy. The cells of interest are used for immunotherapy. The recovered cells are antigen presenting cells involved in the CD40 pathway. Single-cell RT-PCR is performed on the individual recovered cells to recover paired antibody heavy and light (VH, VH) chain gene. Antibody optimization is performed. Recombinant paired antibody VH/VL chains are expressed to obtain recombinant therapeutic antibodies.

### Example 5: Live Single-Cell Metabolic Assay

In this example as seen in FIG. 15, a spectroscopic reference is acquired of amino acids in culture media (1505) present in sub-nanoliter wells (1501) by a laser scanning UV-Vis spectroscopic system (1504). The amino acid media (1505) is sealed in sub-nanoliter wells (1501) of a sub-nanoliter well array (1503) by a capture substrate (1502). The pathlength (1506) is defined as the depth of the nano-wells. Following acquisition of the reference spectrum, seed cells (1507) are loaded into the sub-nanoliter wells (1501) and the wells are re-sealed by the capture substrate (1502). Spectroscopic data is acquired of the live cells after every minute for a total of 100 minutes. Individual cells showing modified function (1508) are recovered. The effects of the amino acids on the cells is then correlated to cell function.

### Example 6: Prediction of Single-Cell Performances with High Accuracy

In this example as seen in FIG. 20, high preforming champion factories were selected with high efficiency. The vast majority of underperforming cells were excluded, by predicting the performance of the clones from the collected single-cell data with high accuracy. However, some underperforming cells based on the prediction were kept as controls for subsequent cultivation experiments.701

FIG. 20A shows a distribution of predicted single-cell performances. The high performing single-cell, highlighted in a circle, are very rare and hard to identify. FIG. 20B illustrates a tight correlation between single-cells performance and the scale-up performance of these clones on the bioreactor level. There was a strong (R>0.8) and significant (p<0.001) correlation of CSS with bioreactor titer. The circled clones of cells were proven to be champion factories with high bioreactor titers. This enables targeting high performance champion cells from a large cell pool with high precision, which was previously not possible, resulting from the platform described herein which is able to process more cells and perform analytics more accurately.

### Example 7: Clonal Stability

In this example as seen in FIG. 21, clonal manufacturing cell lines can undergo shifts over time due to issues in stability. This presents potential manufacturing issues, since the goal in process development and bio-manufacturing is to create processing conditions that consistently produce high yield and quality material. One potential issue is that it is difficult to accurately quantify expression changes on a cell-by-cell level in cell populations. As a solution, the disclosed platform provides a snapshot of a large population of cells on a cell-by-cell basis to identify phenotypic drift and predict potential manufacturing issues. This information can also be used for “real time” process optimization during process development activities. Accordingly, by monitoring shifts in a cell population on a cell-by-cell basis over time, process development and biomanufacturing can be improved.

### Example 8: Clonal Recovery

In this example as seen in FIG. 22, single cells were recovered, grew with high viability rates and an outgrowth rate of 30% to more than 70% (up to 95%), and an integrated monoclonality. Precise knowledge and control of cell location and recovery of “healthy” individual cells remains a key challenge, illustrated by the need to provide sufficient proof to regulatory agencies. In contrast to the current gold standard of preforming limiting dilutions or other emerging technologies, the technologies disclosed herein provides a high degree of monoclonality, as well as proof of monoclonality, by tracking all individual cells with high precision throughout the entire process. As exemplified in FIG. 22A, image confirmation for a single CHO cell before and after picking from a nanowell was provided. On Day 7 (FIG. 22B) and Day 14 (FIG. 22C), Clonal CHO cell culture in 96 well plate derived from the single-cell process disclosed herein. Furthermore, the process disclosed herein is extremely gentle on the cells, by creating a cell-friendly environment and avoiding harsh reagents and processing conditions (e.g., sheer force on the cells) which results in very high viability rates of the recovered cells (e.g., more than 90% viability after recovery).

### Example 9: Monoclonality Assurance

Assurance of clonality is crucial in cell line development both for the safety and efficacy, as well as quality and homogeneity of the product. Both FDA and the EMA request evidence of clonality, or otherwise can require additional manufacturing controls, which can delay and increase the cost of clinical trials, as well manufacturing. Gold standard technologies for cell line development such as limiting dilution or flow cytometry can only provide indirect proof of monoclonality, and therefore require additional rounds of single cell isolation. In contrast, the technology disclosed herein isolates single cells in nanowell arrays, and provides image-based proof of single cells at every step of the process, without ambiguity, and thus providing direct evidence of monoclonality.

To demonstrate monoclonality assurance, we obtained HEK293 cell lines that expressed GFP and RFP. Briefly, a total of 15,972 single RFP expressing cells and 1,019 single GFP expressing single cells were loaded on the array, resulting in a ratio of 6% GFP and 94% RFP cell population in the sample that was rich in RFP expressing cells. FIG. 23A shows the cell sample with GFP and RFP expressing cells was loaded onto a nanowell array chip. Imaging cytometry was performed, and fluorescence in green and red channels was recorded together with the brightfield images of the entire nanowell array. A software automatically identified the GFP and RFP expressing single cells in the nanowell array. A subset of GFP expressing cells among the presence of surrounding high population of RFP cells were then automatically recovered on the nanowell array. The recovered cells are expanded in the 96-well plate, and the plate was imaged at days 7 and 14 for fluorescence signal to demonstrate clonality without RFP signal (see FIG. 23B). As a control, randomly selected RFP expressing single cells were similarly recovered into the 96-well plate. Growth of the RFP expressing colony was observed as expected, and there was no signal present in the GFP channel at Day 14 (see FIG. 23C). Related statistics of the experiment are summarized in Table 1.

TABLE 1
Statistic of the Experiment on Monoclonality Assurance

| | Number or |
|---|---|
| Category | percentage |
| Total number of attempted GFP expressing cells | 81 |
| Total number of recovered GFP expressing cells | 81 |
| Number of wells with cells at Day 3 by visual inspection | 80 |
| under microscope | |
| Number of wells with GFP expressing cells with 2 or more | 52 |
| cells at Day 7 | |
| Number of wells with GFP expressing cells with 2 or more | 40 |
| cells at Day 14 (true positive) | |
| Number of wells with no GFP cells at Day 14 | 41 |
| Number of multicolor wells at Day 14 (false positive) | 0 |
| Overall cloning efficiency at Day 7 (true positive & growing | 64% |
| colonies/attempted seeded wells) | |
| Overall cloning efficiency at Day 14 (true positive & growing | 49% |
| colonies/attempted seeded wells) | |
| Probability of clonality (true positive/(true positive + false | 1 |
| positive)) | |
| One-sided upper 95% confidence interval by Wilson method | 94% |
| to take in account for sample size | |

### Example 10: Predictive Models for Clonal Selection Using Machine Learning

Cell morphology contains rich information about cellular state and variability, and image-based morphological profiling can yield extremely high numbers of morphological features. Furthermore, organelle content and productivity are highly correlated. Accordingly, capabilities of data analytics for improved predictivity for clone selection were expanded by processing additional cell morphology and product quality attribute parameters, and implementing a machine learning based approach.

As shown in FIG. 24, a machine-learning based approach for predicting clone performance was implemented. Training data set (cytometry images) were used for morphological cell feature (size, texture, nucleus size, organelles etc.) extraction by image analysis. The corresponding outcome data set (e.g. titer, growth, glycosylation, aggregation) was fed as output to an algorithm to train it with supervised machine learning. A second cell image data set was then used to score and evaluate the predictive power of the algorithm. The predictive power of the model algorithm for correct identification of high producing cell lines was optimized. For this, a classification model was used: multiclass logistic regression was first tested, multiclass boosted decision tree and neural network algorithms were used as needed. For prediction of numerical titer values, linear regression and neural network regression algorithms can be tested for optimizing the predictive power. Depending on the chosen approach, the final model could sort cells into discrete bins, such as low, mid, or high producers (classifier), or return numerical values for titer (regressor). The same methodology could be applied for other product quality attributes (e.g. high glycan or low glycan classification), cell growth and viability.

### Example 11: Cell Morphology Imaging

In this example, highest single cell throughput per acquisition time in class for morphological imaging is demonstrated. Parameter comparison between 384 well plate-based high content screening systems and the systems described herein is shown in Table 2.

For cell morphology imaging, higher magnification imaging is needed for resolving subcellular features. Increasing magnification levels may come at a cost of imaging time and throughput. In this regard, the technology disclosed herein is fastest in its class when compared with currently available systems/methods. For example, for the same 20× magnification, in comparison to 384-well plate high content screening systems, the systems and methods disclosed herein can image single cells in 80,000 to 1,000,000 wells within an order of magnitude shorter time. With larger number of camera pixels (e.g. for a 24 MP camera), the resolution can be improved 2 times over the current systems/methods down to diffraction limited resolution. (Table 2 shows the imaging time and resolution comparison for combination of 10× and 20× magnification objectives with some of the currently available camera options) Similarly, higher magnification objectives (e.g. 40×, 60×, etc.) can be used in the disclosed systems/methods to afford improved resolution. Even further improvements to the resolution beyond diffraction limit can be achieved with structured or multi-angle illumination and computational reconstruction. These are all afforded with the systems/methods disclosed herein to enable imaging with high speed, whereas the currently available high content screening systems are much slower in comparison and cannot afford higher magnification or additional described methods without sacrificing time or throughput beyond an acceptable level for cell viability.

TABLE 2
Parameters for cell morphology imaging

| | | | | Acquisition | | | | | | | |
|---|---|---|---|---|---|---|---|---|---|---|---|
| | Well | # Images | Total # | time (5 | | | | | Camera | pixel | |
| | diameter | collected | images | channels) | | | FOV | Camera | size | size | resolution |
| | (mm) | per well | for device | (hr) | Magnification | NA | (mm) | MP | (mm) | (um) | (um) |
| 384 well | 3.65 | 9 | 3456 | 3.5 | 20× (water | | 0.68 × 0.68 | 1 | | | 0.62 |
| plate - | | | | | immersion | | | | | | |
| based high | | | | | objective) | | | | | | |
| content | | | | | | | | | | | |
| screening | | | | | | | | | | | |
| systems | | | | | | | | | | | |
| high | 1.3 | 1 | 690 | 0.1 | 10× | 0.3 | 1.3 × 1.3 | 1 | 13.3 × 13.3 | 13 | 2.60 |
| throughput | 1.3 | 1 | 2760 | 0.4 | 20× | 0.7 | 0.65 × 0.65 | 1 | 13.3 × 13.3 | 13 | 1.30 |
| single-cell | 1.3 | 1 | 2380 | 0.35 | 20× | 0.7 | 0.7 × 0.7 | 5.5 | 16.6 × 14 | 6.5 | 0.65 |
| imaging | 1.3 | 1 | 690 | 0.1 | 10× | 0.5 | 1.3 × 1.3 | 24 | 13 × 15 | 2.75 | 0.55 |
| technology | 1.3 | 1 | 690 | 0.1 | 10× | 0.45 | 1.3 × 1.3 | 24 | 13 × 15 | 2.75 | 0.56 |
| | 1.3 | 1 | 2760 | 0.4 | 20× | 0.75 | 0.65 × 0.65 | 24 | 13 × 15 | 2.75 | 0.33 |

Note:
NA = numerical aperture:
FOV = Field of View

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for selecting a target cell; comprising:
a) placing a plurality of cells into a plurality of chambers, wherein each individual chamber of a subset of the plurality of chambers contains one or no more than 2, 3, 5, 10, 15 or 20 individual cells of the plurality of cells;
b) exposing at least the subset of the plurality of chambers from a) to a condition, wherein the condition is exposing the individual chamber with one or more regents, or treating the individual chamber with a plurality of secondary cells, or applying a membrane to the individual chamber to form an individual membrane-modified chamber, or contacting the individual chamber with a capture substrate, or contacting the individual chamber with a secondary cell-immobilized capture substrate, or a combination thereof;
c) detecting a signal or a change thereof from a particular chamber of the subset of the plurality of chambers during or after the exposing in b), wherein the signal or the change thereof is indicative of (i) the presence of a target cell in the particular chamber, or (ii) the presence of a product produced by the target cell in the particular chamber; and
d) selecting the target cell in the particular chamber from the plurality of cells at least based on a pre-determined value of the signal or the change thereof in c).

2. The method of claim 1, further comprising: e) transferring the target cell selected in d) to a cultivation vessel, and expanding the target cell into a colony or colonies in the cultivation vessel.

3. The method of claim 2, wherein the selecting in d) comprises predicting an expected outcome of the colony or colonies in e) based on the signal or the change thereof in c).

4. The method of any one of claims 1-3, wherein:
A) the plurality of cells in a) are from about 100 to about 1,000,000 heterogenous cells; and/or
B) a solution volume of the individual chamber is from 100 picoliter to 900 nanoliter; and/or
C) completing step a) is done in no more than 10, 9, 8, 7, 6, 5, 4, 3, 2, or 1 minute(s); and/or
D) completing steps a) to d) is done in no more than 48, 36, 24, 12, 10, 9, 8, 7, 6, 5, 4, 3, 2, or 1 hour, or 30, 20, 10, 5 minutes and/or
E) the detecting in c) is cell morphology imaging, near-infrared imaging, fluorescence imaging, luminescence imaging, UV-vis imaging, brightfield imaging, hyperspectral imaging, surface plasmon resonance (SPR) imaging, imaging with optical fibers, label-free imaging, mass spectrometry, or a combination thereof, and/or
F) the selecting in d) comprises analyzing (i) the signal or the change thereof, and/or (ii) an additional signal or a change thereof obtained from the colony or colonies in e), wherein the analyzing in F) is machine learning-based, or artificial intelligence (AI)-based, or deep learning-based, or neural networks-based, or a combination thereof; and/or
G) the expected outcome of an outgrowth population of the colony or colonies correlates with an observed outcome of the outgrowth population of the colony or colonies in e).

5. The method of claim 4, wherein the colony or colonies in e) displays higher monoclonality assurance when compared with a comparative colony or colonies obtained by (i) limiting dilution selection, (ii) fluorescence-activated cell sorting (FACS), (iii) isolating individual cells with cloning cylinders, or (iv) flow cytometry.

6. The method of claim 4, wherein the colony or colonies in e) displays higher viability when compared with a comparative colony or colonies obtained by (i) limiting dilution selection, (ii) fluorescence-activated cell sorting (FACS), (iii) isolating individual cells with cloning cylinders, or (iv) flow cytometry.

7. The method of claim 4, wherein the analyzing in F) comprises further analyzing intracellular staining for the product, and/or surface markers, and/or the cell morphology imaging against an optimized machine learning model built on correlating cell intracellular staining features, and/or surface markers, and/or cell morphological features of selected single cells with the corresponding product attribute parameters of the outgrowth populations derived from the selected single cells.

8. The method of claim 4, wherein the completing steps a) to d) in D) is from 48 to 36 hours, from 36 to 24 hours, from 24 to 12 hours, from 12 to 10 hours, from 10 to 9 hours, from 9 to 8 hours, from 8 to 7 hours, from 7 to 6 hours, from 6 to 5 hours, from 5 to 4 hours, from 4 to 3 hours, from 3 to 2 hours, from 2 to 1 hour(s), from 60 to 30 minutes, and from 30 to 1 minute(s).

9. The method of claim 4, wherein the completing steps a) to d) in D) is faster than when a comparative colony or colonies is obtained by (i) limiting dilution selection, or (ii) fluorescence-activated cell sorting (FACS), or (iii) isolating individual cells with cloning cylinders, or (iv) flow cytometry.

10. The method of claim 4, wherein completing steps b) to d) is done from 30 to 5 minutes, from 20 to 5 minutes, from 15 to 5 minutes, from 10 to 5 minutes.

11. The method of claim 4, wherein completing step d) is done from 10 to 9 minutes, from 9 to 8 minutes, from 8 to 7 minutes, from 7 to 6 minutes, from 6 to 5 minutes, from 5 to 4 minutes, from 4 to 3 minutes, from 3 to 2 minutes, from 2 to 1 minute(s), from 60 to 30 seconds, and from 30 to 1 second(s).

12. The method of claim 4, wherein steps b) and c) are performed while the plurality of cells receive reduced perturbations when compared with corresponding perturbations received by a comparative plurality of cells in a cell line development process of (i) limiting dilution selection, (ii) fluorescence-activated cell sorting (FACS), (iii) isolating individual cells with cloning cylinders, or (iv) flow cytometry.

13. The method of claim 12, wherein the perturbations are chemical, biological, or mechanical perturbations with regard to the plurality cells or the solution/environment of the plurality of cells.

14. The method of claim 4, wherein the target cell is not removed from the particular chamber before step d) is completed.

15. The method of claim 4, wherein the outcome comprise titer, cell growth metric, viable cell density, characteristics, expression of surface glycoproteins, glycosylation, phosphorylation, deamidation, methylation, acetylation aggregation, monoclonality, expression of cell markers, biological activities, or impurities.

16. The method of claim 15, wherein the analyzing in F) improves the correlation of the expected outcome of the outgrowth population of the colony or colonies with the observed outcome of an outgrowth population of the colony or colonies in e).

17. The method of any one of claims 1-16, wherein the product is an antibody, a monoclonal antibody, a biosimilar, a virus, a protein, a nucleotide, a bispecific, an antibody-drug conjugate, an exosome, a biomarker, or a metabolite.

18. A method for facilitating clone selection of a cell line, from among a plurality of candidate single cells, comprising:
a) generating, by an imaging unit, a first plurality of images of each of the plurality of candidate single cells individually, wherein each of the plurality of candidate single cells resides in an individual chamber of a plurality of chambers;
b) detecting, by one or more processors analyzing the first plurality of images for each of the plurality of candidate single cells, one or more cell features of each of the plurality of candidate single cells depicted in the first plurality of images; and
c) based on the one or more cell features, determining, by the one or more processors and according to a finalized single cell-to-colony machine learning model, one or more predicted attributes for a colony expanded from each of the plurality of candidate single cells;
d) ranking the plurality of candidate single cells according to the one or more predicted attributes for each of the plurality of candidate single cells,
wherein the finalized single cell-to-colony model predicts attributes of a hypothetical colony based on at least the one or more cell features of a single cell.

19. The method of claim 18, wherein the one or more cell features are morphological cell features of shape, size, color, pattern, texture, nucleus size, or organelles, or intracellular staining for a product produced by the single cell, or one or more surface markers, or a combination thereof.

20. The method of claim 18, wherein the one or more predicted attributes are titer, cell growth metric, viable cell density, characteristics, expression of surface glycoproteins, glycosylation, phosphorylation, deamidation, methylation, acetylation aggregation, monoclonality, expression of cell markers, biological activities, or impurities.

21. The method of any one of claims 18-20, wherein the finalized single cell-to-colony model is optimized by using a training data set comprising (i) the one or more morphological cell features from a second plurality of images for a plurality of training single cells, and (ii) measured quality attributes of each colony expanded from each of the plurality of training single cells.

22. The method of claim 21, wherein the finalized single cell-to-colony model is further optimized by (a) using a validation data set comprising (i) the one or more morphological cell features from a third plurality of images for a plurality of validation single cells, and (ii) measured quality attributes of each colony expanded from each of the plurality of validation single cells, and (b) comparing one or more predicted attributes of each of the plurality of validation single cells with the measured attributes of each of the colony expanded from each of the plurality of validation single cells.

23. A method for high-throughput cell line development, comprising:
a) providing a plurality of target cells, an array of nano-wells, one or more reagents and instructions;
b) loading the plurality of target cells into said array of nano-wells such that an individual well of the array of nano-wells contains an individual target cell;
c) exposing said plurality of target cells to one or more reagents;
d) obtaining measurements of individual target cells and quantitative measurements of individual articles associated with said individual target cells;
e) selecting a target cell from said individual target cells to be recovered based on predetermined values of said measurements;
wherein a time to reach a decision for selecting said target cell for recovery does not exceed 3 hours from the initialization of said method; and
wherein said method yields clones with a mean productivity of at least 5 grams per liter.

24. The method of claim 23, wherein a capture substrate is provided, further wherein one or more binding molecules for said article is immobilized to said capture substrate.

25. The method of claim 23 or 24, wherein said capture substrate is placed in proximity of said array of nano-wells before, during or after exposure of said one or more reagents to said target cells.

26. The method of claims 23, 24 or 25, wherein measurements of said articles are obtained on a surface of said capture substrate.

27. The method of any one of claims 23-26, wherein said measurements of said articles obtained on said surface of said capture substrate comprise optical analytics.

28. The method of any one of claims 23-27, wherein said article is a biomolecule.

29. The method of any one of claims 23-28, wherein said biomolecule is synthetically derived.

30. The method of any one of claims 23-29, wherein said biomolecule is naturally derived.

31. The method of any one of claims 23-30, wherein said biomolecule is a biomolecule comprising an Fc domain.

32. The method of any one of claims 23-31, wherein said biomolecule comprising an Fc domain is an antibody.

33. The method of any one of claims 23-32, wherein said article is secreted by said target cell.

34. The method of any one of claims 23-33, wherein said article is a bioparticle.

35. The method of any one of claims 23-34, wherein said article is presented on the surface of said target cell.

36. The method of any one of claims 23-35, wherein said article is internal to said target cell.

37. The method as in any one of claims 23-36, wherein said biomolecule is encoded by a heterologous gene.

38. The method as in any one of claims 23-37, wherein said target cell is a T cell, an antibody secreting cell, a B cell, a plasma cell, a hybridoma, an immune cell, or an engineered cell.

39. The method as in any of claims 23-38, wherein said engineered cell is a CHO cell, or HEK cell.

40. The method as in any one of claims 23-39, wherein said biomolecule binds to one or more antigens that are markers for infection.

41. The method as in any one of claims 23-40, wherein said infection is a viral infection, a parasitic infection, a bacterial infection, or a bioweapon-based infection.

42. The method as in any one of claims 23-41, wherein said viral infection is COVID-19.

43. The method as in any one of claims 23-42, wherein said infection is known to cause epidemic or pandemic levels of infection.

44. The method as in any one of claims 23-43, wherein said one or more reagents comprise one or more secondary cell, reporter cell, perturbing cell, one or more cellular factors, media, antigen, secondary binding molecule, labeling molecule, or a combination thereof.

45. The method as in any one of claims 23-44, wherein said one or more cellular factors are capable of modifying a cell in terms of parameters comprising growth, gene and protein expression, up-regulation, down-regulation, function, specificity, developmental timing, niche occupation, differentiation, de-differentiation, methylation, productivity, stability, glycosylation, aggregation, recombinant modification, genetic modification, transcriptional modification, modifications and interactions with proteins, methylation, ubiquitination, phosphorylation, or other perturbations.

46. The method as in any one of claims 23-45, wherein the number of target cells per array of nano-wells is less than or equal to 16,000.

47. The method as in any one of claims 23-46, wherein the number of target cells per array of nano-wells is less than or equal to 27,000.

48. The method as in any one of claims 23-47, wherein the number of target cells per array of nano-wells is less than or equal to 300,000.

49. The method as in any one of claims 23-48, wherein the number of target cells per array of nano-wells is less than or equal to 5,000.

50. The method as in any one of claims 23-49, wherein the volume of said target cells in a sample does not exceed 0.2 milliliters.

51. The method as in any one of claims 23-50, wherein the number of said target cells in a sample does not exceed 200,000 per milliliter.

52. The method as in any one of claims 23-51, wherein the number of said target cells in a sample does not exceed 20,000 per milliliter.

53. The method as in any one of claims 23-52, wherein the number of said target cells in a sample does not exceed 10,000 per milliliter.

54. The method as in any one of claims 23-53, wherein the number of said target cells in a sample does not exceed 2,000 per milliliter.

55. The method as in any one of claims 23-54, wherein the single-cell loading efficiency of cells is 33%.

56. The method as in any one of claims 23-55, wherein the single-cell loading efficiency of cells is 20%.

57. The method as in any one of claims 23-56, wherein the time for loading said individual target cells into said array of nano-wells and said secretion assay of said individual cells does not exceed 11 minutes.

58. The method as in any one of claims 23-57, wherein the time for loading said individual target cells into said array of nano-wells and secretion assay of said individual target cells does not exceed 6 minutes.

59. The method as in any one of claims 23-58, wherein the time for capturing biomolecules on said capture substrate after sealing said array of nano-wells does not exceed 29 minutes.

60. The method as in any one of claims 23-59, wherein the time for capturing biomolecules on the capture substrate surface after sealing said array of nano-wells does not exceed 11 minutes.

61. The method as in any one of claims 23-60, wherein the time for capturing biomolecules on said capture substrate after sealing said array of nano-wells does not exceed 4 minutes.

62. The method as in any one of claims 23-61, wherein said target cell does not contact detection reagents.

63. The method as in any one of claims 23-62, wherein the time to reach a decision for selecting said target cell does not exceed 2 hours from initialization of said method.

64. The method as in any one of claims 23-63, wherein the time to reach a decision for selecting said target cell does not exceed 4 hours from initialization of said method.

65. The method as in any one of claims 23-64, wherein the time to reach a decision for selecting said target cell does not exceed 5 hours from initialization of said method.

66. The method as in any one of claims 23-65, wherein the time to reach a decision for selecting said target cell does not exceed 1 hour from initialization of said method.

67. The method as in any one of claims 23-66, wherein the time to reach said decision for selecting said target cell does not exceed 5 doubling times.

68. The method as in any one of claims 23-67, wherein the time to reach said decision for selecting said target cell does not exceed 1 doubling time.

69. The method as in any one of claims 23-68, wherein said method yields clones with a mean productivity within a range of a 5 to 12 grams per liter.

70. The method as in any one of claims 23-69, wherein said method yields clones with a mean productivity within a range of 1 to 5 grams per liter.

71. The method as in any one of claims 23-70, wherein said method yields clones with a mean productivity within a range of 0.1 to 1 gram per liter.

72. The method as in any one of claims 23-71, wherein a collection of proof images is acquired at each step during the method.

73. The method as in any one of claims 23-72, wherein said capture substrate is comprised of a hard material.

74. The method as in any one of claims 23-73, wherein said capture substrate is comprised of a soft material.

75. The method as in any one of claims 23-74, wherein said array of nano-wells is comprised of a hard material.

76. The method as in any one of claims 23-75, wherein said array of nano-wells is comprised of a soft material.

77. The method as in any one of claims 23-76, wherein said hard material comprises a transparent plastic or a transparent glass material.

78. The method of as in any one of claims 23-77, wherein said substrate comprises a reflective material.

79. The method as in any one of claims 23-78, wherein said soft material comprises a transparent elastomeric material.

80. The method as in any one of claims 23-79, wherein said article is captured on said capture substrate.

81. The method as in any one of claims 23-80, wherein said article is captured on one or a plurality of beads inside of said well.

82. The method as in any one of claims 23-81, wherein said article is captured on an interior surface of said well.

83. The method as in any one of claims 23-82, wherein said article is captured within a matrix contained within said well.

84. The method as in any one of claims 23-83, wherein said measurements of individual target cells comprise characterizations of cellular objects, through segmentation or without segmentation, such as morphology, size, texture of nucleolus, endoplasmic reticulum, nucleoli, cytoplasmic RNA, actin, cytoskeleton, golgi, plasma membrane, mitochondria and other organelles or cell components or a combination thereof.

85. The method as in any one of claims 23-84, wherein data from said measurements of individual target cells is used to create a training data set to predict cellular function.

86. The method as in any one of claims 23-85, wherein said transgene is selected from the group consisting of amino acid (aa) pattern recognition receptor, killer activated receptor, killer inhibitor receptor, complement receptor, Fc receptor, major histocompatibility complex (MHC) molecule, human leukocyte antigen complex (HLA), cluster of differentiation (CD) markers, B cell receptor, T cell receptor, and a chimeric antigen receptor.

87. The method as in any of claims 23-86, wherein said direct measurements comprise bright field microscopy.

88. The method as in any one of claims 23-87, wherein said direct measurements comprise fluorescence microscopy.

89. A method for isolated co-culture utilizing a secondary cell suspension, comprising:
a) providing a plurality of target cells, an array of nano-wells, one or more reagents and instructions;
b) loading individual target cells of said plurality of target cells into said array of nano-wells;
c) applying a membrane to said array of nano-wells to form a membrane-modified array of nano-wells;
d) providing a suspension of a plurality of secondary cells, one or more reagents, or a combination thereof, near or in contact with said membrane-modified array of nano-wells;
e) obtaining measurements of individual target cells and measurements of individual articles associated with said individual target cells;
f) selecting a target cell from said individual target cells to be recovered based on predetermined values of said measurements; and
wherein a time to reach a decision for selecting said target cell for recovery does not exceed 3 hours from the initialization of said method.

90. The method as in claim 89, wherein said plurality of secondary cells reside in a chamber that is fluidically connected to a flow cell containing the membrane-modified array of nano-wells.

91. The method as in claim 89 or 90, wherein the flow rate of said secondary cell suspension, said one or more reagents, or a combination thereof is equal to or greater than about 0 milliliters per minute.

92. A method for isolated co-culture utilizing a secondary cell immobilized-capture substrate, comprising:
a) providing a plurality of target cells, an array of nano-wells, one or more reagents, instructions; and a plurality of secondary cells immobilized to a capture substrate;
b) loading individual target cells of said plurality of target cells into said array of nano-wells;
c) applying a membrane to said array of nano-wells to form a membrane-modified array of nano-wells;
d) simultaneously contacting said membrane-modified array of nano-wells and the secondary cell-immobilized capture substrate with one or more reagents;
e) obtaining measurements of individual target cells and measurements of individual articles associated with said individual target cells;
f) selecting a target cell from said individual target cells to be recovered based on predetermined values of said measurements;
wherein a time to reach a decision for selecting said target cell for recovery does not exceed 3 hours from the initialization of said method; and
wherein said method yields clones with a mean productivity of 5 grams per liter.

93. A system for high-throughput cell line development, comprising:
a) an array of nano-wells comprising individual nano-wells, wherein said individual nano-wells contain zero or more target cells;
b) an apparatus for reversibly sealing a capture substrate with said array of nano-wells;
c) a reagent module configured for supplying one or more reagents to said array of nano-wells;
d) a detection module configured for performing measurements of biomolecules secreted by said target cell onto said capture substrate at discrete positions indexed to said individual wells;
e) a cell recovery apparatus configured for recovery of said individual cells, wherein values extracted from said measurements of biomolecules and cells are compared to predetermined criteria and used for the selection of said individual cells to be recovered;
wherein said system is configured to reach a decision for selecting said target cell for recovery within 3 hours from initialization; and
wherein said system is configured to yield clones with a mean productivity of 5 grams per liter.

94. The system of claim 93, wherein the system comprises an apparatus configured for sealing a capture substrate to said array of nano-wells, whereupon sealing a substantially aligned and substantially fluid tight seal between the one or more capture substrates and the one or more array of nano-wells is made.

95. The system as in any one of claims 93-94, wherein said direct measurements comprise bright field microscopy measurements.

96. The system as in any one of claims 92-95, wherein said direct measurements comprise microscopy measurements utilizing a laser source and a photomultiplier tube for detection.

97. The system as in any one of claims 92-96, wherein the system comprises a controller configured for actuating said system and analyzing data.

98. The system as in any one of claims 92-97, wherein a well of said array of nano-wells has a diameter of 5 to 150 microns.

99. The system of claim 98, wherein said well has a volume of picoliters to 15 nanoliters.

100. The system of claim 98, wherein said well has a volume of 250 picoliters.

101. The system as in any one of claims 98-100, wherein said well comprises shapes of circle, oval, square, triangle, diamond, or rectangle or combination thereof.

102. The system as in any one of claims 98-101, wherein said well has a depth of 25 microns.

103. The system as in any one of claims 98-102, wherein said well has a depth of 100 microns.

104. The system as in any one of claims 98-103, wherein said well has a depth of 250 microns.

105. The system as in any one of claims 98-104, wherein said well has a diameter to depth ratio of 1/10 to 4.

106. The system as in any one of claims 92-105, wherein the number of wells per array is about 1 million to about 10 million.

107. The system as in any one of claims 92-106, wherein the number of wells per array is about 100,000 to about 1 million.

108. The system as in any one of claims 92-107, wherein the number of wells per array is about 10,000 to about 100,000.

109. The system as in any one of claims 92-108, wherein the number of cells per a well of said array of nano-wells from zero to about 10.

110. The system as in any one of claims 92-109, wherein a plate comprises a plurality of said array of nano-wells.

111. The system as in any one of claims 92-110, wherein said plate comprises a plurality of recesses.

112. The system as in any one of claims 92-111, wherein a recess of said plurality of recesses comprises an array of nano-wells.

113. The system as in any one of claims 92-112, wherein said capture substrate comprises a sensing surface.

114. The system as in any one of claims 92-113, wherein said array of nano-wells comprises said sensing surface.

115. The system as in any one of claims 92-114, wherein said sensing surface comprises a layered semiconductor.

116. The system as in any one of claims 92-115, wherein said sensing surface is configured for reflection mode imaging for real-time endpoint detection of binding on said sensing surface.

117. The system as in any one of claims 92-116, wherein said sensing surface is configured for surface plasmon resonance detection of said articles.

118. The system as in any one of claims 92-117, wherein said sensing surface is configured for interferometric detection of said articles.

119. The system as in any one of claims 92-118, wherein said sensing surface is configured for whispering gallery mode detection of said articles.

120. A mechanism sealing a capture substrate to an array of nano-wells in a sterile fashion, comprising:
a) a top piece configured to immobilize capture substrate;
b) a base configured to immobilize an array of nano-wells;
wherein said base comprises one or more alignment rods to align said top piece to said base such that said capture substrate and said array of nano-wells are fixed in coplanar and rotationally aligned orientation; and
wherein the distance between said capture substrate and said array of nano-wells can be controllably varied along an axis perpendicular to the coplanar planes of said capture substrate and said array of nano-wells, thus placing said capture substrate and said array of nano-wells in alignment and forming a sterile, fluid tight seal.

121. The mechanism as in claim 120, wherein said distance is minimized to form a seal between said capture substrate and said array of nano-wells that is substantially aligned and substantially fluid tight.

122. The mechanism as in claim 120 or 121, wherein said capture substrate is aligned with said array of nano-well in a coplanar orientation and in proximity simultaneously with a plurality of capture substrates and a plurality of array of nano-wells.

123. The mechanism as in claim 120, 121 or 122, wherein one or more of said array of nano-wells are contained within a plate.

124. The mechanism as in any one of claims 120-123, wherein said plate comprises one or more recesses, wherein each recess contains one or more of said arrays of nano-wells.

125. The mechanism as in any one of claims 120-124, wherein said plate comprises one or more recesses, wherein an array of nano-wells can be placed and removed from a recess of said one or more recesses.

126. The mechanism as in any one of claims 120-125, wherein a specific force is applied equally across a region of said capture substrate, said array of nano-wells, or a combination of both wherein a predetermined pressure applied across said region is substantially uniform.

127. The mechanism as in any one of claims 120-126, wherein said recess comprises one or more channels configured to accept fluid displaced between said capture substrate and said array of nano-wells.

128. The mechanism as in any one of claims 120-127, wherein said recess comprises one or more ridges to contain and align said capture substrate relative to said array of nano-wells.

129. The mechanism as in any one of claims 120-128, wherein said recess further comprises an alignment recess configured to align said capture substrate relative to said array of nano-wells.

130. The mechanism as in any one of claims 120-129, wherein said recess contains channels configured to form a pedestal and wherein said capture substrate contains a capture substrate-recess configured to accept the pedestal, allowing for alignment between said capture substrate and said pedestal.

131. The mechanism as in any one of claims 120-130, wherein said plate is in fluidic connection with one or more reservoirs wherein said one or more reservoirs contain said one or more reagents.

132. A method for cell line development utilizing a terminal assay for cell selection, comprising:
a) providing a plurality of target cells, an array of nano-wells, one or more reagents and instructions;
b) loading individual target cells of said plurality of target cells into individual nano-wells of said array of nano-wells and exposing said individual target cells to one or more reagents;
c) contacting a capture substrate to said array of nano-wells, thereby sealing said individual target cells into said individual nano-wells;
d) growing a colony of cells from said individual target cell, such that one or more colony cells of the colony of cells are transferred to said capture substrate onto positions registered to corresponding individual wells from which said one or more colony cells originated;
e) separating said capture substrate from said array of nano-wells and performing an assay that may result in the death of said colony cells that transferred to said indexed positions; and
f) selecting colony cells for recovery from said corresponding individual wells based on predetermined values of said measurements.

133. The method as in claim 132, wherein a capture substrate is sealed onto said array of nano-wells, wherein each well is sealed by said capture substrate, wherein some cells of said single-well colony are attached to said capture substrate at locations on said capture substrate in which said locations are registered to said well position in said array of nano-wells.

134. The method as in any one of claims 132 or 133, wherein said capture substrate is separated from said array of nano-wells and measurements are performed on said some cells that are attached to said capture substrate at said locations.

135. The method as in any one of claims 132, 133, or 134, wherein said measurements are performed on said individual cells in said individual wells, prior to colony growth.

136. The method as in any one of claims 132-135, wherein said measurements are performed on said single-well colony of target cells.

137. The method as in any one of claims 132-136, wherein said measurements comprise image cytometry or a secretion assay.

138. The method as in any one of claims 132-137, wherein said measurements are used to determine identity of said individual cells.

139. The method as in any one of claims 132-138, wherein living cells within said single-well colony of target cells or clones are recovered based from said array of nano-wells or said capture substrate or a combination thereof.

140. A method for high-throughput identification of a B cell or antibody secreting cells (ASCs), comprising:
(a) obtaining a plurality of B cells or ASCs from a subject;
(b) loading the individual B cells or ASCs into individual wells of an array of nano-wells;
(c) detecting a secreted product of the individual B-cell or ASCs;
(d) selecting the individual B-cell or ASCs;
wherein a time to reach a decision for selecting said target cell does not exceed 5.5 hours from the initialization of said method.

141. The method of claim 140, wherein the subject has been immunized naturally through infection with pathogenic agent.

142. The method of claim 140 or claim 141, wherein the pathogenic agent is a virus selected from the group consisting of: SARS-CoV-2, Herpes simplex virus (HSV), varicella zoster virus, cytomegalovirus (CMV), Epstein-Barr virus (EBV), Eastern equine encephalitis (EEE), western equine encephalitis (WEE), rubella virus, poliovirus, coxsackievirus, an enterovirus, St. Louis encephalitis (SLE), Japanese encephalitis, rubeola (measles) virus, mumps virus, California encephalitis, LaCrosse virus, human immunodeficiency virus (HIV), rabies virus, WNV, dengue, AAV and Influenza A virus.

143. The method as in any one of claims 140-142, wherein the subject has been immunized with a target antigen.

144. The method as in any one of claims 140-143, wherein the subject comprises a human.

145. The method as in any one of claims 140-144, wherein the subject comprises a non-human.

146. The method as in any one of claims 140-145, wherein the antigen comprises a viral antigen, self-antigen or tumor antigen.

147. The method as in any one of claims 140-146, wherein the property comprise an article produced by an individual B-cell or ASC.

148. The method as in any one of claims 140-147, wherein the article comprises an antibody.

149. The method as in any one of claims 140-148, wherein the article comprises a secreted molecule.

150. The method as in any one of claims 140-149, wherein the secreted molecule comprises a cytokine.

151. The method as in any one of claims 140-150, wherein the property comprises an interaction between the individual B cell and a second cell or second biomolecule.

152. A method cell line development utilizing identification of glycosylation patterns on a biomolecule, comprising:
a) providing a plurality of target cells, an array of nano-wells, one or more reagents and instructions;
b) loading the plurality of target cells into an array of nano-wells such that an individual well of the array of nano-wells contains an individual target cell;
c) exposing the plurality of target cells to one or more reagents, wherein each individual target cell can produce a biomolecule;
d) capturing said biomolecule produced by each individual target cell on a capture substrate; wherein said capture substrate is configured to keep the biomolecule produced by each individual target cell in the array of nano-wells spatially distinct and registered to said individual nano-well of origin; wherein the capture substrate comprises a glycan binding reagent;
e) comparing said captured biomolecule produced by each individual target cell in the array of nano-wells on the capture substrate to a reference;
f) ascertaining a glycosylation profile for each individual biomolecule; and
g) selecting a target cell from the plurality of target cells to be recovered based on the glycosylation profile of the biomolecule produced by the individual target cell.

153. The method of claim 152, wherein a time to reach a decision for selecting the individual target cell for recovery does not exceed 3 hours from the initialization of the method.

154. The method of claim 152 or claim 153, wherein the method yields clones with differentiated glycan profiles.

155. The method of any one of claims 152, 153 or 154, wherein capture substrates are used to create sequential prints.

156. The method of any one of claims 152-155, wherein the glycan binding reagent is a lectin, an antibody, or an antibody mimetic.

157. The method of any one of claims 152-156, wherein one or more receptors for the biomolecule is immobilized to said capture substrate.

158. The method of any one of claims 152-157, wherein the capture substrate is placed in proximity of the array of nano-wells before, during or after exposure of one or more reagents to said target cells.

159. The method of any one of claims 152-158, wherein measurements of the biomolecules are obtained on a surface of said capture substrate.

160. The method of any one of claims 152-159, wherein the measurements of said biomolecules obtained on said surface of said capture substrate comprise bright field microscopy, fluorescence microscopy, microscopy utilizing a laser source and a photomultiplier tube detector, or a combination thereof.

161. The method of any one of claims 152-160, wherein the biomolecule is a secreted bio-molecule.

162. The method of any one of claims 152-161, wherein the biomolecule comprising an Fc domain.

163. The method of any one of claims 152-162, wherein the biomolecule comprising an Fc domain is an antibody.

164. The method of any one of claims 152-163, wherein said biomolecule is secreted by said target cell.

165. The method of any one of claims 152-164, wherein said biomolecule is a bioparticle.

166. The method of any one of claims 152-165, wherein said biomolecule is presented on the surface of said target cell.

167. The method of any one of claims 152-166, wherein the reference comprises a reference glycan profile.

168. The method of any one of claims 152-167, wherein the reference is a glycan profile reference.

169. The method of any one of claims 152-168, wherein said biomolecule is internal to said target cell.

170. A method for cell line development utilizing mass spectrometry for identification of a biomolecule, comprising:
a) providing a plurality of target cells, an array of nano-wells, one or more reagents and instructions;
b) loading the plurality of target cells into array of nano-wells such that an individual well of the array of nano-wells contains an individual target cell;
c) exposing the plurality of target cells to one or more reagents, wherein each individual target cell can produce a biomolecule;
d) capturing each biomolecule produced by each individual target cell on a capture substrate; wherein the capture substrate is configured to keep each biomolecule produced by each individual target cell in the array of nano-wells spatially distinct and registered to said individual well from which said individual target cell originated;
e) analyzing the biomolecule with mass spectrometry (MS);
f) comparing and the captured biomolecule on the capture substrate to a mass spectrometry-based reference;
g) identifying the captured biomolecule; and
h) selecting a target cell from the individual target cells to be recovered based on the identify of biomolecule produced by the individual target cell.

171. The method of claim 170, wherein a time to reach a decision for selecting the individual target cell for recovery does not exceed 3 hours from the initialization of the method.

172. The method as in any one of claims 170-171, wherein the method yields clones with differentiated Glycan profile.

173. The method as in any one of claims 170-172, wherein the capture substrate is used to create sequential prints.

174. The method as in any one of claims 170-173, wherein the capture substrate comprises a MS compatible capture slide.

175. The method as in any one of claims 170-174, wherein said mass-spectrometry is MALDI-TOF mass spectrometry.

176. The method as in any one of claims 170-175, wherein said mass-spectrometry is MALDI-MSI.

177. The method as in any one of claims 170-176, wherein the reference is a reference MS profile.

178. The method as in any one of claims 170-177, wherein array of nano-wells comprises individual nano-wells 50 microns in diameter, 100 microns deep.

179. The method as in any one of claims 170-178, wherein said individual nano-wells are packed in arrangements comprising hexagonal and square.

180. The method as in any one of claims 170-179, the center to center spacing for nano-wells in the array of nano-wells is 100 microns.

181. A method of selecting a target cell based on an aggregation property of a secreted biomolecule, comprising:
a) providing a plurality of cells, an array of nano-wells, and one or more reagents, wherein the plurality of cells comprises a target cell;
b) loading the plurality of cells into said array of nano-wells such that an individual well of the array of nano-wells contains an individual cell;
c) exposing the plurality of cells to one or more reagents, wherein each of the plurality of cells can produce a biomolecule; wherein the target cell can produce a target biomolecule;
d) capturing said biomolecule produced by each individual target cell on a capture substrate; wherein said capture substrate is configured to keep said biomolecule produced by each individual cell in the array of nano-wells spatially distinct and registered to said individual nano-well from which said biomolecule originated;
e) exposing the biomolecule or cell to a reagent or other perturbant that induces aggregation of the biomolecule; determining an aggregation property of the biomolecule to identify the target biomolecule; and
f) selecting the target cell from the plurality of cells to be recovered based on the aggregation property of the target biomolecule.

182. The method as in any one of claim 181, wherein a time to reach a decision for selecting the individual target cell for recovery does not exceed 3 hours from the initialization of the method.

183. The method as in claim 181 or claim 182, wherein the method yields clones with less than 7 percent aggregation.

184. The method as in any one of claims 181-183, wherein said biomolecule comprises an Fc domain.

185. The method as in any one of claims 181-184, wherein said biomolecule comprising an Fc domain is an antibody.

186. The method as in any one of claims 181-185, wherein the biomolecule is secreted by the target cell.
